(19) United States
(12) Patent Application Publication
(10) Pub. No.: US 2024/0066072 A1
(43) Pub. Date: Feb. 29, 2024

(54) CLOSTRODIOIDES DIFFICILE TREATMENT

(71) Applicant: The Regents of the University of Michigan, Ann Arbor, MI (US)

(72) Inventors: Nobuhiko Kamada, Ann Arbor, MI (US); Hiroko Kitamoto, Ann Arbor, MI (US)

(21) Appl. No.: 17/764,878

(22) PCT Filed: Sep. 28, 2020
(86) PCT No.: PCT/US2020/053023

§ 371 Date: Mar. 29, 2022

Related U.S. Application Data
(60) Provisional application No. 62/909,954, filed on Oct. 3, 2019.

(51) Int. Cl. A61K 35/74; A61K 38/20; A61P 31/04; G01N 33/68

(52) U.S. Cl. CPC A61K 35/74; A61K 38/20; A61P 31/04; G01N 33/6869; A61K 2035/115

(57) ABSTRACT

Provided herein is technology relating to preventing and treating gastrointestinal dysbiosis and particularly, but not exclusively, to compositions, methods, systems, and kits for treating and/or preventing Clostridioides difficile infection in an organism.

### STATEMENT REGARDING FEDERALLY SPONSORED RESEARCH OR DEVELOPMENT

This invention was made with government support under DK108901 awarded by the National Institutes of Health. The government has certain rights in the invention.

This application is a § 371 National Phase entry of International Application No. PCT/US2020/053023, filed on Sep. 28, 2020, which claims priority to U.S. Provisional Patent Application No. 62/909,954, filed Oct. 3, 2019, which are incorporated herein by reference in their entireties.

### SEQUENCE LISTING

The computer readable sequence listing filed herewith, titled “UM-38044-252_SQL”, created Sep. 13, 2022, having a file size of 31,182 bytes, is hereby incorporated by reference in its entirety.

### FIELD

Provided herein is technology relating to preventing and treating gastrointestinal dysbiosis and particularly, but not exclusively, to compositions, methods, systems, and kits for treating and/or preventing Clostridioides difficile infection in an organism.

### BACKGROUND

Clostrodioides difficile is a major cause of nosocomial infections in hospitalized patients (1, 2). Patients treated with antibiotics, who have had gastrointestinal surgery, and/or who are immunocompromised have a higher risk of infection. Clostrodioides difficile infection (CDI) causes symptoms such as watery diarrhea, fever, loss of appetite, nausea, and abdominal pain and/or tenderness and can cause pseudomembranous colitis (PMC), toxic megacolon, perforations of the colon, sepsis, and death. CDI occurs worldwide and increase health care costs in the United States by at least $1.5 billion per year (see, e.g., Lessa (2012) “Current status of Clostridium difficile infection epidemiology” Clinical Infectious Diseases 55 Suppl 2: S65 - 70; Leffler (2015) “Clostridium difficile infection” The New England Journal of Medicine 372: 1539 - 48, each of which is incorporated herein by reference. Accordingly, treatments to prevent and/or ameliorate CDI are needed.

### SUMMARY

C. difficile does not grow and cause disease in healthy individuals having intact resident microbiota. In contrast, disruption of the normal microbiota (e.g., by antibiotics) promotes the growth of C. difficile and can cause CDI (3, 4). Restoration of the normal microbial structure (e.g., by treatment with a healthy microbiota (e.g., by fecal microbiota transplantation (FMT))) cures greater than 95% of recurrent CDI (1). Thus, the gut microbiota plays a central role in the prevention of CDI (1, 2, 4).

Prior to the experiments conducted during the development of embodiments of the technology described herein, mechanisms by which the constituents of a healthy gut microbiota prevent colonization and growth of C. difficile were not fully understood. Previous studies have indicated that the metabolic functions of the gut microbiota increase the abundance of metabolites that interfere with the growth of C. difficile and decrease the availability of luminal metabolites that promote the germination and/or growth of C. difficile (7, 8, 9, 10). For example, the gut microbiota converts primary bile acids to secondary bile acids, generates short-chain fatty acids (SCFAs) from dietary fibers, and consumes various luminal nutrients (5, 6). These functions of the gut microbiota contribute to preventing C. difficile growth in the gut. In addition, gut microbiotas activate host antimicrobial immunity (e.g., development of T helper 17 (Th17) cells, production of IgA, production of antimicrobial peptides, etc.), which prevents the colonization and/or growth of many enteric pathogens (3, 5). However, prior to experiments conducted during the development of embodiments of the technology provided herein, the role of host immunity-mediated colonization resistance in CDI, conferred by the gut microbiota, was underappreciated.

Accordingly, in some embodiments, the technology provided herein relates to a method of treating a subject for a Clostridioides difficile infection. For example, in some embodiments, methods comprise administering a composition comprising Phascolarctobacterium sp. to a subject in need of treatment for a Clostridioides difficile infection; and increasing the level of interleukin-22 (IL-22) in the gut of said subject. In some embodiments, the subject has gut dysbiosis and/or low levels of Phascolarctobacterium spp. In some embodiments, the subject has a Clostridioides difficile infection (CDI). In some embodiments, the subject has an increased risk of having a CDI. In some embodiments, the subject has one or more of diarrhea, ulcerative colitis, colitis, Crohn's disease, irritable bowel disease, and/or inflammatory bowel disease. The technology is not limited by the method of increasing IL-22 in the gut. For example, in some embodiments, increasing the level of IL-22 in the gut of the subject comprises administering IL-22 to the subject. In some embodiments, increasing the level of IL-22 in the gut of the subject comprises administering a microorganism to the subject that induces production of IL-22 in the gut. In some embodiments, increasing the level of IL - 22 in the gut of the subject comprises administering a recombinant microorganism that produces IL - 22 to the subject. In some embodiments, administering a composition comprising Phascolarctobacterium sp. to a subject in need of treatment for a Clostridioides difficile infection and increasing the level of interleukin-22 (IL-22) in the gut of the subject are performed simultaneously or substantially or essentially simultaneously. In some embodiments, administering a composition comprising Phascolarctobacterium sp. to a subject in need of treatment for a Clostridioides difficile infection and increasing the level of interleukin-22 (IL-22) in the gut of the subject are performed sequentially, though the technology does not limit the method to which step is performed first and/or for how many times a step is repeated, if at all. The technology is not limited in the Phascolarctobacterium sp. that is administered. For example, in some embodiments, the Phascolarctobacterium sp. is P. faecium and/or P. succinatutens.

In some embodiments, a subject is tested for the presence, absence, and/or amount of Phascolarctobacterium sp. in the gut. Accordingly, in some embodiments, methods further comprise testing a subject for the level of Phascolarctobacterium sp. in the gut. In some embodiments, a subject is tested for the presence, absence, and/or amount of Clostridioides difficile in the gut. Accordingly, in some embodiments, methods further comprise testing said subject for the level of Clostridioides difficile in the gut. In some embodiments, a subject is tested for the presence, absence, and/or amount of IL-22 in the gut. Accordingly, in some embodiments, methods further comprise testing the subject for the level of IL-22 in the gut. In some embodiments, a subject is tested for the presence, absence, and/or amount of glycans in the gut. Accordingly, in some embodiments, methods further comprise testing the subject for the level of glycans in the gut. In some embodiments, a subject is tested for the presence, absence, and/or amount of succinate in the gut. Accordingly, in some embodiments, methods further comprise testing the subject for the level of succinate in the gut. In some embodiments, a subject is tested for the presence, absence, and/or amount of MGAT4A expression or MGAT4A protein in the gut. Accordingly, in some embodiments, methods further comprise testing the subject for the level of MGAT4B expression or MGAT4B protein in the gut. In some embodiments, a subject is tested for the presence, absence, and/or amount of MGAT4B expression or MGAT4B protein in the gut. Accordingly, in some embodiments, methods further comprise testing the subject for the level of MGAT4A expression or MGAT4A protein in the gut. In some embodiments, methods further comprise obtaining or providing a sample from said subject.

In some embodiments, the technology relates to a composition comprising Phascolarctobacterium sp. and one or more of IL-22; a recombinant organism expressing IL-22; and/or an organism that increases IL-22 in the gut. In some embodiments, the composition is formulated for administration to a subject in need of a treatment for CDI. In some embodiments, the composition is formulated for enteric administration, e.g., oral or rectal administration. In some embodiments, the composition comprises no more than one species, strain, and/or operational taxonomic unit of bacteria. In some embodiments, the composition comprises no more than two species, strains, and/or operational taxonomic units of bacteria. In some embodiments, the composition comprises no more than three species, strains, and/or operational taxonomic units of bacteria. In some embodiments, the composition comprises no more than four species, strains, and/or operational taxonomic units of bacteria. In some embodiments, the composition comprises no more than five species, strains, and/or operational taxonomic units of bacteria. In some embodiments, the composition comprises microorganisms consisting of one or more of Phascolarctobacterium sp., a recombinant organism expressing IL-22; and/or an organism that increases IL-22 in the gut. In some embodiments, the compositions further comprise a prebiotic. In some embodiments, compositions comprise 10⁴, 10⁵, 10⁶, 10⁷, 10⁸, or 10⁹ CFU of Phascolarctobacterium sp. In some embodiments, the composition is formulated for administration to a human less than 1 year old. In some embodiments, the composition is formulated for administration to a human 1-10 years old. In some embodiments, the composition is formulated for administration to a human 10-30 years old. In some embodiments, the composition is formulated for administration to a human 30-60 years old. In some embodiments, the composition is formulated for administration to a human more than 60 years old.

Some embodiments provide a kit comprising a composition comprising Phascolarctobacterium sp. In some embodiments, kits further comprise one or more of IL-22; a recombinant organism expressing IL-22; and/or an organism that increases IL-22 in the gut. In some embodiments, kits further comprise a diluent and/or a component for administering said composition to a subject. In some embodiments, kits comprise multiple doses of one or more of said compositions. In some embodiments, kits comprise components for diagnosing a subject. For example, in some embodiments, kits comprise oligonucleotides complementary to a nucleic acid from Phascolarctobacterium sp. or Clostridioides difficile. In some embodiments, kits comprise an antibody specific for a protein of Phascolarctobacterium sp. or Clostridioides difficile. In some embodiments, kits comprise a component for obtaining a sample from a subject.

Some embodiments provide a system comprising a composition comprising Phascolarctobacterium sp. In some embodiments, systems further comprise one or more of IL-22; a recombinant organism expressing IL-22; and/or an organism that increases IL-22 in the gut. In some embodiments, systems further comprise a diluent and/or a component for administering said composition to a subject. In some embodiments, systems further comprise oligonucleotides complementary to a nucleic acid from Phascolarctobacterium sp. or Clostridioides difficile. In some embodiments, systems further comprise an antibody specific for a protein of Phascolarctobacterium sp. or Clostridioides difficile. In some embodiments, systems further comprise a component for obtaining a sample from a subject. In some embodiments, systems further comprise a thermocycler and polymerase. In some embodiments, systems further comprise a culture medium for growing Phascolarctobacterium sp. or Clostridioides difficile.

In some embodiments, the technology provides a method for identifying a subject in need of a treatment for Clostridioides difficile. For example, in some embodiments, the methods comprise detecting a decreased amount of Phascolarctobacterium spp. in a sample from a subject; and/or detecting a decreased amount of IL-22 in a sample from a subject, wherein detecting a decreased amount of Phascolarctobacterium spp. in a sample from a subject and/or detecting a decreased amount of IL-22 in a sample from a subject identifies the subject from whom the sample was taken as a subject in need of a treatment for Clostridioides difficile. In some embodiments, methods for identifying a subject in need of a treatment for Clostridioides difficile comprise detecting a decreased amount of glycan in a sample from said subject and/or an increase of succinate in a sample from the subject. In some embodiments, a sample from the subject is a fecal sample or is obtained from the gut of said subject.

In related embodiments, the technology provides use of a composition comprising Phascolarctobacterium sp. to treat a subject for a Clostridioides difficile infection. In some embodiments, the composition further comprises one or more of IL-22; a recombinant organism expressing IL-22; and/or an organism that increases IL-22 in the gut. In some embodiments, the subject has one or more of diarrhea, ulcerative colitis, colitis, Crohn's disease, irritable bowel disease, and/or inflammatory bowel disease. In some embodiments, the composition is formulated for enteric administration. In some embodiments, the composition comprises no more than one species, strain, and/or operational taxonomic unit of bacteria. In some embodiments, the composition comprises no more than two species, strains, and/or operational taxonomic units of bacteria. In some embodiments, the composition comprises no more than three species, strains, and/or operational taxonomic units of bacteria. In some embodiments, the composition comprises no more than four species, strains, and/or operational taxonomic units of bacteria. In some embodiments, the composition comprises no more than five species, strains, and/or operational taxonomic units of bacteria. In some embodiments, the composition comprises 10⁴, 10⁵, 10⁶, 10⁷, 10⁸, or 10⁹ CFU of said Phascolarctobacterium sp.

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

These and other features, aspects, and advantages of the present technology will become better understood with regard to the following drawings:

FIGS. 1A-1C show that healthy human microbiotas prevent C. difficile infection. FIG. 1A (left) is a plot showing C. difficile load in feces as a function of time after infection. GF B6 mice were colonized with healthy control (HC) microbiotas for 2 weeks (human microbiota-associated (HMA) mice). GF or HC-HMA mice were then infected with C. difficile VPI 10463 spores (10 3 spores/mouse). C. difficile load in feces was determined on indicated days post infection. Dots represent individual mice. Data are presented as median values (N=3-9). FIG. 1A (right) is a plot showing the mortality of C. difficile infected GF or HC-HMA mice. ****; P<0.0001 by Log-rank test.

FIG. 1B (left) is a plot showing CFU of C. difficile in feces and FIG. 1B (right) is a plot of mortality for HC-HMA mice treated with a cocktail of antibiotics or regular water (N=5) and then infected with C. difficile VPI spores. CFU in feces (left. **; P<0.01 by Mann-Whitney U test) and mortality (right. ****; P<0.0001 by Log-rank test.)

FIG. 1C shows representative histological images and associated histological scores. Scale bar is 100 μm. Data are presented as mean values. **; P<0.0001 by Mann-Whitney U test.

FIGS. 2A-2D show that healthy human microbiota-driven IL-22 prevents C. difficile blooms in the gut. FIG. 2A (left) is a plot showing C. difficile load in feces as a function of time post infection for GF-Rag1^{-/-} mice colonized with healthy microbiotas for 2 weeks (HC-HMA-Rag1^{-/-} mice). GF or HC-HMA-Rag1^{-/-} mice were infected with C. difficile VPI 10463 spores (10³ spores/mouse). C. difficile load in feces was determined on indicated days post infection. Dots represent individual mice. FIG. 2A (right) is a plot showing the mortality of C. difficile infected mice. **; P<0.01 by Log-rank test.

FIG. 2B is a bar plot of Il22 mRNA levels in GF-Rag1^{-/-} or HC-HMA-Rag1−/− mice (N=3-6) measured by qPCR. Expression was normalized to that of the murine Actb gene. Data are presented as mean values. *; P<0.05 by Mann-Whitney U test.

FIG. 2C (left) is a schematic of an experimental protocol and FIG. 2C (right) is a plot of data from C. difficile infected HC-HMA-Rag1^{-/-} mice treated with control or αIL-22 antibody twice before C. difficile inoculation (3 and 5 days prior to infection) and 3 times a week post inoculation. C. difficile load in feces was determined on indicated days post inoculation. Dots represent individual mice. ****; P<0.0001 by 2-way ANOVA with Bonferroni post-hoc test. 20 FIG. 2D shows representative histological images and associated histological scores. Scale bar is 200 μm. Data are presented as mean. *; P<0.05 by Mann-Whitney U test.

FIGS. 3A-3E show that the IL-22 mediated succinate pathway drives the colonization of C. difficile in the healthy microbiota. FIG. 3A is a horizontal bar plot showing data collected from HC-HMA-Rag1^{-/-} mice treated with control or αIL-22 antibody twice (day −5 and day −3) before collecting fecal samples. Bacterial 16S rRNA sequences were analyzed and significantly altered bacterial genera were identified by LEfSe analysis. Enriched bacterial taxa in αIL-22 antibody treated mice are indicated as positive LDA scores (black bars) and enriched bacterial taxa in control antibody treated mice are indicated as negative scores (white bars).

FIG. 3B is a series of bar plots showing amounts of luminal metabolites as analyzed by CE-TOF/MS. Data are presented as mean values (N=13). Dots represent individual mice. *; P<0.05. **; P<0.01 by Mann-Whitney U test.

FIG. 3C is a plot showing data collected from aIL-22 antibody treated HC-HMA-Rag1^{-/-} mice infected with WT JIR8094 or Cd-CD2344-mutant C. difficile. C. difficile load in feces was determined on indicated days post infection. Dots represent individual mice (N=7-8). ****; P<0.0001 by 2-way ANOVA with Bonferroni post-hoc test. N.S.; Not significant.

FIG. 3D is a plot showing data collected from HC-HMA-WT C57BL/6 mice that were administrated with regular water or 1% succinate C. difficile load in feces was determined on indicated days post inoculation. Dots represent individual mice (N=5). ***; P<0.001 by 2-way ANOVA with Bonferroni post-hoc test.

FIG. 3E is a schematic of an experimental protocol (top) and a plot showing data from SPF C57BL/6 mice that were treated with cefoperazon (0.5 mg/ml in drinking water). After 8 days, mice were switched to regular water and allowed to recover for 2 days before being infected with oral gavage with C. difficile VPI 10463 spores (10³ spores/mouse). Mice were inoculated orally with P. faecium JCM30894, P. succinatutens JCM16074 (10⁶ CFU each), or culture medium once before C. difficile inoculation (1 day prior CDI) and 4 times post inoculation (1, 3, 7, and 10 days post CDI) (N=14). The mortality of C. difficile infected mice was assessed. **; P<0.01 by Log-rank test.

FIGS. 4A-4F show that host glycosylation regulated by IL-22 influences the growth of Phascolarctobacterium species. FIG. 4A shows plots of GF mice that were inoculated orally with P. faecium JCM30894 (left) and P. succinatutens JCM16074 (right) (10⁶ CFU each). Mice were then injected with either a mouse IL-22-Fc fusion protein or a control Fc protein. The DNA copy numbers of P. faecium and P. succinatutens in the feces were quantified by qPCR (N=13). The copy number was normalized to that of host genomic DNA (murine Tnf). Data are presented as mean values. Dots represent individual mice. *; P<0.05, ***; P<0.001 by Mann-Whitney U test.

FIG. 4B shows plots of the in vitro growth of P. faecium (left) and P. succinatutens (right) in PYS medium supplemented with colonic mucus derived from control or αIL-22 antibody treated mice (N=9-10). Data are presented as mean values. Dots represent biological replicates. *; P<0.05, ****; P<0.0001 by 1-way ANOVA with Bonferroni post-hoc test.

FIG. 4C shows bar plots of data from P. succinatutens JCM16074 that was cultured in PYS medium supplemented with colonic mucus derived from SPF wild-type C57BL/6 mice. After 48 hours, bacterial RNA was extracted and the mRNA expression of GH33 (left) and GF73 (right) was analyzed by qPCR. Expression was normalized to 16S rRNA levels using a pan-Eubacterial primer. Data are presented as mean values. ****; P<0.0001 by Student's t test.

FIG. 4D is a series of bar plots showing the expression of glycosyltransferase genes in the colonic mucosa of GF Rag1^{-/-} and HC-HMA-Rag1^{-/-} mice injected with control or αIL-22 antibody as determined by qPCR. Expression was normalized to that of the murine Actb gene. Data are presented as mean±s.d. *; P<0.05, **; P<0.01 by 1-way ANOVA with Bonferroni post-hoc test. N.S.; Not significant.

FIG. 4E shows bar plots of data from experiments in which colonic mucus derived from control or αIL-22 antibody treated HMA-Rag1^{-/-} mice were separated into GuHCl-soluble and GuHCl-insoluble fractions. N-glycans present in the insoluble and insoluble fractions were analyzed by liquid chromatograph-electrospray ionization tandem mass spectrometry. Data are presented as mean±s.d. (N=5). **; P<0.01 by Mann-Whitney U test.

FIG. 4F is a series of bar plots showing the relative intensity of high-mannnose, hybrid, or complex N-glycans in soluble and insoluble fractions. Data are presented as mean values Dots represent individual mice. *; P<0.05, **; P<0.01 by Mann-Whitney U test.

FIGS. 5A-5D show N-glycan related glycosyltranferase gene expression and CDI risk in UC patients. FIG. 5A is a series of plots quantifying mRNA expression of glycosyltransferase-related genes in the colonic tissue from control subjects (N=11), patients with inactive UC (N=23), and patients with active UC (N=74). Data were derived from Gene Expression Omnibus (GEO) data set GSE75214. Dots represent individual subjects. *; P<0.05, **; P<0.01, ****; P<0.0001 by Kruskal-Wallis test with Dunn's post-hoc test. N.S.; Not significant.

FIG. 5B is a series of plots showing the correlation between MGAT4A/MGAT4B and Il22RA2 mRNA expression in 3 groups. Statistical significance was measured by Pearson's correlation test.

FIG. 5C shows plots of data collected in experiments in which GF WT C57BL/6 mice were colonized with the gut microbiotas obtained from UC patients for 2 weeks. UC-HMA mice were then infected with C. difficile VPI 10463 spores (10³ spores/mouse). C. difficile load in feces was determined on indicated days post infection (left panel). The mortality of C. difficile infected UC-HMA mice was also recorded (right panel). Dots represent individual mice.

FIG. 5D shows representative histological images and associated histological scores. Scale bar is 100 μm. Dots represent individual mice.

FIGS. 6A-6C show that C. difficile utilizes luminal succinate for its growth in UC patients. FIG. 6A is a bar plot showing the ex vivo germination and growth of C. difficile strain VPI 10463 in cecum contents isolated from HC-HMA and UC-HMA mice. *; P<0.05 by 1-way ANOVA with Bonferroni post-hoc test. N.S.; Not significant.

FIG. 6B shows a schematic of an experimental protocol and data for ex vivo germination and growth of wild-type or Cd-CD2344-mutant C. difficile in UC luminal contents. ****; P<0.0001 by 2-way ANOVA with Bonferroni post-hoc test.

FIG. 6C shows a schematic of an experimental protocol and data from experiments in which UC-HMA mice were infected with wild-type or Cd-CD2344-mutant C. difficile. C. difficile load in feces was determined on indicated days post infection. Dots represent individual mice. *; P<0.05 by 2-way ANOVA with Bonferroni post-hoc test. N.S.; Not significant.

FIGS. 7A-7J show that restoration of luminal metabolites reduces the risk for C. difficile infection. FIG. 7A is a schematic of an experimental protocol in which UC-HMA mice were infected with C. difficile VPI 10463 spores (10³ spores/mouse) and transplanted with a healthy human-derived fecal microbiota. A one-dose FMT (FMT (1)) was administered on day −3 CDI. A five-dose FMT (FMT (5)) was administered on day −3, 1, 3, 7, and 10 CDI). Feces were collected for microbiota and metabolite analysis.

FIG. 7B is a plot showing the C. difficile load in feces determined on indicated days post infection. Dots represent individual mice. *, †; P<0.05, **; P<0.01, ****, ††††; P<0.0001 by 2-way ANOVA with Bonferroni post-hoc test.

FIG. 7C is a plot of data from experiments in which fecal microbiota before CDI (day 0) was harvested and bacterial 16S rRNA sequences were analyzed. Microbial community structures were analyzed using the Yue and Clayton dissimilarity distance metric (θYC) and are shown in a nonmetric multidimensional scaling plot.

FIG. 7D is a series of bar plots of the Shannon index (α-diversity, left) and number of OTUs (richness, right) of UC-HMA or FMT-treated UC-HMA mice before CDI (day 0). Data are presented as mean values. Dots represent individual mice. **; P<0.01 by Mann-Whitney U test.

FIG. 7E is an abundance plot showing bacterial-taxon-based analysis at the family level in the feces before CDI (day 0).

FIG. 7F is a plot showing the relative abundance of Phascolarctobacterium in the feces before CDI (day 0). Data are presented as mean values. Dots represent individual mice. **; P<0.01 by Mann-Whitney U test.

FIG. 7G is a plot showing the fecal succinate concentration at day 0 analyzed by CE-TOF/MS. Data are presented as mean values. Dots represent individual mice. *; P<0.01 by Student t test.

FIG. 7H is a schematic of an experimental protocol in which UC-HMA mice were infected with C. difficile VPI 10463 spores (10³ spores/mouse). Mice were inoculated with P. faecium JCM30894 and P. succinatutens JCM16074 (106 CFU each) once before C. difficile inoculation (3 days prior CDI) and 2 times post inoculation (1 day and 3 days post CDI). Feces were collected before CDI (day 0) for luminal succinate measurement and day 1 and day 3 post CDI for the titration of C. difficile load in feces.

FIG. 7I is a plot showing the luminal succinate concentration in UC-HMA mice transplanted with or without Phascolarctobacterium spp. before CDI (at day 0). Luminal succinate was analyzed by LC-MS. Data are presented as mean values. ***; P<0.001 by Mann-Whitney U test.

FIG. 7J is a plot showing the C. difficile load in feces determined on indicated days post infection. Dots represent individual mice (N=15). *; P<0.05, **; P<0.01 by 2-way ANOVA with Bonferroni post-hoc test.

FIG. 8 is a series of plots showing that IL-22 neutralization inhibits the expression of antimicrobial proteins. Reg3b and Reg3g mRNA levels in control or anti-IL-22 antibody treated HC-HMA Rag1^{-/-} mice were determined by qPCR (N=8). Expression was normalized to that of the murine Actb gene. Data are presented as mean values. *; P<0.05 by Mann-Whitney U test.

FIGS. 9A-9C show that IL-22 shapes gut microbial community. HC-HMA-Rag1^{-/-} mice were treated with control or αIL-22 antibody twice (day −5 and day −3) before collecting fecal samples. Bacterial 16S rRNA sequences were analyzed. FIG. 9A shows plots of the Shannon index (α-diversity, top) and number of OTUs (richness, bottom) of control or αIL-22 antibody treated HMA-Rag1^{-/-} mice. Data are presented as mean values. Dots represent individual mice. N.S.; Not significant by Mann-Whitney U test.

FIG. 9B is a plot showing microbial community structures analyzed using the Yue and Clayton dissimilarity distance metric (θYC) and shown in a nonmetric multidimensional scaling plot.

FIG. 9C is an abundance plot showing a bacterial-taxon-based analysis at the family level in the feces.

FIGS. 10A-10C show data from luminal metabolomic analysis in IL-22 neutralized HMA mice. FIG. 10A shows a principal component analysis of metabolome data. The ellipse denotes 95% significance limit of the model as defined by Hotelling t test.

FIG. 10B is a loading scatter plot of the principal component analysis.

FIG. 10C is a series of plots showing amounts of luminal metabolites analyzed by CE-TOF/MS. Data are presented as mean values (N=3). *; P<0.05. by Student's t test N.S., not significant.

FIG. 11 is a series of plots showing C. difficile growth on succinate. In vitro growth of wild-type JIR8094 (open circles) or Cd-CD2344-mutant (closed circles) C. difficile in a minimal medium supplemented with glucose (left plot) or succinate (right plot). ***; P<0.001, ****; P<0.0001 by 2-way ANOVA with Bonferroni post-hoc test.

FIG. 12A-12B show that succinate is not required for the growth of C. difficile in germ-free mice. FIG. 12A is a plot of data collected from GF C57BL/6 mice infected with wild-type JIR8094 or Cd-CD2344-mutant C. difficile strains. C. difficile load in feces was determined on indicated days post infection. Dots represent individual mice. N.S.; Not significant by 2-way ANOVA with Bonferroni post-hoc test.

FIG. 12B is a plot showing the mortality of C. difficile infected mice.

FIG. 13A-13E show gene expression profiles in UC patient cohorts. FIG. 13A is a plot of mRNA expression of IL22 mRNA in the colonic tissue from control subjects, patients with inactive UC, and patients with active UC. Data were derived from GEO data set GSE75214. Dots represent individual subjects. Data are presented as mean values. *; P<0.05, ****; P<0.0001 by Kruskal-Wallis test with Dunn's posttest. N.S., not significant.

FIGS. 13B and 13D show expression of IL-22, IL-22 BP, MGAT4a, and MGAT4b mRNA in the colonic tissue from control subjects and patients with active UC. Data were derived from GEO data sets GSE16870 and GSE73661 as indicated on the plots.

FIGS. 13C and 13E are plots showing the correlation between MGAT4a and MGAT4b with IL-22BP mRNA expression in 3 groups. Statistical significance was measured by Pearson's correlation test. Dots represent individual subjects. Data were derived from GEO data sets GSE16870 and GSE73661 as indicated on the plots. Data are presented as mean values. *; P<0.05, **; P<0.01 ***; P<0.001 ****; P<0.0001 Mann-Whitney U test. N.S., not significant (FIGS. 13B and 13D). Statistical significance was measured by Pearson's correlation test (FIGS. 13C and 13E).

FIG. 14 is a plot showing that FMT restores the microbial composition in UC-HMA mice. Significantly altered bacteria in pre-C. difficile infected UC-HMA mice with or without FMT were identified by LEfSe analysis. UC-HMA mice-enriched taxa have a positive LDA score (red and extending to the left), and FMT-treated UC-HMA mice-enriched taxa have a negative score (green and extending to the right).

FIGS. 15A-15C show luminal metabolomic analysis in FMT-treated UC-HMA mice. FIG. 15A is a plot showing principal component analysis of metabolome data. The ellipse denotes 95% significance limit of the model, as defined by Hotelling t test.

FIG. 15B is a loading scatter plot of the principal component analysis.

FIG. 15C is a series of plots from measurements of luminal metabolites analyzed by CE-TOF/MS. Data are presented as mean (N=3). *; P<0.05. **; P<0.01, ***; P<0.001 by Student's t test N.S., not significant.

FIG. 16 shows that Phascolarctobacterium inoculation protects mice from CDI. SPF C57BL/6 mice were treated with cefoperazon (0.5 mg/ml in drinking water). After 8 days, the mice were switched to regular water and allowed to recover for 2 days before being infected with C. difficile VPI spores. Mice were treated with P. faecium JCM30894 and P. succinatutens JCM16074 (10⁶ CFU each) or culture medium by oral gavage once before C. difficile inoculation (1 day prior to CDI, pre-treatment) or 4 times post inoculation (1, 3, 7, and 10 days post infection, post-treatment) (N=5). The mortality of C. difficile infected mice was assessed. *, †; P<0.05 by Log-rank test.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to preventing and treating gastrointestinal dysbiosis and particularly, but not exclusively, to compositions, methods, systems, and kits for treating and/or preventing Clostridioides difficile infection in an organism.

The gut resident microbiota plays a role in the prevention of CDI. For example, metabolites (e.g., secondary bile acids) produced by the resident microbiota directly inhibit C. difficile colonization in the intestine. On the other hand, the involvement of host immunity in the microbiota-conferred colonization resistance remains poorly understood. Experiments conducted during the development of the technology provided herein indicated that interleukin (IL)-22 is induced by colonization of the gut microbiota and prevents CDI in human microbiota-associated (HMA) mice. IL-22 signaling in HMA mice increased host glycosylation, thus fostering the growth of succinate consuming Phascolarctobacterium spp. within the gut microbiota. Phascolarctobacterium spp. reduced the availability of luminal succinate, a metabolite utilized by C. difficile for growth in the intestine. Moreover, IL-22-regulated host glycosylation is likely impaired in patients with ulcerative colitis (UC). The expression of N-glycosylation-related enzymes, MGAT4A and MGAT4B, was reduced in UC patients and was inversely correlated with that of IL22RA2, a soluble inhibitory IL-22 receptor. Consistently, mice colonized with UC patient-derived microbiotas were susceptible to CDI. Transplantation of healthy human-derived microbiotas or Phascolarctobacterium spp. reduced luminal succinate levels and restored colonization resistance in UC-HMA mice. Thus, IL-22-mediated host glycosylation fosters the growth of commensal bacteria that compete with C. difficile for the nutritional niche in the gut.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present or “absent”) is used in a relative sense to describe the amount or level of a particular entity (e.g., a nucleic acid (e.g., an RNA (e.g., a mRNA)), a microorganism (e.g., Phascolarctobacterium spp. (e.g., P. faecium and/or P. succinatutens), C. difficile), a protein (e.g., IL-22), or other biomolecule or organism discussed herein and/or known in the art). For example, when an organism, protein, and/or nucleic acid is said to be “present” in a test sample, it means the level or amount of this organism, protein, and/or nucleic acid is above a pre-determined threshold; conversely, when an organism, protein, and/or nucleic acid is said to be “absent” in a test sample, it means the level or amount of this nucleic acid is below a pre-determined threshold. The pre-determined threshold may be the threshold for detectability associated with the particular test used to detect the organism, protein, and/or nucleic acid or any other threshold. When an organism, protein, and/or nucleic acid is “detected” in a sample it is “present” in the sample; when an organism, protein, and/or nucleic acid is “not detected” it is “absent” from the sample. Further, a sample in which an organism, protein, and/or nucleic acid is “detected” or in which the organism, protein, and/or nucleic acid is “present” is a sample that is “positive” for the organism, protein, and/or nucleic acid. A sample in which an organism, protein, and/or nucleic acid is “not detected” or in which the organism, protein, and/or nucleic acid is “absent” is a sample that is “negative” for the organism, protein, and/or nucleic acid.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods.

As used herein, the terms “bacteria” and “bacterium” refer to prokaryotic organisms of the domain Bacteria in the three-domain system (see Woese C R, et al., Proc Natl Acad Sci USA 1990, 87: 4576-79, incorporated herein by reference). It is intended that the terms encompass all microorganisms considered to be bacteria including Mycobacterium, Mycoplasma, Chlamydia, Actinomyces, Streptomyces, and Rickettsia. All forms of bacteria are included within this definition including cocci, bacilli, spirochetes, spheroplasts, protoplasts, etc. In some embodiments, bacteria are capable of causing disease and product degradation or spoilage.

As used herein, a “pathogen” is an organism or agent that is capable of causing a disease. The terms “non-pathogenic microbe” or “non-pathogenic microorganism” include all known and unknown non-pathogenic microbes (Bacteria, Archaea, and/or Eukarya) and any pathogenic microbe that has been mutated or converted to a non-pathogenic state. Furthermore, a skilled artisan recognizes that some microbes may be pathogenic to specific species and non-pathogenic to other species; thus, these microbes can be utilized in the species in which it is non-pathogenic or mutated so that it is non-pathogenic.

As used herein, the term “strain” when used in reference to a microorganism describes an isolate of a microorganism (e.g., bacteria, virus, fungus, parasite) considered to be of the same species but with a unique genome and, if nucleotide changes are non-synonymous, a unique proteome differing from other strains of the same organism. Strains may differ in their non-chromosomal genetic complement. Typically, strains are the result of isolation from a different host or at a different location and time, but multiple strains of the same organism may be isolated from the same host.

As used herein, the term “infection” refers to the invasion of a host animal by pathogenic microorganisms such as bacteria. For example, the infection may include the excessive growth of microorganisms that are normally present in or on the body of an animal or growth of microorganisms that are not normally present in or on the animal. More generally, an infection can be any situation in which the presence of a microorganism population(s) is damaging to a host animal. Thus, an animal is “suffering” from an infection when an excessive amount of a bacterial population is present in or on the animal's body, or when the presence of a microorganism population(s) is damaging the cells or other tissue of the animal.

As used herein, the term “subject” refers to individuals (e.g., human, animal, or another organism) to be treated by the methods or compositions of the present technology. Subjects include, but are not limited to, mammals (e.g., murines, simians, equines, bovines, porcines, canines, felines, and the like), and most preferably includes humans. In the context of the technology described herein, the term “subject” generally refers to an individual who will receive or who has received treatment for a condition characterized by the presence of an infectious microbe, or in anticipation of possible exposure to an infectious microbe. The term “mammal” is defined as an individual belonging to the class Mammalia and includes, without limitation, humans, domestic, and farm animals; and zoo, sports, or pet animals, such as sheep, dogs, horses, cats, or cows. In some embodiments, the subject is a mouse or rat. In some embodiments, the subject is a human.

As used herein, the term “diagnosed” refers to identifying and/or recognizing a disease (e.g., an infection) and/or a subject having a disease by its signs and symptoms, or genetic analysis, pathological analysis, histological analysis, and the like.

As used herein, the term “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reactions that occur within a natural environment.

As used herein, the term “virulence” refers to the degree of pathogenicity of a microorganism, e.g., as indicated by the severity of the disease produced or its ability to invade the tissues of a subject. It is generally measured experimentally by the median lethal dose (LD50) or median infective dose (ID50). The term may also be used to refer to the competence of any infectious agent to produce pathologic effects.

As used herein, the term “effective amount” refers to the amount of a composition sufficient to effect beneficial or desired results. An effective amount can be administered in one or more administrations, applications, or dosages and is not intended to be limited to a particular formulation or administration route.

As used herein, the terms “administration” and “administering” refer to the act of giving a drug, prodrug, or other agent, or therapeutic treatment (e.g., compositions of the present technology) to a physiological system (e.g., a subject or in vivo, in vitro, or ex vivo cells, tissues, and organs). Exemplary routes of administration to the human body are through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.), fecal transplant, and the like.

As used herein, the term “co-administration” refers to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration is readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent.

As used herein, the term “pharmaceutical composition” refers to the combination of an active agent with a carrier, inert or active, making the composition especially suitable for diagnostic or therapeutic use in vitro, in vivo, or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline, water, emulsions (e.g., such as an oil/water or water/oil emulsion), and various types of wetting agents, any and all solvents, dispersion media, coatings, sodium lauryl sulfate, isotonic and absorption delaying agents, disintegrants (e.g., potato starch or sodium starch glycolate), and the like. In some embodiments, the compositions include stabilizers and preservatives. For examples of carriers, stabilizers, and adjuvants, see, e.g., Martin, Remington's Pharmaceutical Sciences, 15th Ed., Mack Publ. Co. (Easton, Pa., 1975), incorporated herein by reference).

As used herein, the term “pharmaceutically acceptable salt” refers to any salt (e.g., obtained by reaction with an acid or a base) of a compound of the present technology that is physiologically tolerated in the target subject (e.g., a mammalian subject and/or in vivo or ex vivo, cells, tissues, or organs). Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. For therapeutic use, salts are contemplated as being pharmaceutically acceptable. However, salts of acids and bases that are non-pharmaceutically acceptable may also find use, for example, in the preparation or purification of a pharmaceutically acceptable compound.

As used herein, the term “therapeutic agent,” refers to compositions that decrease the infectivity, morbidity, or onset of mortality in a subject contacted by an infectious or pathogenic microbe or that prevent infectivity, morbidity, or onset of mortality in a host contacted by an infectious or pathogenic microbe. As used herein, therapeutic agents encompass agents used prophylactically, e.g., in the absence of an infectious or pathogenic microbe, in view of possible future exposure to an infectious or pathogenic microbe. Such agents may additionally comprise pharmaceutically acceptable compounds (e.g., adjuvants, excipients, stabilizers, diluents, and the like). In some embodiments, the therapeutic agents of the present technology are administered in the form of topical compositions, injectable compositions, ingestible compositions, rectally-administered compositions, transplantable compositions, and the like.

As used herein, the term “treatment” refers to an intervention (e.g., a clinical intervention) made in response to a disease, disorder, or physiological condition manifested by a patient. The aim of treatment may include, but is not limited to, one or more of alleviating symptoms; preventing symptoms; slowing the progression or worsening of a disease, disorder, or condition; stopping the progression or worsening of a disease, disorder, or condition; slowing the remission of a disease, disorder, or condition; and/or stopping the remission of a disease, disorder, or condition. In some embodiments, “treatment” refers to both therapeutic treatment and prophylactic or preventative measures. Those in need of treatment include those already affected by a disease or disorder or undesired physiological condition and those in which the disease or disorder or undesired physiological condition is to be prevented. For example, in some embodiments, the treatment may reduce, alleviate, or eradicate the symptom(s) of the disease(s).

As used herein, the term “prevention” refers to any activity that reduces the burden on an individual later having CDI. This can take place at primary, secondary, and/or tertiary prevention levels, wherein: a) primary prevention avoids the development of symptoms, a disorder, and/or a condition; b) secondary prevention activities are aimed at early stages of the condition, disorder, and/or symptom treatment, thereby increasing opportunities for interventions to prevent progression of the condition, disorder, and/or symptom and emergence of symptoms; and c) tertiary prevention reduces the negative impact of an already established condition, disorder, symptom by, for example, restoring function and/or reducing any condition, disorder, symptom or related complications.

As used herein, the term “probiotic” refers to live microorganisms, which, when administered in adequate amounts, confer a health benefit on the host. The probiotics may be available in foods and dietary supplements (for example, but not limited to capsules, tablets, and powders). Non-limiting examples of foods containing probiotic include dairy products such as yogurt, fermented and unfermented milk, smoothies, butter, cream, hummus, kombucha, salad dressing, miso, tempeh, nutrition bars, and some juices and soy beverages.

As used herein, the term “metabolite” refers to any molecule involved in metabolism. Metabolites can be products, substrates, or intermediates in metabolic processes. For example, the metabolite can be a primary metabolite, a secondary metabolite, an organic metabolite, or an inorganic metabolite. Metabolites include, without limitation, fatty acids, amino acids, peptides, acylcarnitines, monosaccharides, oligosaccharides, lipids and phospholipids, prostaglandins, hydroxyeicosatetraenoic acids, hydroxyoctadecadienoic acids, steroids, bile acids, glycolipids, and phospholipids. In some embodiments, the metabolite is a microbial metabolite that is a metabolite produced by a microbe to, for example, regulate its own growth and development, to encourage beneficial interaction with other organisms, and to suppress organisms that are harmful to it. In some embodiments, the metabolite is produced by the gut of a subject to encourage beneficial interaction with other organisms and/or to suppress organisms that are harmful to the subject. The microbial metabolites can be, for example, small molecular weight compounds (e.g., <2,500 Da). In some embodiments, the metabolite is an analogue of a microbial metabolite. In some embodiments, the microbial metabolites and analogues thereof include short-chain fatty acids (SCFAs), medium-chain fatty acids, and long-chain fatty acids; and salts and esters of the short-, medium-and long-fatty acids. Non-limiting examples of fatty acids include SCFAs acetate, propionate, and butyrate.

As used herein, the term “antibody” includes polyclonal antibodies, monoclonal antibodies (including full length antibodies that have an immunoglobulin Fc region), antibody compositions with polyepitopic specificity, multispecific antibodies (e.g., bispecific antibodies, diabodies, and single-chain molecules, and antibody fragments (e.g., Fab or F(ab′)2, and Fv). For the structure and properties of the different classes of antibodies see, e.g., Basic and Clinical Immunology, 8th Edition, Daniel P. Sties, Abba I. Terr and Tristram G. Parsolw (eds), Appleton & Lange, Norwalk, Conn., 1994.

As used herein in, a “normal level” of gut microbiota or a member of the gut microbiota refers to a level of gut microbiota or a member of the gut microbiota in healthy subjects. One of skill in the art will appreciate that variability in the composition of gut microbiota may exist between healthy individuals and a normal level can be established as a representative of the composition of gut microbiota in healthy individuals of a population, or a population of healthy subjects, for the comparison. Various criteria can be used to determine the inclusion and/or exclusion of a particular subject in the reference population, including but not limited to age of the subject (e.g., the reference subject can be within the same age group as the subject in need of treatment) and gender of the subject (e.g., the reference subject can be the same gender as the subject in need of treatment).

As used herein, the term “microbiota” refers to the communities of microbes that live in or on an organism, both sustainably and transiently, including eukaryotes, archaea, bacteria, and viruses (including bacterial viruses (e.g., phage)). The microbiota consumes, stores, and re-distributes energy and nutrients, and it mediates physiologically important chemical transformations.

As used herein, the term “microbiome” refers to the genetic content of the communities of microbes that live in and on an organism (e.g., a human), both sustainably and transiently, including eukaryotes, archaea, bacteria, and viruses (including bacterial viruses (e.g., phage)), wherein “genetic content” includes genomic DNA, RNA such as micro RNA and ribosomal RNA, the epigenome, plasmids, and all other types of genetic information.

As used herein, the term “dysbiosis” refers to a state of the microbiota or microbiome of the gut or other body area, including mucosal or skin surfaces, in which the normal diversity and/or function of the ecological network is disrupted. Any disruption from the preferred (e.g., ideal) state of the microbiota can be considered a dysbiosis, even if such dysbiosis does not result in a detectable decrease in health. This state of dysbiosis may be unhealthy, it may be unhealthy under only certain conditions, or it may prevent a subject from becoming healthier. Dysbiosis may be due to a decrease in diversity, the overgrowth of one or more pathogens or pathobionts, symbiotic organisms able to cause disease only when certain genetic and/or environmental conditions are present in a patient, or the shift to an ecological network that no longer provides a beneficial function to the host and therefore no longer promotes health.

As used herein, the term “isolated” refers to a bacterium or other entity or substance that has been (1) separated from at least some of the components with which it was associated when initially produced (whether in nature or in an experimental setting); and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or more of the other components with which they were initially associated. In some embodiments, isolated bacteria are more than about 80%, about 85%, about 90%, about 91%, about 92%, about 93%, about 94%, about 95%, about 96%, about 97%, about 98%, about 99%, or more than about 99% pure.

As used herein, a substance is “pure” if it is substantially free of other components. The terms “purify”, “purifying”, and “purified” refer to a bacterium or other material that has been separated from at least some of the components with which it was associated either when initially produced or generated (e.g., whether in nature or in an experimental setting) or during any time after its initial production. A bacterium or a bacterial population may be considered purified if it is isolated at or after production, such as from a material or environment containing the bacterium or bacterial population, and a purified bacterium or bacterial population may contain other materials up to about 10%, about 20%, about 30%, about 40%, about 50%, about 60%, about 70%, about 80%, about 90%, or above about 90% and still be considered “isolated.” In some embodiments, purified bacteria and bacterial populations are more than about 80%, about 85%, about 90%, about 91%, about 92%, about 93%, about 94%, about 95%, about 96%, about 97%, about 98%, about 99%, or more than about 99% pure. In the instance of bacterial compositions provided herein, the one or more bacterial types present in the composition can be independently purified from one or more other bacteria produced and/or present in the material or environment containing the bacterial type.

As used herein, the term “colonization” (e.g., when referring to a host organism) refers to the non-transitory residence of a bacterium or other microscopic organism in, or, or with the host. As used herein, “reducing colonization” of a host subject gastrointestinal tract (or any other microbiotal niche) by a pathogenic bacterium includes a reduction in the residence time of the pathogen in the gastrointestinal tract and/or a reduction in the number (or concentration) of the pathogen in the gastrointestinal tract or adhered to the luminal surface of the gastrointestinal tract. Measuring reductions of adherent pathogens may be demonstrated, e.g., by a biopsy sample, or reductions may be measured indirectly, e.g., by measuring the pathogenic burden in the stool of a mammalian host.

As used herein, the term “combination” of two or more bacteria includes the physical co-existence of the two bacteria, either in the same material or product or in physically connected products, as well as the temporal co-administration or co-localization of the two bacteria.

As used herein, the term “operational taxonomic unit” (“OTU”) refers to a terminal leaf in a phylogenetic tree and is defined by a specific genetic sequence and all sequences that share sequence identity to this sequence at the level of species. A “type” or a plurality of “types” of bacteria includes an OTU or a plurality of different OTUs, and encompasses a strain, species, genus, family, or order of bacteria. The specific genetic sequence may be the 16S rRNA sequence or a portion of the 16S rRNA sequence or it may be a functionally conserved housekeeping gene found broadly across the eubacterial kingdom. OTUs share at least 95%, 96%, 97%, 98%, or 99% sequence identity. OTUs are frequently defined by comparing sequences between organisms. Sequences with less than 95% sequence identity are not considered to form part of the same OTU. In some embodiments, OTUs are defined either by full 16S sequencing of the rRNA gene, by sequencing of a specific hypervariable region of this gene (e.g., V1, V2, V3, V4, V5, V6, V7, V8, or V9), or by sequencing of any combination of hypervariable regions from this gene (e.g., V1-3 or V3-5). The bacterial 16S rDNA is approximately 1500 nucleotides in length and is used in reconstructing the evolutionary relationships and sequence similarity of one bacterial isolate to another using phylogenetic approaches. 16S sequences are used for phylogenetic reconstruction because they are highly conserved, but contain specific hypervariable regions that harbor sufficient nucleotide diversity to differentiate taxa (e.g., genera and species) of most microbes. Using well known techniques, to determine the full 16S sequence or the sequence of any hypervariable region of the 16S sequence, genomic DNA is extracted from a bacterial sample, the 16S rDNA (full region or specific hypervariable regions) is amplified using polymerase chain reaction (PCR), the PCR products cleaned, and nucleotide sequences examined. The technology is not limited in the sequencing technology that is used and includes, e.g., Sanger sequencing, dye terminator sequencing, sequencing by synthesis, and single molecule sequencing. Illustrative non-limiting examples of nucleic acid sequencing techniques include, but are not limited to, single molecule real time sequencing (Pacific Biosciences), sequencing by synthesis (Illumina, Inc.), 454 pyrosequencing (Roche Diagnostics, Inc.), SOLiD sequencing (Life Technologies, Inc.), ion semiconductor sequencing (Life Technologies, Inc.), and sequencing using a nanopore.

### Interleukin-22

Interleukin (IL)-22 is a member of the IL-10 family of cytokines and is produced by various immune cells. Recent accumulating evidence has suggested that IL-22 plays a role in mucosal defense mechanisms employed by the gastrointestinal tract (11, 12). IL-22 promotes epithelial barrier integrity, elicits the secretion of antimicrobial peptides (e.g., RegIII proteins and defensins), and induces iron scavengers, thereby limiting the growth of pathogens and dampening pathogen-induced mucosal damage (13, 14, 15, 16). Moreover, IL-22 increases host epithelial glycosylation, which, in turn, prevents the growth of enteric pathogens, such as Citrobacter rodentium and Salmonella (17, 18). In the gut, IL-22 production is regulated by the resident microbiota (19, 20, 21, 22), indicating that IL-22 induction is used by the gut microbiota to mediate colonization resistance against enteric pathogens. Previous studies have suggested that IL-22 is not required for prevention of C. difficile growth in the intestine (23, 24, 25). However, in those studies, the intestinal microbiota was depleted by treatment with antibiotics prior to CDI (23, 24, 25), and therefore, microbiota-dependent induction of IL-22 would not have been detected in previous models. Thus, the involvement of the microbiota-induced IL-22 pathway and the contribution of defects in the IL-22 pathway to the increased risk of CDI remain largely unknown.

During the development of embodiments of the technology provided herein, experiments were conducted in which IL-22 was induced by colonization of the gut microbiota and was necessary for preventing CDI in human microbiota-associated (HMA) mice. In these experiments, colonization of germ-free (GF) mice with human microbiotas elevated the levels of mucosal IL-22, which in turn increased host glycosylation. Increased host glycosylation subsequently reshaped the composition of the gut microbiota and altered its carbohydrate metabolism. As expected, a blockade of IL-22 signaling influenced the gut microbial metabolism and led to the accumulation of succinate, which in turn promoted the growth of C. difficile. In contrast, a dysbiotic microbiota (e.g., resulting from impaired IL-22 signaling) produces a gut nutrient environment that promotes the opportunistic growth of pathogens, such as C. difficile. Once the normal metabolic function of the microbiota was restored, the gut microbiota protected the host against enteric pathogens. Thus, IL-22-mediated host glycosylation fosters the growth of commensal bacteria that compete with C. difficile for the nutritional niche.

IL-22 is a member of the IL-10 cytokine family, which includes IL-19, IL-20, IL-24, and IL-26. This cytokine family mediates cellular inflammatory responses. IL-22 signals through the interferon receptor-related proteins CRF2-4 and IL-22R. IL-22 forms cell surface complexes with IL-22R1 and IL-10R2 receptor chains, which subsequently produces signal transduction through the receptor IL-10R2. The IL-22/IL-22R1/IL-10R2 complex activates intracellular kinases (e.g., JAK1, Tyk2, and MAP kinases) and transcription factors, especially STAT3. IL-22 can induce IL-20 and IL-24 signaling when IL-22R1 pairs with IL-20R2. IL-22 shares use of the IL-10R2 receptor in cell signaling with other members of this family (e.g., IL-10, IL-26, IL-28A/B and IL-29).

In humans, the interleukin-22 (IL-22) protein is encoded by the IL22 gene located at chromosome 12q15. The IL22 gene encodes a 179-amino acid protein that shares 79% identity with the mouse homolog. IL-22 is produced by several types of human immune cells including αβ T cells of classes Th1, Th22, and Th17, and by _{Y}δ cells, NKT, innate lymphoid cells, neutrophils, and macrophages. IL-22 binds to a cell-surface complex comprising IL-22R1 and IL-10R2 receptor chains and a soluble binding protein, IL-22BP, which shares sequence similarity with an extracellular region of IL-22R1 (sIL-22R1). IL-22 targets non-hematopoietic cells in the epithelium and stroma of the liver, lung, skin, thymus, pancreas, kidney, gastrointestinal tract, synovial tissues, heart, breast, eye, and adipose tissue. Production of IL-22 is induced mainly by IL-23 receptor signaling and is known to be induced also by IL-1β. IL-23 is produced by dendritic cells after recognition of ligands by specific Toll-like receptors. See, e.g., Dudakov (2015) “Interleukin-22: immunology and pathology” Annu Rev Immunol. 33: 747-85, incorporated herein by reference.

### Glycans

Accumulating evidence shows that glycan metabolism affects the composition of the gut microbial ecosystem. The gut commensal microbiota and certain enteric pathogens can utilize host glycans, such as mucus glycans, as a source of carbon and energy (6). IL-22 promotes the expression of host glycosyltransferases, such as Mgat4a and Mgat4b. Since these enzymes catalyze the transfer of N-acetylglucosamine (GlcNAc) to the core mannose residues (41, 42), upregulation of these genes produces elevated N-glycosylation in host cells and mucus proteins. Increased host glycosylation feeds specific populations of gut commensal bacteria that utilize host glycan for their growth. Consistently, data collected during experiments conducted during the development of embodiments described herein indicated that N-glycosylation in both host cells (soluble fraction) and mucus proteins (insoluble fraction) was compromised in the IL-22-neutralized mice compared to control mice, although the overall abundance of GlcNAc seemed unchanged. This indicated that the transfer of GlcNAc to N-linked glycan might be impaired only in subsets of host-derived proteins (e.g., epithelial surface proteins) but it is sufficient to limit the growth of some commensal bacteria that rely on host N-linked glycans.

Accordingly, in some embodiments, the technology relates to modulating (e.g., increasing, decreasing, and/or modifying the composition of) glycan production and/or glycan concentration in the gut lumen. In some embodiments, the technology relates to increasing the expression and/or activity of the Mgat4a and/or Mgat4b genes and/or gene products of the Mgat4a and/or Mgat4b genes (e.g., MGAT4A, MGAT4B) or a homolog thereof.

### Microorganisms

During the development of embodiments of the technology provided herein, experiments produced data indicating that expression of host glycosyltransferases correlates with the abundance of Phascolarctobacterium. Phascolarctobacterium spp. (phylum Firmicutes, family Acidaminococcaceae), such as P. faecium and P. succinatutens, are obligate anaerobic, Gram-negative, non-spore-forming and non-motile bacteria that abundantly colonize the human gastrointestinal tract (30, 43). P. faecium colonizes the human gut under normal conditions at a rate of approximately 3.22-5.76 log cells g⁻¹ gut contents for infants (e.g., less than 1 year old) and at a rate of approximately 3.06-9.33 log cells g⁻¹ for humans aged 1 to 10. Humans aged 10 to 30 years old have P. faecium in the gut under normal conditions at a rate of approximately 4.20-9.26 log cells g⁻¹ gut contents; humans aged 30 to 60 years old have P. faecium in the gut under normal conditions at a rate of approximately 3.86-9.03 log cells g⁻¹ gut contents; and humans aged more than 60 years old have P. faecium in the gut under normal conditions at a rate of approximately 3.18-9.37 log cells g⁻¹ gut contents (43). These numbers of P. faecium cells in the human gut are a relative abundance of approximately 0.004 to 1.479 as a permillage of P. faecium in total gut bacteria.

Given the abundance of this genus in the human gut and its ability to generate short chain fatty acids (SCFA), such as acetate and propionate (30), Phascolarctobacterium spp. may contribute to the regulation of gut homeostasis. The abundance of Phascolarctobacterium is markedly reduced in CDI patients compared to healthy control subjects (44) and the abundance of Phascolarctobacterium was restored in CDI patients following FMT, which ameliorated clinical symptoms (45). Thus, the abundance of Phascolarctobacterium is likely involved in host protection against CDI.

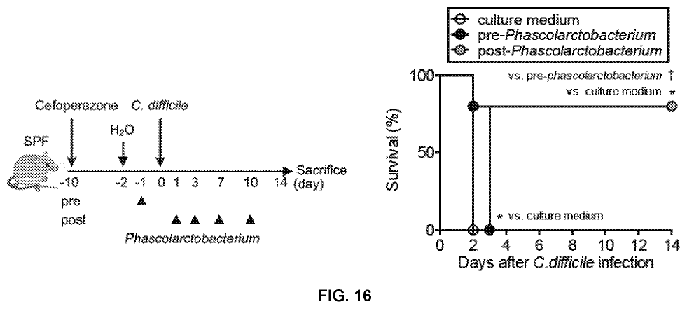

However, the mechanisms by which Phascolarctobacterium spp. are regulated within the gut microbiota were poorly understood. Phascolarctobacterium spp. utilize a narrow spectrum of carbohydrates and the growth of Phascolarctobacterium relies almost solely on succinate (30). During the development of embodiments of the technology provided herein, analysis of genomic sequence indicated that Phascolarctobacterium spp. have nucleic acids that encode glycoside hydrolases associated with host glycan degradation. Hence, it is reasonable to conclude that Phascolarctobacterium spp. forage host glycans. Therefore, IL-22-mediated induction of host N-glycosylation likely promotes the growth of this bacterial genus in the gut. The increased abundance of Phascolarctobacterium spp. leads to a more efficient consumption of succinate in the gut lumen, which limits the opportunistic growth of C. difficile on the succinate food source (32). Indeed, administration of Phascolarctobacterium spp. sufficiently protected antibiotic-treated mice from CDI. Importantly, Phascolarctobacterium improved the mortality of infected animals after the colonization by C. difficile (FIG. 16). Thus, Phascolarctobacterium can be used to prevent C. difficile infection (e.g., decrease the likelihood that a patient will become infected with C. difficile) and Phascolarctobacterium can be used to treat patients already infected with C. difficile. However, single-dose pre-treatment of Phascolarctobacterium was not sufficient to prevent C. difficile colonization and subsequent lethal colitis (FIG. 16), suggesting that Phascolarctobacterium must be present in an optimal number in the intestine to maximize the protective effect against CDI.

Phascolarctobacterium spp. are not the only bacterial species regulated by IL-22-mediated gut conditioning. During the development of embodiments of the technology provided herein, LEfSe analysis indicated that unclassified Proteobacteria were also underrepresented in IL-22-neutralized HMA mice. Thus, these not-yet-identified bacteria are regulated by IL-22 and play an important role in the protection against CDI in addition to Phascolarctobacterium spp. Moreover, in addition to succinate, several other luminal metabolites were differentially abundant between control and IL-22-neutralized HMA mice. For example, taurocholate and taurine were significantly reduced in IL-22-neutralized HMA mice. Taurocholate is known to promote the germination of C. difficile spores (9) and therefore reduction of taurocholate may reduce CDI. However, microbiota-derived taurine is known to control the microbial-host mutualism by modulating production of NLRP6-mediated anti-microbial peptides by the intestinal epithelial cells (46). Hence, reducing microbiota-derived taurine may also play a role in the increased susceptibility to CDI in IL-22-neutralized HMA mice Likewise, the concentration of various luminal amino acids was significantly increased as a result of gut dysbiosis. Since amino acid availability is closely associated with the risk for CDI (47), those may also contribute to the susceptible phenotype observed in those mice. Consistent with this notion, the Cd-CD2344-strain, which cannot utilize succinate, was still able to proliferate in IL-22-neutralized HMA mice in later infection, although the early colonization of this strain was dramatically impaired compared to the wild-type strain. This result indicates that succinate is an important metabolite that promotes the proliferation of C. difficile in the gut early after its colonization. Then, in later colonization, C. difficile may be able to use alternative nutritional sources (e.g., amino acids) for its growth. Thus, targeting different metabolic pathways, such as bile acid metabolism, succinate metabolism, and/or amino acid metabolism by utilizing multiple strains of commensal bacteria may provide an additional rational strategy to prevent CDI.

Accordingly, embodiments of the present technology relate to compositions comprising an organism of the genus Phascolarctobacterium, methods of using (e.g., administering) Phascolarctobacterium, kits comprising Phascolarctobacterium, systems comprising Phascolarctobacterium, and uses of Phascolarctobacterium, e.g., to provide a composition, treat a subject (e.g., for CDI), diagnose a subject, etc. In some embodiments, the technology relates to (e.g., comprises use of) a Phascolarctobacterium species that is, e.g., Phascolarctobacterium faecium (e.g., Phascolarctobacterium faecium 4_1_13G, Phascolarctobacterium faecium DSM 14760, Phascolarctobacterium faecium JCM30894, or Phascolarctobacterium faecium UC1_BHI_D), Phascolarctobacterium succinatutens (e.g., Phascolarctobacterium succinatutens CAG:287, Phascolarctobacterium succinatutens JCM16074, or Phascolarctobacterium succinatutens YIT 12067), or another Phascolarctobacterium sp. such as, e.g., Phascolarctobacterium sp. 377, Phascolarctobacterium sp. 3_1syn4, Phascolarctobacterium sp. CAG:207, Phascolarctobacterium sp. CAG:266, Phascolarctobacterium sp. canine oral taxon 149, Phascolarctobacterium sp. canine oral taxon 212, Phascolarctobacterium sp. ORNL_6EZ5-Gt_1_P12-107, Phascolarctobacterium sp. ORNL_6EZ5-Gt_3_Pl1-20, Phascolarctobacterium sp. ORNL_6EZ5-Gt_3_Pl1-21, Phascolarctobacterium sp. ORNL_6EZ5-Gt_3_Pl1-22, Phascolarctobacterium sp. ORNL_6EZ5-Gt_3_Pl1-23, Phascolarctobacterium sp. ORNL_6EZ5-Gt_3_Pl1-24, Phascolarctobacterium sp. ORNL_6EZ5-Gt_3_Pl1-25, Phascolarctobacterium sp. ORNL_6EZ5-Gt_3_Pl1-29, Phascolarctobacterium sp. ORNL_6EZ5-Gt_3_Pl2-123, Phascolarctobacterium sp. ORNL_6EZ5-Gt_3_Pl12-128, Phascolarctobacterium sp. ORNL_V42_A10, Phascolarctobacterium sp. ORNL_V42_C04, Phascolarctobacterium sp. ORNL_V42_C05, Phascolarctobacterium sp. ORNL_W42_B04, Phascolarctobacterium sp. ORNL_W42_B06, Phascolarctobacterium sp. ORNL_W42_B08, Phascolarctobacterium sp. ORNL_W42_B10, Phascolarctobacterium sp. ORNL_W42_B12, Phascolarctobacterium sp. ORNL_W42_C01, or Phascolarctobacterium sp. ORNL_W42_C09. In some embodiments, the Phascolarctobacterium sp. is an uncultured Phascolarctobacterium sp. In some embodiments, the technology relates to use of an organism that has a ribosomal RNA gene (e.g., 16S ribosomal RNA gene) sequence having approximately 97% identity to a ribosomal RNA gene (e.g., 16S ribosomal RNA gene) sequence from Phascolarctobacterium faecium JCM30894 and/or Phascolarctobacterium succinatutens JCM16074.

In some embodiments, the technology relates to use of gut commensal bacteria that degrade glycans and/or utilize glycans for growth. In some embodiments, the technology relates to use of gut commensal bacteria that degrade succinate and/or utilize succinate for growth. In some embodiments, the technology relates to gut commensal bacteria that decrease the amount of glycan and/or succinate in the gut.

### Probiotics, Pharmaceutical Compositions

As used herein, the term “probiotic bacterium” or “probiotic” refers to a live microorganism (e.g., Phascolarctobacterium sp.) or a composition comprising a live microorganism (e.g., Phascolarctobacterium sp.) that, when administered in adequate amounts, confers a health benefit on the subject to whom it is administered. In some embodiments, a probiotic comprises a microorganism that forms at least a part of the transient or endogenous microbiota (e.g., Phascolarctobacterium sp.) and thereby exhibits a beneficial prophylactic and/or therapeutic effect on a host organism. Probiotics are generally known to be clinically safe (e.g., non-pathogenic) by those individuals skilled in the art.

In some embodiments, a probiotic is formulated for oral administration (e.g., an oral probiotic (e.g., in a food, drink, capsule, and/or tablet). In some embodiments, a probiotic is formulated for administration by colonoscopy, enema, suppository, or orogastric tube. In some embodiments, a probiotic comprises 10⁴ to 10⁹ CFU (e.g., 10⁴, 10⁵, 10⁶, 10⁷, 10⁸, or 10⁹ CFU) of a probiotic microorganism (e.g., Phascolarctobactorium sp.). In some embodiments, a probiotic composition comprises saline, water, or milk. In some embodiments, compositions comprise a source of nutrients for the microorganisms in the probiotic composition.

In some embodiments, a composition according to the technology provided herein is administered to a subject and produces an increased amount of one or more Phascolarctobacterium spp. in the gut of the subject. In some embodiments, a composition is administered that results in approximately 3-10 log cells of Phascolarctobactorium spp. per gram of gut contents. In some embodiments, a composition is administered that results in more than 10 log cells of Phascolarctobactorium spp. per gram of gut contents. In some embodiments, a composition is provided to a subject that results in gut colonization of the gut by one or more Phascolarctobactorium spp. at a rate of approximately 3.22-5.76 log cells g⁻¹ gut contents for human infants (e.g., less than 1 year old), a rate of approximately 3.06-9.33 log cells g⁻¹ gut contents for humans aged 1 to 10, a rate of approximately 4.20-9.26 log cells g⁻¹ gut contents for humans aged 10 to 30 years old, a rate of approximately 3.86-9.03 log cells gut contents for humans aged 30 to 60 years old, and a rate of approximately 3.18-9.37 log cells g⁻¹ gut contents for humans older than 60 years old. In some embodiments, a composition is provided to a subject that results in gut colonization of the gut by one or more Phascolarctobactorium spp. at a rate of approximately 0.004 to 1.479 as a permillage of P. faecium in total gut bacteria.

It is generally contemplated that the compositions related to the technology are formulated for administration to a mammal, and especially to a human with a condition (e.g., CDI) that is responsive to the administration of such compounds or to prevent a condition or reduce the likelihood that the human will have a condition. Therefore, compositions are formulated in admixture with a pharmaceutically acceptable carrier. In some embodiments, compositions comprise an agent to stabilize pH, e.g., a buffer such as a phosphate, bicarbonate, or citrate buffer. Of course, one of ordinary skill in the art may modify the compositions within the teachings of the specification to provide a formulation for a particular route of administration.

Provided are compositions comprising bacteria (e.g., Phascolarctobacterium sp.) of the gut microbiota (e.g., mammalian gut microbiota (e.g., human gut microbiota)) with the capacity to provide functions of a healthy microbiota or promote an augmentation to the resident microbiome when administered to mammalian hosts. In particular, provided herein are compositions comprising Phascolarctobacterium sp. that treat, prevent, delay, or reduce the symptoms of diseases, disorders, and conditions associated with a dysbiosis (e.g., CDI). Without being limited to a specific mechanism, it is thought that the compositions described herein inhibit the growth, proliferation, and/or colonization of one or a plurality of pathogenic bacteria (e.g., C. difficile) in the dysbiotic microbiotal niche so that a healthy, diverse, and protective microbiota colonizes and populates the intestinal lumen to establish or reestablish ecological control over pathogens or potential pathogens (e.g., some bacteria are pathogenic bacteria only when present in a dysbiotic environment).

The bacterial compositions provided herein are produced and the efficacy thereof in inhibiting pathogenic bacteria is demonstrated as provided in further detail herein.

In some embodiments, compositions comprising bacteria (e.g., a Phascolarctobacterium sp.) may comprise a single species of bacteria, e.g., as determined by microbiological and/or bacteriological characterization of bacterial species and/or by molecular genetic technologies such as nucleic acid sequencing, sequence comparison, and phylogenetics. In some embodiments, compositions comprise a single OTU of bacteria, e.g., the composition comprises bacteria having greater than 97% (e.g., at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100.0%) identity in a nucleic acid sequence (e.g., a ribosomal RNA (e.g., a 16S rRNA) sequence). For example, in some embodiments, compositions comprise a single species from the genus Phascolarctobacterium such as P. faecium JCM30894 or P. succinatutens JCM16074. In some embodiments, compositions comprise two types of bacteria or greater than two types of bacteria (e.g., from the genus Phascolarctobacterium such as P. faecium JCM30894 or P. succinatutens JCM16074 or other Phascolarctobacterium species described herein and/or known in the art). For instance, in some embodiments compositions comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21, 22, 23, 24, 25, 26, 27, 28, 29 30, 31, 32, 33, 34, 35, 36, 37, 38, 39, or at least 40, at least 50, or greater than 50 types of bacteria, as defined by species or operational taxonomic unit (OTU), or otherwise as provided herein.

In some embodiments, the number of types of bacteria present in a composition is at or below a known value. For example, in some embodiments the bacterial composition comprises 50 or fewer types of bacteria, such as 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 or fewer, or 9 or fewer types of bacteria, 8 or fewer types of bacteria, 7 or fewer types of bacteria, 6 or fewer types of bacteria, 5 or fewer types of bacteria, 4 or fewer types of bacteria, or 3 or fewer types of bacteria. In another embodiment, a bacterial composition comprises from 2 to no more than 40, from 2 to no more than 30, from 2 to no more than 20, from 2 to no more than 15, from 2 to no more than 10, or from 2 to no more than 5 types of bacteria. In some embodiments, at least one of the types of bacteria is from the genus Phascolarctobacterium (e.g., a species such as P. faecium JCM30894 or P. succinatutens JCM16074).

In some embodiments, compositions are provided with the ability to exclude, inhibit growth of, and/or minimize the amount of pathogenic bacteria. Exemplary bacterial compositions are demonstrated to reduce the growth rate and/or amount of C. difficile in the gut as provided in the Examples.

In some embodiments, compositions with the capacity to exclude C. difficile are developed using a methodology for estimating an Ecological Control Factor (ECF) for constituents within the human microbiota. The ECF is determined by assessing the antagonistic activity of a given commensal strain or combination of strains towards a given pathogen using an in vitro assay, resulting in observed levels of ecological control at various concentrations of the added commensal strains. The ECF for a commensal strain or combination of strains is somewhat analogous to the conventional minimal inhibitory concentration (MIC) assessment that is employed in the assessment of antibiotics. The ECF allows for the assessment and ranking of relative potencies of commensal strains, combinations of strains, and compositions comprising bacteria for their ability to antagonize gastrointestinal pathogens. The ECF of a commensal strain or combination of strains may be calculated by assessing the concentration of that composition that is able to mediate a given percentage of inhibition (e.g., at least 10%, 20%, 50%, 70%, 75%, 80%, 85%, 90%, 95%, or 100%) of a target pathogen in the in vitro assay. Provided herein are compositions comprising bacteria, strains, and/or combinations of bacteria or strains or OTUs within the human microbiota that are able to significantly reduce the rate of gastrointestinal pathogen (e.g., C. difficile) replication within the in vitro assay. These compositions are capable of providing a safe and effective means by which to affect the growth, replication, and disease severity of such bacterial pathogens.

In some embodiments, an in vivo mouse model is used to test for the protective effect of composition described herein against C. difficile. In some embodiments, (e.g., based on Chen (2008) “A mouse model of Clostridium difficile associated disease” Gastroenterology 135: 1984-92, incorporated herein by reference), mice are made susceptible to C. difficile by a 7-day treatment (days −12 to −5 of experiment) with 5 to 7 antibiotics (e.g., kanamycin, colistin, gentamycin, metronidazole, and vancomycin and optionally including ampicillin and ciprofloxacin) delivered by drinking water, followed by a single dose with Clindamycin on day −3, then challenged three days later on day 0 with 10⁴ spores of C. difficile via oral gavage (e.g., oro-gastric lavage). Bacterial compositions may be given either before (prophylactic treatment) or after (therapeutic treatment) C. difficile gavage. Further, bacterial compositions may be given after (optional) vancomycin treatment (see below) to assess their ability to prevent recurrence and thus suppress the pathogen in vivo. The outcomes assessed each day from day—1 to day 6 (or beyond, for prevention of recurrence) are weight, clinical signs, mortality, and shedding of C. difficile in the feces. Weight loss, clinical signs of disease, and C. difficile shedding are typically observed without treatment. Vancomycin provided by oral gavage on days—1 to 4 protects against these outcomes and serves as a positive control. Clinical signs are subjective and scored each day by an experienced observer. In some embodiments, animals that lose greater than or equal to 25% of their body weight are euthanized and counted as infection-related mortalities. Feces are gathered from mouse cages and the shedding of C. difficile spores is detected in the feces using a selective plating assay or via qPCR. Alternative dosing schedules and routes of administration (e.g. rectal) may be employed, including multiple doses and 10³ to 10¹⁰ of a given organism or composition may be delivered.

In some embodiments, methods for producing bacterial compositions include, e.g., processing steps combined with one or more mixing steps. In some embodiments, steps include one or more of organism banking, organism production, and preservation.

For example, in some embodiments, bacteria (e.g., species or strains) are banked, e.g., one or more species included in a bacterial composition are (1) isolated directly from a specimen or taken from a banked stock, (2) optionally cultured on a nutrient agar or broth that supports growth to generate viable biomass, and (3) the biomass optionally preserved in multiple aliquots in long-term storage. In some embodiments, bacteria (e.g., species or strains) are cultured. For example, in some embodiments, bacteria are grown in an agar or broth comprising nutrients that provide essential elements and specific factors that enable growth. A variety of microbiological media and variations are well known in the art (see, e.g., R. M. Atlas, Handbook of Microbiological Media (2010) CRC Press, incorporated herein by reference). Medium can be added to the culture at the start, may be added during the culture, or may be intermittently/continuously flowed through the culture. The bacteria (e.g., species and/or strains) in the bacterial composition may be cultivated alone, as a subset of the bacterial composition, or as an entire collection comprising the bacterial composition. As an example, a first strain may be cultivated together with a second strain in a mixed continuous culture, at a dilution rate lower than the maximum growth rate of either cell to prevent the culture from washing out of the cultivation.

The inoculated culture is incubated under favorable conditions for a time sufficient to build biomass. In some embodiments, bacterial compositions for human use are often grown at 37° C., at a pH, and under other growth conditions similar to the normal human niche (e.g., the gut). The environment may be actively controlled, passively controlled (e.g., via buffers), or allowed to drift. For example, for anaerobic bacterial compositions (e.g., gut microbiota), an anoxic/reducing environment may be employed. This can be accomplished by adding reducing agents such as cysteine to the broth, and/or stripping it of oxygen.

When the culture has generated sufficient biomass, it may be preserved for banking. The organisms may be placed into a composition comprising components that protect bacteria from freezing (cryoprotectants), drying (lyoprotectants), and/or osmotic shock (osmoprotectants), dispensing into containers, and treating the culture for preservation. Containers are generally impermeable and have closures that assure isolation from the environment. Cryopreservation treatment is accomplished by freezing a liquid at ultra-low temperatures (e.g., at or below −80° C.). Dried preservation removes water from the culture by evaporation (in the case of spray drying or cool drying) or by sublimation (e.g., for freeze drying and/or spray freeze drying). Removal of water improves long-term bacterial composition storage stability at temperatures elevated above cryogenic. If the bacterial composition comprises spore forming species and results in the production of spores, the final composition may be purified by additional means such as density gradient centrifugation preserved using the techniques described above. Bacterial composition banking may be done by culturing and preserving the strains individually, or by mixing the strains together to create a combined bank. As an example of cryopreservation, a bacterial composition culture may be harvested by centrifugation to pellet the cells from the culture medium, the supernatant decanted and replaced with fresh culture broth containing 15% glycerol. The culture can then be aliquoted into 1-mL cryotubes, sealed, and placed at −80° C. for long-term viability retention. This procedure achieves acceptable viability upon recovery from frozen storage.

Production of organisms for use according to embodiments of the therapeutic technologies provided herein are conducted using similar culture steps to banking, including use of similar growth medium and culture conditions. In some embodiments, production is conducted at larger scales of operation, especially for clinical development or commercial production. At larger scales, several subcultivations of the bacterial composition may be used prior to the final cultivation. At the end of cultivation, the culture is harvested to enable further formulation into a dosage form for administration. Formulation can involve concentration, removal of undesirable medium components, and/or introduction into a chemical milieu that preserves the bacterial composition and renders it acceptable for administration via the chosen route. For example, a bacterial composition may be cultivated to a specified concentration of between 10⁶ to 10¹⁰ CFU/mL, then concentrated (e.g., by tangential flow microfiltration). Spent medium may be exchanged by diafiltering with a preservative medium. The suspension can then be freeze-dried. After drying, the powder may be blended to an appropriate potency, and mixed with other cultures and/or a filler such as microcrystalline cellulose for consistency and ease of handling, and the bacterial composition formulated as provided herein.

Embodiments of the technology relate to formulations for administration to humans and other subjects in need thereof. In some embodiments, the bacterial compositions are combined with additional active and/or inactive materials to produce a final product, which may be in single dosage unit or in a multi-dose format. In some embodiments, the composition comprises at least one carbohydrate. A “carbohydrate” refers to a sugar or polymer of sugars. The terms “saccharide”, “polysaccharide”, “carbohydrate”, and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

In some embodiments, compositions comprise at least one lipid. As used herein, a “lipid” includes fats, oils, triglycerides, cholesterol, phospholipids, and/or fatty acids in any form including free fatty acids. Fats, oils, and fatty acids can be saturated, unsaturated (cis or trans), or partially unsaturated (cis or trans). In some embodiments, the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some embodiments the composition comprises at least one modified lipid, for example a lipid that has been modified by cooking.

In some embodiments, compositions comprise at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, compositions comprise at least one supplemental vitamin. The at least one vitamin can be a fat-soluble or water-soluble vitamin. Suitable vitamins include but are not limited to vitamin C, vitamin A, vitamin E, vitamin B₁₂, vitamin K, riboflavin, niacin, vitamin D, vitamin B₆, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, compositions comprise an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent. In some embodiments, the excipient is a buffering agent. Non-limiting examples of suitable buffering agents include sodium citrate, magnesium carbonate, magnesium bicarbonate, calcium carbonate, and calcium bicarbonate. In some embodiments, the excipient comprises a preservative. Non-limiting examples of suitable preservatives include antioxidants, such as alpha-tocopherol and ascorbate, and antimicrobials, such as parabens, chlorobutanol, and phenol. In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, compositions comprise a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, compositions comprise a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, compositions comprise a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments the excipient comprises a flavoring agent. Flavoring agents can be chosen from synthetic flavor oils and flavoring aromatics; natural oils; extracts from plants, leaves, flowers, and fruits; and combinations thereof. In some embodiments the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, compositions comprise a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

In some embodiments, the weight fraction of the excipient or combination of excipients in the formulation is usually approximately 99% or less, such as approximately 95% or less, approximately 90% or less, approximately 85% or less, approximately 80% or less, approximately 75% or less, approximately 70% or less, approximately 65% or less, approximately 60% or less, approximately 55% or less, 50% or less, approximately 45% or less, approximately 40% or less, approximately 35% or less, approximately 30% or less, approximately 25% or less, approximately 20% or less, approximately 15% or less, approximately 10% or less, approximately 5% or less, approximately 2% or less, or approximately 1% or less of the total weight of the composition.

The bacterial compositions disclosed herein can be formulated into a variety of forms and administered by a number of different means. The compositions can be administered orally, rectally, enterically, or parenterally, in formulations containing conventionally acceptable carriers, adjuvants, and vehicles as desired. The term “parenteral” as used herein includes subcutaneous, intravenous, intramuscular, or intrasternal injection and infusion techniques. In some embodiments, the bacterial composition is administered orally. In some embodiments, the bacterial composition is administered rectally.

Solid dosage forms for oral administration include capsules, tablets, caplets, pills, troches, lozenges, powders, and granules. A capsule typically comprises a core material comprising a bacterial composition and a shell wall that encapsulates the core material. In some embodiments the core material comprises at least one of a solid, a liquid, and an emulsion. In some embodiments the shell wall material comprises at least one of a soft gelatin, a hard gelatin, and a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer functions as taste-masking agents.

Tablets, pills, and the like can be compressed, multiply compressed, multiply layered, and/or coated. The coating can be single or multiple. In one embodiment, the coating material comprises at least one of a saccharide, a polysaccharide, and glycoproteins extracted from at least one of a plant, a fungus, and a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments the coating material comprises a protein. In some embodiments the coating material comprises at least one of a fat and an oil. In some embodiments the at least one of a fat and an oil is high temperature melting. In some embodiments the at least one of a fat and an oil is hydrogenated or partially hydrogenated. In some embodiments the at least one of a fat and an oil is derived from a plant. In some embodiments the at least one of a fat and an oil comprises at least one of glycerides, free fatty acids, and fatty acid esters. In some embodiments the coating material comprises at least one edible wax. The edible wax can be derived from animals, insects, or plants. Non-limiting examples include beeswax, lanolin, bayberry wax, carnauba wax, and rice bran wax. Tablets and pills can additionally be prepared with enteric coatings.

Alternatively, powders or granules embodying the bacterial compositions disclosed herein can be incorporated into a food product. In some embodiments the food product is a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In some embodiments, the compositions disclosed herein are incorporated into a therapeutic food. In some embodiments, the therapeutic food is a ready-to-use food that optionally contains some or all essential macronutrients and micronutrients. In some embodiments, the compositions disclosed herein are incorporated into a supplementary food that is designed to be blended into an existing meal. In some embodiments, the supplemental food contains some or all essential macronutrients and micronutrients. In some embodiments, the bacterial compositions disclosed herein are blended with or added to an existing food to fortify the food's protein nutrition. Examples include food staples (grain, salt, sugar, cooking oil, margarine), beverages (coffee, tea, soda, beer, liquor, sports drinks), snacks, sweets and other foods. In some embodiments, a bacterial composition as described herein is provided as a food product, e.g., a solid foodstuff. Suitable examples of a solid foodstuff include without limitation a food bar, a snack bar, a cookie, a brownie, a muffin, a cracker, an ice cream bar, a frozen yogurt bar, and the like.

In some embodiments, the formulations are filled into gelatin capsules for oral administration. An example of an appropriate capsule is a 250-mg gelatin capsule containing from 10 (up to 100 mg) of lyophilized powder (10⁸ to 10¹¹ bacteria), approximately 100-200 mg microcrystalline cellulose, approximately 50-100 mg gelatin, and approximately 1-5 mg magnesium stearate. In an alternative embodiment, from 10⁵ to 10¹² bacteria may be used, 10⁵ to 10⁷, 10⁶ to 10⁷, or 10⁸ to 10¹⁰, with attendant adjustments of the excipients if necessary. In an alternative embodiment an enteric-coated capsule or tablet or with a buffering or protective composition may be used.

In some embodiments comprising more than one type (e.g., species, strain, or OTU) of bacteria, the number of bacteria of each type may be present in the same amount or in different amounts. For example, in a bacterial composition with two types of bacteria, the bacteria may be present in from a 1:10,000 ratio to a 1:1 ratio, from a 1:10,000 ratio to a 1:1,000 ratio, from a 1:1,000 ratio to a 1:100 ratio, from a 1:100 ratio to a 1:50 ratio, from a 1:50 ratio to a 1:20 ratio, from a 1:20 ratio to a 1:10 ratio, from a 1:10 ratio to a 1:1 ratio. For bacterial compositions comprising at least three types of bacteria, the ratio of type of bacteria may be chosen pairwise from ratios for bacterial compositions with two types of bacteria. For example, in a bacterial composition comprising bacteria A, B, and C, at least one of the ratios between bacteria A and B, the ratio between bacteria B and C, and the ratio between bacteria A and C may be chosen, independently, from the pairwise combinations above.

In some embodiments the compositions disclosed herein are administered to a patient or a user (sometimes collectively referred to as a “subject”). As used herein, “administer” and “administration” encompasses embodiments in which one person directs another to consume a bacterial composition in a certain manner and/or for a certain purpose and situations in which a user uses a bacteria composition in a certain manner and/or for a certain purpose independently of or in variance to any instructions received from a second person. Non-limiting examples of embodiments in which one person directs another to consume a bacterial composition in a certain manner and/or for a certain purpose include when a physician prescribes a course of conduct and/or treatment to a patient, when a parent commands a minor user (such as a child) to consume a bacterial composition, when a trainer advises a user (such as an athlete) to follow a particular course of conduct and/or treatment, and when a manufacturer, distributer, or marketer recommends conditions of use to an end user, for example through advertisements, inserts, or labeling on packaging or on other materials provided in association with the sale or marketing of a product.

The bacterial compositions offer a protective and/or therapeutic effect against infection by one or more GI pathogens of interest (e.g., C. difficile) and thus may be administered after an acute case of infection has been resolved in order to prevent relapse, during an acute case of infection (e.g., in some embodiments, as a complement to antibiotic therapy), or to prevent infection or reduce transmission from disease carriers.

The present bacterial compositions may be useful in a variety of clinical situations. For example, the bacterial compositions may be administered as a complementary treatment (e.g., complementary to antibiotic treatments) when a patient is suffering from an acute infection, to reduce the risk of recurrence after an acute infection has subsided, or when a patient will be in close proximity to others with or at risk of serious gastrointestinal infections (physicians, nurses, hospital workers, family members of those who are ill or hospitalized).

The present bacterial compositions may be administered to animals, including humans, laboratory animals (e.g., primates, rats, mice), livestock (e.g., cows, sheep, goats, pigs, turkeys, chickens), and household pets (e.g., dogs, cats, rodents).

In some embodiments, the bacterial composition is administered enterically (e.g., by a route of access to the gastrointestinal tract). This includes oral administration, rectal administration (including enema, suppository, or colonoscopy), by an oral or nasal tube (nasogastric, nasojejunal, oral gastric, or oral jejunal), as detailed more fully herein.

Prior to administration of the bacterial composition, the patient may optionally have a pretreatment protocol to prepare the gastrointestinal tract to receive the bacterial composition. In certain embodiments, the pretreatment protocol is advisable, such as when a patient has an acute infection with a highly resilient pathogen. In other embodiments, the pretreatment protocol is entirely optional, such as when the pathogen causing the infection is not resilient, or the patient has had an acute infection that has been successfully treated but where the physician is concerned that the infection may recur. In these instances, the pretreatment protocol may enhance the ability of the bacterial composition to affect the patient's microbiome.

As one way of preparing the patient for administration of a compositions comprising bacteria as described herein (e.g., bacteria from the genus Phascolarctobacterium such as P. faecium JCM30894 or P. succinatutens JCM16074), at least one antibiotic may be administered to alter the bacteria in the patient. As another way of preparing the patient for administration of the microbial ecosystem, a standard colon-cleansing preparation may be administered to the patient to substantially empty the contents of the colon, such as used to prepare a patient for a colonscopy. As used herein, the term “substantially emptying the contents of the colon” refers to removing at least 75%, at least 80%, at least 90%, at least 95%, or approximately 100% of the contents of the ordinary volume of colon contents. Antibiotic treatment may precede the colon-cleansing protocol.

If a patient has received an antibiotic for treatment of an infection or if a patient has received an antibiotic as part of a specific pretreatment protocol, in one embodiment, the antibiotic should be stopped in sufficient time to allow the antibiotic to be substantially reduced in concentration in the gut before the bacterial composition is administered. In one embodiment, the antibiotic may be discontinued 1, 2, or 3 days before the administration of the bacterial composition. In one embodiment, the antibiotic may be discontinued 3, 4, 5, 6, or 7 antibiotic half-lives before administration of the bacterial composition. In another embodiment, the antibiotic may be chosen so the constituents in the bacterial composition have an MIC50 that is higher than the concentration of the antibiotic in the gut.

MIC50 of a bacterial composition or the elements in the composition may be determined by methods well known in the art. See, e.g., Reller et al., (2009) “Antimicrobial Susceptibility Testing: A Review of General Principles and Contemporary Practices” Clinical Infectious Diseases 49: 1749-55, incorporated herein by reference. In some embodiments, the additional time between antibiotic administration and administration of the bacterial composition is not necessary. If the pretreatment protocol is part of treatment of an acute infection, the antibiotic may be chosen so that the infection is sensitive to the antibiotic, but the constituents in the bacterial composition are not sensitive to the antibiotic.

The bacterial compositions of the invention are suitable for administration to mammals and non-mammalian animals in need thereof. In certain embodiments, the mammalian subject is a human subject who has one or more symptoms of a dysbiosis (e.g., a CDI and/or has an increased likelihood of having a CDI).

When the mammalian subject is suffering from a disease, disorder, or condition characterized by an aberrant microbiota, the bacterial compositions described herein are suitable for treatment thereof. In some embodiments, the mammalian subject has not received antibiotics in advance of treatment with the bacterial compositions. For example, the mammalian subject has not been administered at least two doses of vancomycin, metronidazole, and/or or similar antibiotic compound within one week prior to administration of the therapeutic composition. In other embodiments, the mammalian subject has not previously received an antibiotic compound in the one month prior to administration of the therapeutic composition. In other embodiments, the mammalian subject has received one or more treatments with one or more different antibiotic compounds and such treatment(s) resulted in no improvement or a worsening of symptoms.

In some embodiments, the gastrointestinal disease, disorder or condition is diarrhea caused by C. difficile, including recurrent C. difficile infection, ulcerative colitis, colitis, Crohn's disease, or irritable bowel disease. Beneficially, the therapeutic composition is administered only once prior to improvement of the disease, disorder or condition. In some embodiments, the therapeutic composition is administered at intervals greater than two days, such as once every three, four, five, or six days, or every week or less frequently than every week. In some embodiments, the preparation is administered intermittently according to a set schedule, e.g., once a day, once weekly, or once monthly, or when the subject relapses from the primary illness. In another embodiment, the preparation may be administered on a long-term basis to subjects who are at risk for infection with or who may be carriers of these pathogens, including subjects who will have an invasive medical procedure (such as surgery), who will be hospitalized, who live in a long-term care or rehabilitation facility, who are exposed to pathogens by virtue of their profession (livestock and animal processing workers), or who could be carriers of pathogens (including hospital workers such as physicians, nurses, and other health care professionals).

In some embodiments, the bacterial composition is administered enterically. This preferentially includes oral administration, or by an oral or nasal tube (including nasogastric, nasojejunal, oral gastric, or oral jejunal). In other embodiments, administration includes rectal administration (including enema, suppository, or colonoscopy). The bacterial composition may be administered to at least one region of the gastrointestinal tract, including the mouth, esophagus, stomach, small intestine, large intestine, and rectum. In some embodiments, the composition is administered to all regions of the gastrointestinal tract. The bacterial compositions may be administered orally in the form of medicaments such as powders, capsules, tablets, gels, or liquids. The bacterial compositions may also be administered in gel or liquid form by the oral route or through a nasogastric tube, or by the rectal route in a gel or liquid form, by enema or instillation through a colonoscope or by a suppository.

If the composition is administered colonoscopically and, optionally, if the bacterial composition is administered by other rectal routes (such as an enema or suppository) or even if the subject has an oral administration, the subject may have a colon cleansing preparation. The colon-cleansing preparation can facilitate proper use of the colonoscope or other administration devices, but even when it does not serve a mechanical purpose it can also maximize the proportion of the bacterial composition relative to the other organisms previously residing in the gastrointestinal tract of the subject. Any ordinarily acceptable colon-cleansing preparation may be used such as those typically provided when a subject undergoes a colonoscopy.

In some embodiments, the bacteria and bacterial compositions are provided in a dosage form. In some embodiments the dosage form is designed for administration of at least one OTU or combination thereof disclosed herein, wherein the total amount of bacterial composition administered is selected from 0.1 ng to 10 g, 10 ng to 1 g, 100 ng to 0.1 g, 0.1 mg to 500 mg, 1 mg to 100 mg, or from 10-15 mg. In some embodiments, the bacterial composition is consumed at a rate of from 0.1 ng to 10 g a day, 10 ng to 1 g a day, 100 ng to 0.1 g a day, 0.1 mg to 500 mg a day, 1 mg to 100 mg a day, or from 10-15 mg a day, or more.

In some embodiments, the treatment period is at least 1 day, at least 2 days, at least 3 days, at least 4 days, at least 5 days, at least 6 days, at least 1 week, at least 2 weeks, at least 3 weeks, at least 4 weeks, at least 1 month, at least 2 months, at least 3 months, at least 4 months, at least 5 months, at least 6 months, or at least 1 year. In some embodiments, the treatment period is from 1 day to 1 week, from 1 week to 4 weeks, from 1 month, to 3 months, from 3 months to 6 months, from 6 months to 1 year, or for over a year.

In some embodiments, from 10⁵ and 10¹² microorganisms are administered to the patient in a given dosage form. In one mode, an effective amount may be provided in from 1 to 500 ml or from 1 to 500 grams of the bacterial composition having from 10⁷ to 10¹¹ bacteria per ml or per gram, or a capsule, tablet, or suppository having from 1 mg to 1000 mg lyophilized powder having from 10⁷ to 10¹¹ bacteria. Those receiving acute treatment may receive higher doses than those who are receiving chronic administration (such as hospital workers or those admitted into long-term care facilities).

Any of the preparations described herein may be administered once on a single occasion or on multiple occasions, such as once a day for several days or more than once a day on the day of administration (including twice daily, three times daily, or up to five times daily). In some embodiments, the preparation may be administered intermittently according to a set schedule, e.g., once weekly, once monthly, or when the patient relapses from the primary illness. In some embodiments, the preparation may be administered on a long-term basis to individuals who are at risk for infection with or who may be carriers of these pathogens, including individuals who will have an invasive medical procedure (such as surgery), who will be hospitalized, who live in a long-term care or rehabilitation facility, who are exposed to pathogens by virtue of their profession (livestock and animal processing workers), or who could be carriers of pathogens (including hospital workers such as physicians, nurses, and other health care professionals).

The bacterial compositions may be administered with other agents in a combination therapy mode, including anti-microbial agents and prebiotics. Administration may be sequential, over a period of hours or days, or simultaneous. In some embodiments, bacterial compositions are administered with IL-22. In some embodiments, bacterial compositions are administered with an agent (e.g., a drug, a bacterium, etc.) that increases IL-22 production in the gut.

In one embodiment, the bacterial compositions are included in combination therapy with one or more anti-microbial agents, which include anti-bacterial agents, anti-fungal agents, anti-viral agents and anti-parasitic agents. Anti-bacterial agents include cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); and carbapenem antibiotics (ertapenem, doripenem, imipenem/cilastatin, and meropenem). Anti-viral agents include Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, Foscarnet, Fomivirsen, Ganciclovir, Indinavir, Idoxuridine, Lamivudine, Lopinavir Maraviroc, MK-2048, Nelfinavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir and Zidovudine. Examples of antifungal compounds include, but are not limited to polyene antifungals such as natamycin, rimocidin, filipin, nystatin, amphotericin B, candicin, and hamycin; imidazole antifungals such as miconazole, ketoconazole, clotrimazole, econazole, omoconazole, bifonazole, butoconazole, fenticonazole, isoconazole, oxiconazole, sertaconazole, sulconazole, and tioconazole; triazole antifungals such as fluconazole, itraconazole, isavuconazole, ravuconazole, posaconazole, voriconazole, terconazole, and albaconazole; thiazole antifungals such as abafungin; allylamine antifungals such as terbinafine, naftifine, and butenafine; and echinocandin antifungals such as anidulafungin, caspofungin, and micafungin. Other compounds that have antifungal properties include, but are not limited to polygodial, benzoic acid, ciclopirox, tolnaftate, undecylenic acid, flucytosine or 5-fluorocytosine, griseofulvin, and haloprogin.

In one embodiment, the bacterial compositions are included in combination therapy with one or more corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines, and combinations thereof.

In some embodiments, bacterial compositions are included in combination therapy with a cytokine, e.g., an interleukin such as, e.g., IL-22. In some embodiments, compositions comprise a bacterium (e.g., an organism from the genus Phascolarctobacterium such as P. faecium JCM30894 or P. succinatutens JCM16074) and IL-22. In some embodiments, compositions comprise a bacterium (e.g., an organism from the genus Phascolarctobacterium such as P. faecium JCM30894 or P. succinatutens JCM16074) and an agent and/or a second organism (e.g., bacterium) that induces IL-22 production in the gut. For example, some embodiments provide a composition comprising an organism from the genus Phascolarctobacterium such as P. faecium JCM30894 or P. succinatutens JCM16074 and a Lactobacillus. Lactobacilli species metabolize tryptophan to indole-3-acetic acid, which activates Ahr, and which, in turn, stimulates IL-22 production (see, e.g., Zelante (2013) “Tryptophan catabolites from microbiota engage aryl hydrocarbon receptor and balance mucosal reactivity via interleukin-22” Immunity 39: 372-85, incorporated herein by reference). In some embodiments, compositions comprise an organism from the genus Phascolarctobacterium such as P. faecium JCM30894 or P. succinatutens JCM16074 and tryptophan and/or indole-3-acetic acid. Tryptophan has been demonstrated to activate production of IL-22 (see, e.g., Zelante, supra and Lamas (2016) “CARD9 impacts colitis by altering gut microbiota metabolism of tryptophan into aryl hydrocarbon receptor ligands” Nat Med. 22: 598-605, each of which is incorporated herein by reference). In some embodiments, compositions comprise an organism from the genus Phascolarctobacterium such as P. faecium JCM30894 or P. succinatutens JCM16074 and an agent and/or second organism that induces Ahr activity, e.g., the ligands Ficz (see, e.g., Monteleone (2011) “Aryl hydrocarbon receptor-induced signals up-regulate IL-22 production and inhibit inflammation in the gastrointestinal tract” Gastroenterology 141: 237-48, incorporated herein by reference) and/or ABX464 (see, e.g., Chebli (2017) “The anti-HIV candidate Abx464 dampens intestinal inflammation by triggering 11-22 production in activated macrophages” Sci Rep. 7: 4860, incorporated herein by reference). Aryl hydrocarbon receptor (Ahr) is activated by a large variety of environmental toxins and induces IL-22 production (see, e.g., Monteleone (2013) “Aryl hydrocarbon receptor and colitis” Semin Immunopathol. 35: 671-75, incorporated herein by reference). Increasing the amount and/or activity of IL-22 and/or Ahr both have beneficial effects on the gut (see, e.g., Monteleone (2011) “Aryl hydrocarbon receptor - induced signals up-regulate IL-22 production and inhibit inflammation in the gastrointestinal tract” Gastroenterology 141: 237-48, incorporated herein by reference). In some embodiments, compositions comprise an organism from the genus Phascolarctobacterium such as P. faecium JCM30894 or P. succinatutens JCM16074 and Xilei-San or a component isolated from Xilei-San. Xilei-San is a Chinese herbal medicine that has been used to treat gut ailments and that comprises a high concentration of indigo naturalis, a ligand of Ahr. Indigo naturalis produces Ahr-mediated activation of the IL-22 pathway (see, e.g., Kawai 92017) “Indigo naturalis ameliorates murine dextran sodium sulfate-induced colitis via aryl hydrocarbon receptor activation” J Gastroenterol. 52: 904-19, incorporated herein by reference). In some embodiments, compositions comprise an organism from the genus Phascolarctobacterium such as P. faecium JCM30894 or P. succinatutens JCM16074 and indigo naturalis.

In some embodiments, the technology comprises use of a composition comprising a bacterium (e.g., an organism from the genus Phascolarctobacterium such as P. faecium JCM30894 or P. succinatutens JCM16074) and use of an agent that minimizes and/or eliminates IL-22 binding protein (IL-22BP) or that minimizes and/or eliminates IL-22BP activity.

In some embodiments, compositions comprise a bacterium (e.g., an organism from the genus Phascolarctobacterium such as P. faecium JCM30894 or P. succinatutens JCM16074) and a recombinant IL-22 molecule (e.g., comprising one or more modification to increase in vivo stability). See, e.g., Stefanich (2018) “Pre-clinical and translational pharmacology of a human interleukin-22 IgG fusion protein for potential treatment of infectious or inflammatory diseases” Biochemical Pharmacology 152: 224-35; and Tang (2018) “Safety, pharmacokinetics, and biomarkers of F-652, a recombinant human interleukin-22 dimer, in healthy subjects” Cell Mol Immunol 16: 473-82, each of which is incorporated herein by reference. For example, in some embodiments, compositions comprise a recombinant IL-22-Fc molecule (UTR1147A) that has been tested in phase 1 trials (see, e.g., Lekkerkerker (2017) “P418 Pharmacodynamic biomarkers demonstrate dose-dependent pharmacological activity of the IL-22Fc fusion protein UTR1147A in healthy volunteers in a phase 1a clinical trial” Journal of Crohn's and Colitis, Volume 11, Issue suppl_1, 1 Feb. 2017, Page S289, incorporated herein by reference).

In some embodiments, the technology provides compositions comprising a bacterium (e.g., an organism from the genus Phascolarctobacterium such as P. faecium JCM30894 or P. succinatutens JCM16074) and a genetically modified organism (e.g., a bacterium) that expresses IL-22. For example, in some embodiments, the technology provides compositions comprising a bacterium (e.g., an organism from the genus Phascolarctobacterium such as P. faecium JCM30894 or P. succinatutens JCM16074) and a genetically modified Lactobacillus that expresses IL-22 (see, e.g., Lin (2017) “Lactobacillus delivery of bioactive interleukin-22” Microbial Cell Factories volume 16, Article number: 148, incorporated herein by reference). The technology is not limited to the use of Lactobacillus and includes expression of IL-22 by microorganisms that are genetically tractable and that are compatible with the human gut, including but not limited to Bacteroides, Bifidobacteria, Escherichia, and Lactobacillus. Further, the Human Microbiome Project provides several isolated bacterial strains from human fecal samples that are available through commercial stock centers and that have been sequenced. See “The NIH Human Microbiome Project” (2009) Genome Res. 19: 2317-23, incorporated herein by reference.

In some embodiments, the technology comprises use of a prebiotic. A prebiotic is a selectively fermented ingredient that allows specific changes, both in the composition and/or activity, in the gastrointestinal microbiota that confers benefits upon host well-being and health. Prebiotics may include complex carbohydrates, amino acids, peptides, or other essential nutritional components for the survival of the bacterial composition. Prebiotics include, but are not limited to, amino acids, biotin, fructo-oligosaccharide, galacto-oligosaccharides, inulin, lactulose, mannan oligosaccharides, oligofructose-enriched inulin, oligofructose, oligodextrose, tagatose, trans-galacto-oligosaccharide, and xylooligosaccharides.

In some embodiments, methods are provided for testing certain characteristics of bacterial compositions. For example, the sensitivity of bacterial compositions to certain environmental variables is determined, e.g., to select for particular desirable characteristics in a given composition, formulation, and/or use. For example, the constituents in the bacterial composition may be tested for pH resistance, bile acid resistance, and/or antibiotic sensitivity, either individually on a constituent-by-constituent basis or collectively as a bacterial composition comprising multiple bacterial constituents (collectively referred to as a bacterial composition).

In some embodiments, a composition is tested for sensitivity to pH. If a bacterial composition will be administered other than to the colon or rectum (for example, through, but not limited to, an oral route), optionally testing for pH resistance enhances the selection of bacterial compositions that will survive at the highest yield possible through the varying pH environments of the distinct regions of the GI tract. Understanding how the bacterial compositions react to the pH of the GI tract also assists in formulation, so that the number of bacteria in a dosage form can be increased if beneficial and/or so that the composition may be administered in an enteric-coated capsule or tablet or with a buffering or protective composition. As the pH of the stomach can drop to a pH of 1 to 2 after a high-protein meal for a short time before physiological mechanisms adjust it to a pH of 3 to 4 and often resides at a resting pH of 4 to 5, and as the pH of the small intestine can range from a pH of 6 to 7.4, bacterial compositions can be prepared that survives these varying pH ranges (specifically wherein at least 1%, 5%, 10%, 15%, 20%, 25%, 30%, 40%, 50%, 60%, 70%, 80%, 90%, or as much as 100% of the bacteria can survive gut transit times through various pH ranges). This may be tested by exposing the bacterial composition to varying pH ranges for the expected gut transit times through those pH ranges. Therefore, as a nonlimiting example only, 18-hour cultures of bacterial compositions may be grown in standard media, such as gut microbiota medium (“GMM”, see Goodman et al., (2011) “Extensive personal human gut microbiota culture collections characterized and manipulated in gnotobiotic mice” PNAS 108: 6252-57, incorporated herein by reference) or another animal-products-free medium, with the addition of pH adjusting agents for a pH of 1 to 2 for 30 minutes, a pH of 3 to 4 for 1 hour, a pH of 4 to 5 for 1 to 2 hours, and a pH of 6 to 7.4 for 2.5 to 3 hours. An alternative method for testing stability to acid is described in U.S. Pat. No. 4,839,281, incorporated herein by reference. Survival of bacteria may be determined by culturing the bacteria and counting colonies on appropriate selective or non-selective media.

In some embodiments, a composition is tested for sensitivity to bile acids. In some embodiments, testing for bile-acid resistance enhances the selection of bacterial compositions that will survive exposures to bile acid during transit through the GI tract. Bile acids are secreted into the small intestine and can, like pH, affect the survival of bacterial compositions. This may be tested by exposing the bacterial compositions to bile acids for the expected gut exposure time to bile acids. For example, bile acid solutions may be prepared at desired concentrations using 0.05 mM Tris at pH 9 as the solvent. After the bile acid is dissolved, the pH of the solution may be adjusted to 7.2 with 10% HCl. Bacterial compositions may be cultured in 2.2 ml of a bile acid composition mimicking the concentration and type of bile acids in the patient, 1.0 ml of 10% sterile-filtered feces media and 0.1 ml of an 18-hour culture of the given strain of bacteria. Incubations may be conducted for from 2.5 to 3 hours or longer. An alternative method for testing stability to bile acid is described in U.S. Pat. No. 4,839,281, incorporated herein by reference. Survival of bacteria may be determined by culturing the bacteria and counting colonies on appropriate selective or non-selective media.

In some embodiments, a composition is tested for sensitivity to antibiotics. In one embodiment, bacterial compositions may be chosen so that the bacterial constituents are sensitive to antibiotics such that if necessary they can be eliminated or substantially reduced from the patient's gastrointestinal tract by at least one antibiotic targeting the bacterial composition.

In some embodiments, compositions are tested for adherence to gastrointestinal cells. A method for testing adherence to gastrointestinal cells is described in U.S. Pat. No. 4,839,281, incorporated herein by reference.

### Subjects

Epidemiologic studies have shown that IBD patients, particularly UC, are at higher risk for CDI compared with non-IBD individuals (33, 48, 49, 50). Since CDI induces IBD flares and worsens disease outcome, CDI in IBD patients is recognized as a major clinical complication (33, 34, 35). During the development of embodiments of the technology provided herein, experiments were conducted that recapitulated an increased susceptibility to CDI seen in UC patients using the HMA mouse model described herein. Since it is known that the abundance of Phascolarctobacterium is decreased in IBD patients, both in UC and CD (51), the gut microbiotas derived from UC patients might have similar defects as those observed in IL-22-neutralized HMA mice (e.g., decreased abundance of Phascolarctobacterium and elevated levels of luminal succinate). Consistently, although the expression of IL-22 is up-regulated in the intestine of IBD patients (52, 53), there is evidence of possible defective IL-22 signaling in IBD, particularly UC, patients. For example, the development of IL-22-producing CD4+ T cells is impaired in UC patients (54, 55). Also, gut dysbiosis present in IBD impairs tryptophan metabolism, which is involved in the activation of the main IL-22 producer in the intestine (ILCs) through Ahr (56). In addition to impaired IL-22 production, IL-22BP, which blocks IL-22 bioactivity, is significantly up-regulated in UC, suggesting that IL-22 signaling is defective despite elevated IL-22 expression (57, 58). Moreover, SOCS3, which blocks the downstream targets of IL-22 signaling, is over-expressed in UC (59, 60), and polymorphisms in IL-22, IL22RA, and STAT3 are identified as risk genes for IBD (61, 62). Collectively, IL-22 signaling is likely compromised in UC patients. In this study, experiments were conducted and were collected indicating that the expression of IL22RA2 is significantly up-regulated in UC patients, and that IL22RA2 expression inversely correlates with the expression of host glycosyltransferases, MGAT4A and MGAT4B. Consistently, reduced host N-glycosylation has been reported in active UC patients (630. Based on these findings, it is possible that impaired IL-22 signaling in UC patients might cause abnormal host glycosylation, thus triggering gut dysbiosis (e.g., reduction in the abundance of Phascolarctobacterium spp).

The increased luminal succinate level is likely only one facet of the complex process that makes IBD patients more susceptible to CDI. While FMT is more effective than the administration of Phascolarctobacterium spp. to prevent CDI in UC-HMA mice, Phascolarctobacterium spp. treatment significantly reduced the availability of luminal succinate. Consistently, FMT reduced the levels of various metabolites that are known to foster the growth of C. difficile (e.g., amino acids) (47). Thus, Phascolarctobacterium spp. may be used with other bacterial strains that target distinct metabolic pathways related to C. difficile germination and growth (e.g., amino acids consumption, bile acid conversion). Although succinate might not be a single target to prevent CDI in IBD patients, it remains possible that these parameters (abundance of Phascolarctobacterium, the concentration of luminal succinate) can be utilized to screen and identify IBD patients at high risk for CDI. This finding also raises the question of whether modulation of IL-22 by current therapies for IBD could affect incidence of C. difficile colitis. It is possible that IL-23 inhibition by ustekinumab (Stelara), a medication currently approved for CD and in clinical trials for UC, could affect IL-22 expression and the risk of CDI. Current clinical data suggests no increased risk of infection with C. difficile in patients treated with ustekinumab, though the confidence intervals for this uncommon event remain wide at this time.

Restoration of the normal microbial community structure and function can prevent and/or cure diseases associated with gut dysbiosis. For example, FMT cures >95% of recurrent CDI (1). However, compared to CDI, clinical response to FMT in IBD is variable (64, 65, 66). This unresponsiveness of IBD patients to FMT is, at least in part, explained by impaired host glycosylation in IBD. In other words, IBD patients lack the nutritional niche required for a successful acquisition and retention of transplanted microbes. In this context, transplanted Phascolarctobacterium spp. may not efficiently colonize the gut of UC patients due to insufficiencies of the nutritional niche caused by impaired host glycosylation. In this case, the combination of Phascolarctobacterium and recombinant IL-22 (or co-inoculation with IL-22-inducing bacteria (67)) could maximize the colonization potential of transplanted Phascolarctobacterium in the gut.

Accordingly, in some embodiments, the subject has CDI. In some embodiments, the subject has a higher risk of having a CDI. In some embodiments, the subject has IBD. In some embodiments, the subject has UC. In some embodiments, the subject is in a hospital. In some embodiments, the subject has had, is having, and/or will have a surgery. In some embodiments, the subject has had, is having, and/or will have a surgery on a part, component, organ, etc. of the gastrointestinal tract. In some embodiments, the subject is colonized with C. difficile and/or has an increased risk of having a C. difficile infection. In some embodiments, the subject has an increased amount of C. difficile, e.g., increased relative to a normal subject, relative to a previous measurement in the same subject, relative to an amount agreed upon by those of ordinary skill in the art (e.g., medical practitioners), and/or relative to a normal amount reported in the medical literature. In some embodiments, the subject has a decreased amount of an organism from the genus Phascolarctobacterium such as P. faecium JCM30894 or P. succinatutens JCM16074, e.g., decreased relative to a normal subject, relative to a previous measurement in the same subject, relative to an amount agreed upon by those of ordinary skill in the art (e.g., medical practitioners), and/or relative to a normal amount reported in the medical literature. In some embodiments, the subject has a decreased amount of IL-22 production, e.g., decreased relative to a normal subject, relative to a previous measurement in the same subject, relative to an amount agreed upon by those of ordinary skill in the art (e.g., medical practitioners), and/or relative to a normal amount reported in the medical literature. In some embodiments, the subject has a decreased amount of gut glycans, e.g., decreased relative to a normal subject, relative to a previous measurement in the same subject, relative to an amount agreed upon by those of ordinary skill in the art (e.g., medical practitioners), and/or relative to a normal amount reported in the medical literature. In some embodiments, the subject has increased amount of gut succinate, e.g., increased relative to a normal subject, relative to a previous measurement in the same subject, relative to an amount agreed upon by those of ordinary skill in the art (e.g., medical practitioners), and/or relative to a normal amount reported in the medical literature.

In some embodiments, subjects for treatment are selected based on a symptom of CDI and/or a molecular genetic indicator that treatment is needed. In some embodiments, particular bacterial compositions are selected for individual patients or for patients with particular profiles (e.g., patient classes). For example, in some embodiments, a subject has decreased IL-22 in the gut. As another example, 16S sequencing may be performed for a given patient to identify the bacteria present in his or her microbiota. The sequencing may either profile the patient's entire microbiome using 16S sequencing (e.g., to the family, genus, or species level), a portion of the patient's microbiome using 16S sequencing, or it may be used to detect the presence or absence of specific candidate bacteria that are biomarkers for health or a particular disease state, such as markers of multi-drug resistant organisms or specific genera of concern. In some embodiments, sequencing is used to detect the presence or an increase (relative to normal or relative to a previously measured amount) of C. difficile. In some embodiments, sequencing is used to detect the absence or an decrease (relative to normal or relative to a previously measured amount) of an organism from the genus Phascolarctobacterium, such as P. faecium JCM30894 or P. succinatutens JCM16074. Based on the biomarker data, a particular composition may be selected for administration to a patient to supplement or complement a patient's microbiota in order to restore health or treat or prevent disease. In another embodiment, patients may be screened to determine the composition of their microbiota to determine the likelihood of successful treatment.

Methods of Diagnosis and Treatment

In some embodiments, the technology provides methods of diagnosing a subject. In some embodiments, the technology provides methods of treating a subject. In some embodiments, the technology provides a method of identifying a subject in need of a treatment for a Clostrodioides difficile infection (CDI). In some embodiments, method comprise administering a composition as described herein to a subject.

In some embodiments, methods for identifying a subject in need of treatment for CDI comprises measuring an increase or decrease in one or more biomarkers. In some embodiments, methods for identifying a subject in need of treatment for CDI comprises detecting the presence or absence of a biomarker. As discussed herein, the presence, absence, increase, and/or decrease in several biomarkers indicates the presence of dysbiosis in the gut, a CDI, and/or conditions that promote a CDI in a subject. In some embodiments, methods comprise detecting a decrease and/or absence of an organism from the genus Phascolarctobacterium, such as P. faecium or P. succinatutens, in the gut. In some embodiments, methods comprise detecting a decrease and/or absence of glycan in the gut. In some embodiments, methods comprise detecting a decrease and/or absence of IL-22 in the gut. In some embodiments, methods comprise detecting a decrease and/or absence of MGAT4A expression or MGAT4A protein in the gut. In some embodiments, methods comprise detecting a decrease and/or absence of MGAT4B expression or MGAT4B protein in the gut. In some embodiments, methods comprise detecting an increase and/or presence of succinate in the gut. In some embodiments, methods comprise detecting an increase and/or presence of C. difficile in the gut.

In some embodiments, methods comprise detecting one or more (e.g., 1, 2, 3, 4, 5, 6, or 7) of the following in combination: decrease and/or absence of an organism from the genus Phascolarctobacterium, such as P. faecium or P. succinatutens, in the gut; decrease and/or absence of glycan in the gut; decrease and/or absence of IL-22 in the gut; decrease and/or absence of MGAT4A expression or MGAT4A protein in the gut; decrease and/or absence of MGAT4B expression or MGAT4B protein in the gut; increase and/or presence of succinate in the gut; and/or detecting an increase and/or presence of C. difficile in the gut.

In some embodiments, methods comprise detecting a microorganism (e.g., an organism from the genus Phascolarctobacterium (e.g., P. faecium and/or P. succinatutens) and/or C. difficile) using solid or liquid culture. In some embodiments, a solid or liquid culture is inoculated by a sample from a subject (e.g., a sample from the subject gut and/or feces). In some embodiments, methods comprise detecting a microorganism by detecting a nucleic acid from the microorganism. For example, methods comprise using probe hybridization, nucleic acid amplification (e.g., polymerase chain reaction), or other nucleic acid detection techniques. In some embodiments, methods comprise detecting and/or quantifying an amount of a particular nucleic acid, e.g., a ribosomal RNA (e.g., a 16S ribosomal RNA) indicative of the presence of the microorganism. In some embodiments, a quantitative method is used to quantify the amount of one or more organisms in a sample from a subject. In some embodiments, methods comprise use of quantitative PCR (qPCR). In some embodiments, methods comprise obtaining (e.g., extracting) genomic DNA from a sample from a subject, e.g., using commercially-available kit (e.g., such as the Mo Bio POWERSOIL-htp 96 Well Soil DNA Isolation Kit (Mo Bio Laboratories, Carlsbad, Calif.), Mo Bio POWERSOIL DNA Isolation Kit (Mo Bio Laboratories, Carlsbad, Calif.), or the QIAamp DNA Stool Mini Kit (QIAGEN, Valencia, Calif.) according to the manufacturer instructions).

In some embodiments, methods comprise sequencing a gene encoding a ribosomal RNA (e.g., a 16S ribosomal RNA) obtained from a sample from a subject to identify the organisms present in the subject's gut (e.g., in the subject microbiota). The sequencing can either profile the entire microbiome of a subject using 16S sequencing (to the family, genera, or species level), a portion of the subject microbiome using 16S sequencing, or it can be used to detect the presence or absence of specific candidate bacteria that are biomarkers for health or a particular disease state, such as an organism from the genus Phascolarctobacterium (e.g., P. faecium and/or P. succinatutens) and/or C. difficile). Based on the biomarker data, in some embodiments, methods comprise selecting a particular composition for administration to a subject to supplement or complement the subject's microbiota to restore health or treat or prevent disease. In some embodiments, methods comprise measuring the relative abundance of microorganisms in a sample, for example, using Roche/454 pyrosequencing or Illumina sequencing for 16S rRNA gene sequencing. This approach, combined with multiplexing, produces thousands of 16S rRNA sequences per sample. Microbiome sequencing techniques are further reviewed in, e.g., Grice, E A, and Segre J A. Annu Rev Genomics Hum Genet. 2012; 13: 151-170, incorporated herein by reference. Relative abundance can additionally be measured using, e.g., using amplicons for microbiologic or microbial products and/or other gene-level targets.

Methods of detecting proteins (e.g., IL-22 and products of the MGAT4A and/or MGAT4B genes) include, e.g., are known to one of ordinary skill in the art. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody. For example, the antibody can be labeled with a detectable marker whose presence and location in the subject is detected by standard imaging techniques.

In some embodiments, methods comprise screening subjects to determine the composition of the microbiota to determine the likelihood of successful treatment.

In some embodiments, the technology relates to predicting or determining the likelihood of CDI or recurrence of CDI in a subject. In some embodiments, such a prediction can guide prophylactic and subsequent therapeutic treatment with a composition as described herein. In some embodiments, detecting the presence of C. difficile comprises use of microbiologic, toxin ELISA, or molecular methods to predict likelihood of infection or recurrence. The presence and/or levels of various markers can be compared, for example, to a reference to determine likelihood of infection or recurrence. The reference can be, for example, a sample from a healthy individual, or as the case may be a sample from an individual with active C. difficile infection.

In some embodiments, methods comprise obtaining a sample from a subject who is at risk of having, has, or has previously had at least one C. difficile infection. A sample can be taken from a subject who has never had a C. difficile infection, but who is at risk of having a C. difficile infection. A stool sample can be collected using standard techniques, e.g., passing stool directly into a clean, dry container. In some embodiments, diagnosing a subject comprises obtaining at least one sample from the subject. However, repeated sampling can also be performed. For example, a sample can be taken from a subject once a day, once a week, twice a month, once a month, or every 3 months following a C. difficile infection to assess the risk of a recurrent C. difficile infection, or a sample can be taken from a subject once a year following a C. difficile infection to assess the risk of a recurrent C. difficile infection.

In some embodiments, a sample is taken from a subject who has not previously been treated with antibiotics to treat a C. difficile infection. Alternatively, in some embodiments, a sample is taken from a subject who has been treated with antibiotics to treat a C. difficile infection. The sample can be taken from the subject before, during, or after administration of antibiotics to treat a C. difficile infection. A sample can be taken from a subject before, during, and after administration of a composition as described herein (e.g., a sample is taken from a subject during and after administration of a composition as described herein).

In some embodiments, the technology provides a method of treating and/or preventing a CDI comprising testing a subject, wherein said testing comprises determining that a subject has a decrease and/or absence of an organism from the genus Phascolarctobacterium, such as P. faecium or P. succinatutens, in the gut; decrease and/or absence of glycan in the gut; decrease and/or absence of IL-22 in the gut; decrease and/or absence of MGAT4A expression or MGAT4A protein in the gut; decrease and/or absence of MGAT4B expression or MGAT4B protein in the gut; increase and/or presence of succinate in the gut; and/or detecting an increase and/or presence of C. difficile in the gut; and administering a therapeutic composition as described herein to the subject. In some embodiments, the step of testing the subject comprises obtaining or having obtained a biological sample from the subject and performing or having performed an assay on the sample obtained from the subject to determine/measure a decrease and/or absence of an organism from the genus Phascolarctobacterium, such as P. faecium or P. succinatutens; decrease and/or absence of glycan; decrease and/or absence of IL-22; decrease and/or absence of MGAT4A expression or MGAT4A protein; decrease and/or absence of MGAT4B expression or MGAT4B protein; increase and/or presence of succinate; and/or detecting an increase and/or presence of C. difficile.

In some embodiments, a subject is tested to assess the presence, the absence, the level of a disease, and/or the likelihood of having a disease (e.g., CDI or conditions in the gut that promote a CDI (e.g., dysbiosis)), e.g., by assaying or measuring a biomarker, a metabolite, a physical symptom, an indication, etc. (e.g., one or more of decrease and/or absence of an organism from the genus Phascolarctobacterium, such as P. faecium or P. succinatutens; decrease and/or absence of glycan; decrease and/or absence of IL-22; decrease and/or absence of MGAT4A expression or MGAT4A protein; decrease and/or absence of MGAT4B expression or MGAT4B protein; increase and/or presence of succinate; and/or detecting an increase and/or presence of C. difficile), to determine the risk of or the presence of disease and thereafter the subject is treated with a composition as described herein (e.g., a composition comprising an organism from the genus Phascolarctobacterium, such as P. faecium or P. succinatutens, and/or IL-22 or a composition that promotes IL-22 production in the gut) based on the outcome of the test.

In some embodiments, a patient is tested, treated, and then tested again to monitor the response to therapy. In some embodiments, cycles of testing and treatment may occur without limitation to the pattern of testing and treating (e.g., test/treat, test/treat/test, test/treat/test/treat, test/treat/test/treat/test, test/treat/treat/test/treat/treat, etc.), the periodicity, or the duration of the interval between each testing and treatment phase.

In some embodiments, methods of treatment comprise administering a composition that reduces succinate in the gut, e.g., a small molecule, an enzyme that degrades succinate, an antibody that binds succinate, a molecule that sequesters succinate, etc. In some embodiments, an antibiotic is administered that decreases and/or eliminates a succinate producer in the gut, e.g., Bacteroides spp. In some embodiments, a subject is treated with IL-22. In some embodiments, a subject is treated with N-acetylglucosamine.

### Kits

In some embodiments, the technology relates to kits, e.g., kits comprising a composition or a plurality of compositions as described herein (e.g., a composition comprising an organism from the genus Phascolarctobacterium, such as P. faecium or P. succinatutens, and/or IL-22 or a composition that promotes IL-22 production in the gut). In some embodiments, kits comprise one or more discrete compositions comprising a probiotic as described herein, e.g., comprising an organism from the genus Phascolarctobacterium, such as P. faecium or P. succinatutens, and/or IL-22 or a composition that promotes IL-22 production in the gut. In some embodiments, kits comprise a plurality of doses of a composition. For example, the kit may comprise a daily, weekly, monthly, or other periodic dose of a composition as described herein. As an illustrative example, a kit may comprise 7 discrete compositions comprising a probiotic as described herein (7 daily doses) or 30 discrete compositions comprising the probiotic (30 daily doses). In certain embodiments, the kits are configured to facilitate dosing compliance.

In some embodiments, the technology provides kits for use by a medical professional for administration of a composition described herein to a subject. For example, in some embodiments, kits comprise one or more doses of a composition described herein, a composition for diluting one or more doses of the composition, and/or a component for administration of the composition to a subject (e.g., a component for oral and/or rectal administration). In some embodiments, kits comprise a flavoring and/or thickener to provide a composition for oral ingestion.

In some embodiments, the technology provides kits suitable for administering a composition described herein to an animal. In some embodiments, kits comprise in separate containers in a single package at least one composition as described herein. In some embodiments, a kit contains a composition as described herein and other components in amounts sufficient to provided beneficial amounts of the composition to the animal (e.g., subject). In some embodiments, a composition according to the technology is mixed with other components (e.g., food compositions) just prior to consumption by a subject. The kits may contain the kit components in any of various combinations and/or mixtures.

### Systems

In some embodiments, the technology relates to systems for treating a CDI. In some embodiments, a system comprises a composition or a plurality of compositions as described herein (e.g., a composition comprising an organism from the genus Phascolarctobacterium, such as P. faecium or P. succinatutens, and/or IL-22 or a composition that promotes IL-22 production in the gut). In some embodiments, systems comprise one or more discrete compositions comprising a probiotic as described herein, e.g., comprising an organism from the genus Phascolarctobacterium, such as P. faeciumor P. succinatutens, and/or IL-22 or a composition that promotes IL-22 production in the gut.

In some embodiments, the technology provides systems for use by a medical professional for administration of a composition described herein to a subject. For example, in some embodiments, kits comprise one or more doses of a composition described herein, a composition for diluting one or more doses of the composition, and/or a component for administration of the composition to a subject (e.g., a component for oral and/or rectal administration), e.g., a pill, tablet, capsule, osmotic delivery system, suppository, enema, osmotic delivery system, suppository, enema, tube, or other component known in the art for administering a composition to the gut of a subject. In some embodiments, systems comprise an enteric coated drug delivery device or enteric coated composition, which refer to any drug delivery method that can be administered orally but is not degraded or activated until the device enters the intestines (e.g., a form for delivery comprising a coating or encapsulation that is degraded using, e.g., pH-dependent means, permitting protection of the delivery device and the composition to be administered to the gut until the device reaches the gut).

In some embodiments, systems comprise a component for obtaining a sample from a subject, e.g., from the gut. In some embodiments, the component for obtaining a sample is configured to obtain a fecal sample, e.g., a bucket, tube, net, or card for obtaining a fecal sample from a subject. In some embodiments, systems comprise components for isolating microorganisms from a sample, e.g., for culture and identification and/or quantification of one or more microorganisms from the sample. In some embodiments, systems comprise components for isolating nucleic acids from a sample for molecular characterization of nucleic acids (e.g., amplification of ribosomal RNA genes, sequencing (e.g., sequencing ribosomal RNA genes or other phylogenetically useful nucleic acids)). In some embodiments, systems comprise components for amplification of nucleic acid, e.g., a polymerase and oligonucleotide primers targeting a phylogenetically useful nucleic acid (e.g., a ribosomal RNA gene (e.g., a 16S rRNA gene)).

In some embodiments, systems comprise an oligonucleotide primer, an antibody, and/or a diagnostic apparatus for performing one or more diagnostic methods as described herein. Some embodiments of the technology provided herein further comprise functionalities for collecting, storing, and/or analyzing data. For example, in some embodiments systems comprise a processor, a memory, and/or a database for, e.g., storing and executing instructions, analyzing data, performing calculations using the data, transforming the data, and/or storing the data. Moreover, in some embodiments a processor is configured to receive diagnostic data (e.g., test results) and suggest a course of treatment (e.g., an amount, dose, and/or dosing schedule of one or more compositions as described herein).

### Uses

In some embodiments, the present technology finds use in manufacturing a food composition comprising a composition described herein (e.g., a composition comprising an organism from the genus Phascolarctobacterium, such as P. faecium or P. succinatutens, and/or IL-22 or a composition that promotes IL-22 production in the gut). In some embodiments, the technology finds use in promoting the health or wellness of a subject comprising administering to a subject a health or wellness promoting amount of a composition described herein. In some embodiments, the technology finds use in methods of treating a subject in need of a treatment for CDI wherein a composition as described herein is administered to the subject in amounts of from approximately one to about twenty billion colony forming units (CFUs) per day, preferably from about 10 million to about 10 billion live bacteria per day.

Although the disclosure herein refers to certain illustrated embodiments, it is to be understood that these embodiments are presented by way of example and not by way of limitation.

### EXAMPLES

The resident gut microbiota plays a role in protecting a host from infectious organisms. Colonization of the gut by microbiota is known to promote various host immune responses that combat pathogens and attenuate pathogen-induced inflammation. In turn, immune responses shape the gut microbiota. The bi-directional tuning that occurs between the host and the microbiota in the context of host defense remain largely unknown. Accordingly, experiments were conducted during the development of the technology provided herein to evaluate protection against CDI by host microbiota and microbiota-induced immune responses in the host. In particular, data collected during the experiments described herein indicate that IL-22 participates in feedback regulation of the gut microbiota. IL-22-induced host glycosylation governs the growth of certain protective members of the gut microbiota, such as Phascolarctobacterium. IL-22 normalizes the composition of the microbiota and influences its metabolic activities to prevent opportunistic colonization and/or expansion of C. difficile in the gut.

### Materials and Methods

Mice. Specific pathogen-free (SPF) mice were housed in the Animal Facility at the University of Michigan. GF C57BL/6 and GF Rag1^{-/-} mice were housed in the Germ-Free Animal Facility at the University of Michigan. GF mice were maintained in flexible film isolators and their GF status was checked weekly by aerobic and anaerobic culture. The absence of the microbiota was verified by microscopic analysis of stained cecal contents to detect unculturable contamination. Stool samples obtained from patients with UC and healthy control subjects (University of Michigan Institutional Review Board approved protocol-IBD Databank, HUM00041845) were used to generate HMA mice. Written informed consent forms were obtained from all subjects before sample collection. UC patients and control subjects were not treated with any antibiotics for at least 3 months before sample collection and had no history of intestinal bacterial infections with Clostridium difficileor other infections such as hepatitis B virus, hepatitis C virus, or human immunodeficiency virus. Patients were histologically and endoscopically diagnosed with UC prior to enrollment. Patient information is provided in Table 3. Collected stool samples were stored at −80° C. until use. Before inoculation, stool samples were diluted 1:10 with pre-reduced phosphate-buffered saline under anaerobic conditions. Diluted stool samples then were passed through a 100-μm cell strainer and used to inoculate GF C57BL/6 mice or GF Rag1^{-/-} mice orally (100 μL per mouse).

TABLE 3
Patient information

| Patient | | | | Disease | |
|---|---|---|---|---|---|
| ID | Disease | Sex | Age | activity | Special note |
| UC#01 | UC | F | 46 | Inactive | |
| UC#02 | UC | F | 30 | Inactive | Colectomy with |
| | | | | (high ESR, | J pouch, PSC, on |
| | | | | normal CRP) | Chemo at time of |
| | | | | | sample collection |
| UC#03 | UC | M | 18 | Active | |
| | | | | (elevated CRP) | |
| UC#04 | UC | M | 25 | Active | |
| | | | | (elevated CRP) | |
| UC#05 | UC | F | 47 | Active | |
| | | | | (elevated CRP) | |

Human microbiota-associated mice. HMA mice were housed in positive-pressure individually ventilated cages (IVCs) (ISOcage P; Techniplast, West Chester, PA) to prevent cross-contamination and maintain gnotobiotic status (68, 69). All mice were fed a sterilized rodent breeder diet 5013 (LabDiet, St. Louis, MO). 8 to 16-week-old female and male mice were used in all experiments. All animals were handled in accordance with the protocols approved by the Institutional Animal Care and Use Committee (IACUC) at the University of Michigan. To attain robust C. difficile infection in HC-HMA mice, animals were treated with a five-antibiotic cocktail. Kanamycin (0.4 mg/mL), gentamicin (0.035 mg/mL), colistin (850 U/mL), metronidazole, (0.215 mg/mL), and vancomycin (0.045 mg/mL) were administered to HC-HMA mice in drinking water for 5 days, followed by an intraperitoneal injection of clindamycin (10 mg/kg) 24 hours later. The mice were infected with C. difficile 24 hours after intraperitoneal injection (28).

Bacterial strain and C. difficile infection of mice. C. difficile VPI 10463 (ATCC 43255) spores were heat treated for 20 minutes at 65° C. to kill vegetative bacilli. Wild-type JIR8094 and Cd-CD2344—(32) were grown overnight in brain heart infusion medium supplemented with 100 mg/L L-cysteine and 5 mg/mL yeast extract (BHIS). Mice were infected with C. difficile VPI10463 spores (103 spores per mouse) or an overnight culture of JIR8094/Cd-CD2344—(OD₆₀₀=0.5-1.0, 200 μL per mouse) by oral gavage. Stool samples were collected from C. difficile-infected mice on days 1, 3, 7, 10, and 14 post challenge. The samples were then serially diluted and plated on TCCFA selective plates to quantify the number of C. difficile spores under anaerobic conditions. For the αIL-22 antibody treatment, HC-HMA Rag1^{-/-} mice were injected intraperitoneally twice before C. difficile inoculation (3 and 5 days prior to infection) and 3 times per week post infection with the αIL-22 antibody (150 μg/mouse per dose) (Genentech, South San Francisco, CA, USA) or an equivalent amount of the isotype-matched control antibody (Genentech). For the IL-22-Fc fusion protein treatment, GF mice were injected intraperitoneally once before Phascolaectobacterium spp. inoculation (1 day prior to inoculation) and twice post inoculation (2 and 5 days) with IL-22-Fc (100 μg/mouse per dose) (Genentech, South San Francisco, CA, USA) or an equivalent amount of the isotype-matched control protein (Genentech). C. difficile-infected UC-HMA mice were transplanted with a healthy human-derived microbiota (fecal microbiota transplantation; FMT) or Phascolarctobacterium (10⁶ CFU/mouse) once before C. difficile inoculation (3 days prior to infection) and 4 times post inoculation (1, 3, 7, and days post infection). Seven days or 2 weeks post C. difficile inoculation, HMA mice were sacrificed. Cecum and colon tissues were collected and fixed with 4% paraformaldehyde. Fixed tissues were then processed, embedded, sectioned, and stained with hematoxylin and eosin (H&E). Histological assessment was performed by a pathologist in a blinded fashion at the Unit for Laboratory Animal Medicine in vivo Animal Core. A 4-point scale was used to denote the severity of inflammation (0, none; 1, minimal multifocal inflammation [few foci]; 2, moderate multifocal inflammation [numerous foci]; 3, severe multifocal coalescing inflammation; and 4, same as a score of 3 with abscesses or extensive mural involvement), the edema scores (0, none; 1, mild focal or multifocal edema, minimal submucosal expansion (<2); 2, moderate focal or multifocal edema, moderate submucosal expansion (2-3); 3, severe multifocal to coalescing inflammation; and 4, same as a score of 3 with diffuse submucosal expansion), and the epithelial score (0, none; 1, mild, multifocal, superficial damage; 2, moderate, multifocal, superficial damage; 3, severe, multifocal to coalescing mucosal damage±pseudomembrane±ulcer; 4, same as a score of 3 with significant pseudomembrane or ulcer formation). Each variable was then summed to obtain the overall score.

Quantitative Real-Time PCR. RNA was extracted with E.Z.N.A. Total RNA Kit (Omega Bio-tek, Norcross, GA) according to the manufacturer instructions. RNA was reverse transcribed using a High Capacity RNA-to-cDNA Kit (Applied Biosystems, Foster, CA) and cDNA was then used for quantitative PCR analysis using a SYBR Green Gene-Expression Assay on an ABI 7900HT analyzer (ABI 7900HT analyzer). The following primer sets were used for amplification. Sequences are provided 5′ to 3′.

| | | SEQ ID |
|---|---|---|
| Primer name | Sequence (5′ to 3′) | NO: |
| Actb-F | AAGTGTGACGTTGACATCCG | 1 |
| Actb-R | GATCCACATCTGCTGGAAGG | 2 |
| Il-22-F | TTTCCTGACCAAACTCAGCA | 3 |
| Il-22-R | TCTGGATGTTCTGGTCGTCA | 4 |
| Reg36-F | CTCTCCTGCCTGATGCTCTT | 5 |
| Reg36-R | GTAGGAGCCATAAGCCTGGG | 6 |
| Reg3γ-F | TCAGGTGCAAGGTGAAGTTG | 7 |
| Reg3γ-R | GGCCACTGTTACCACTGCTT | 8 |
| Mgat4a-F | GCGACAGACAGAAGGCAAACC | 9 |
| Mgat4a-R | CCGACAGAGACGAGTGTAGGC | 10 |
| Mgat4b-F | AGGTGACGTGGTGGACATTT | 11 |
| Mgat4b-R | GCTTCAGGCTCTCTTGCTCA | 12 |
| Mgat5-F | GGAAATGGCCTTGAAAACACA | 13 |
| Mgat5-R | CAAGCACACCTGGGATCCA | 14 |
| St6gal1-F | TGCGTGTGGAAGAAAGGGAGC | 15 |
| St6gal1-R | CTCCTGGCTCTTCGGCATCTG | 16 |
| Eubacteria-F | ACTCCTACGGGAGGCAGCAGT | 17 |
| Eubacteria-R | ATTACCGCGGCTGCTGGC | 18 |
| P. faecium-F | CCTTTAGACGGGGACAACATTC | 19 |
| P. faecium-R | ATCGCCTTGGTAGTCCGTTACA | 20 |
| P. succinatutens-F | AGCAATCTCGCATGAGGATGCTGT | 21 |
| P. succinatutens-R | GCCGTGGCTTATTCGTTTACTACCG | 22 |
| Tnf (genomic DNA)-F | CAACCCTTATTCTCGCTCACA | 23 |
| Tnf (genomic DNA)-R | CTCCACACTCTCCTCCACCT | 24 |

Microbiome analysis. Genomic DNA was extracted using a modified Qiagen DNeasy Blood and Tissue kit protocol (Qiagen, Valencia, CA). Modifications included the following steps: (1) UltraClean fecal DNA bead tubes (Mo Bio Laboratories, Inc, West Carlsbad, CA) and a Mini-Beadbeater-16 (BioSpec Products, Inc, Bartlesville, OK) were used to homogenize samples (1.5 minutes); (2) the amount of buffer ATL used in the initial steps of the protocol was increased (from 180 to 400 μL); (3) the volume of proteinase K was increased (from 20 to 40 μL); and (4) the amount of buffer AE used to elute DNA at the end of the protocol was decreased (from 200 to 75 μL). 16S rRNA sequencing was done by the University of Michigan Medical School Host Microbiome Initiative core facility and processed using the MiSeq Illumina sequencing platform. 16S ribosomal RNA (rRNA) gene libraries were constructed using primers specific for the V4 region. Sequences were curated using the community-supported software program mothur (v.1.33) (70) and by following the steps outlined in the MiSeq SOP (www.mothur.org/wiki/MiSeq_SOP) (71). Sequences were assigned to operational taxonomic units (OTUs) using a cutoff of 0.03 and classified against the Ribosomal Database Project (RDP) 16S rRNA gene training set (version 9) using a naïve Bayesian approach with an 80% confidence threshold. Curated OTU sequence data was converted to relative abundance±standard error of the mean. Within-community diversity (α-diversity) was calculated using Shannon diversity index (H′) and OTU Richness. Between-community diversity (β-diversity) was determined using the Yue and Clayton (θYC) dissimilarity distance metric. Non-metric multidimensional scaling (NMDS) was used to ordinate the β-diversity data. An analysis of molecular variation (AMOVA) was used to test for significant differences in the community structure using 10,000 permutations. Linear discriminant analysis effect size (LEfSe) (72) was used to test which functional pathways were differentially abundant, biologically consistent, and had the greatest effect size.

Metabolome analysis. Capillary electrophoresis time-of-flight mass spectrometry (CE-TOFMS)-based metabolome analysis was conducted as described previously with some modifications (73). In brief, fecal samples were lyophilized using a VD-800R lyophilizer (TAITEC, Saitama, Japan) for 24 hours. Freeze-dried feces were disrupted with 3.0-mm Zirconia Beads (Biomedical Science, Tokyo, Japan) by vigorous shaking (1,500 rpm for 10 minutes) using a Shake Master (Biomedical Science, Tokyo, Japan). Fecal metabolites were extracted using a methanol:chloroform:water extraction protocol. CE-TOFMS experiments were performed using an Agilent CE System, Agilent G3250AA LC/MSD TOF System, Agilent 1100 Series Binary HPLC Pump, Agilent G1603A CE-MS adapter, and Agilent G1607A CE-ESI-MS Sprayer Kit (Agilent Technologies, Santa Clara, CA). In-house software (MasterHands) was used for data processing, peak annotation, and quantification. For luminal succinate analysis, frozen stool samples were sent to the Michigan Regional Comprehensive Metabolomics Resource Core. Liquid chromatography-mass spectrometry (LC-MS)-based metabolome analysis was conducted as described previously with some modifications (74). In brief, fecal samples were transferred to pre-weighed microtubes and the weight recorded for future normalization. A 0.5-mL mixture of methanol, chloroform, and water (8:1:1) containing 13C4-labeled succinate was added to the tube. Next, the mixture was subjected to probe sonication until completely homogenized, incubated at 4° C. for 10 minutes, and then centrifuged at 14,000 RMP for 10 minutes and at 4° C. The extracts were removed and placed into autosampler vials for mass spectrometric analysis. 10 μl of each sample was removed and pooled in a separate autosampler vial for quality control purposes. A series of calibration standards were prepared in parallel with the samples to quantify metabolites. LC-MS analysis was performed on an Agilent system comprising a 1260 UPLC module coupled with a 6520 Quadrupole-Time-of-flight (QTOF) mass spectrometer (Agilent Technologies, Santa Clara, CA). Metabolites were separated on a 150×1 mm Luna NH₂ hydrophilic interaction chromatography column (Phenomenex, Torrance, CA) using 10 mM ammonium acetate in water, adjusted to pH 9.9 with ammonium hydroxide, as mobile phase A, and acetonitrile as mobile phase B. The flow rate was 0.075 mL/min and the gradient was linear 20% to 100% mobile phase A over 15 minutes, followed by isocratic elution at 100% mobile phase A for 5 minutes. The system was returned to starting conditions (20% mobile phase A) in 0.1 minute and held there for 10 minutes to allow for column re-equilibration before injecting another sample. The mass spectrometer was operated in ESI mode according to previously published conditions. Data were processed using MassHunter Quantitative analysis version B.07.00. Succinate was normalized to its isotopically labeled internal standard and quantified using 2 replicated injections of 5 standards to create a linear calibration curve with accuracy better than 80% for each standard. Other compounds in the analysis were normalized to the nearest internal standard and the peak areas were used for differential analysis between groups.

Ex vivo growth of C. difficile (JIR8094 and Cd-CD2344⁻). Overnight cultures of JIR8094 and Cd-CD2344⁻-were diluted 5000-fold with a minimal medium (10 g/L peptone, 10 g/L beef extract, 3 g/L yeast extract, 1.55 g/L NaH₂PO₄·H₂O, 5.95 g/L Na₂HPO₄) supplemented with 1% glucose, 0.1% succinate, or 100 μg/mL UC luminal content. A handheld spectrophotometer (Biochrom US, Holliston, MA) was used for optical density (OD₆₀₀) measurements.

Ex vivo germination and growth of C. Difficile (VPI10463). Mouse cecal contents from HC-HMA and UC-HMA mice were quantified inside an anaerobic chamber and diluted 10-fold with PBS. Heat-treated (65° C., 20 minunes) C. difficile spores (10³ spores) were added to the cecal contents, which were then incubated anaerobically at 37° C. for 6 hours. After incubation, bacterial samples were serially diluted and plated on TCCFA plates to quantify germination and growth of C. difficile.

In vitro growth of Phascolarctobacterium. P. faecium JCM30894 and P. succinatutens JCM16074 (JCM, Ibaragi, Japan) were grown overnight in a peptone-yeast extract medium supplemented with 80 mM sodium succinate (PYS) under anaerobic conditions (30). The overnight culture of Phascolarctobacterium spp. was diluted 100-fold with PYS medium supplemented with 300 μg of colonic mucus from SPF-WT mice or Il22^{-/-} mice. Briefly, colonic mucus was scraped from colonic walls into HEPES-Hanks' buffer (8.0 g/L NaCl, 0.4 g/L KCl, 0.05 g/L CaCl₂·H₂O, 0.35 g/L KH₂PO₄, 0.2 g/L MgSO₄·7H₂O and 2.6 g/L HEPES, pH 7.4). Contaminating epithelial cells and membranes were removed by centrifugation, once at 12,000×g for 10 minutes at 4° C. and once at 27,000×g for 15 minutes at 4° C. Colonic mucus in HEPES-Hanks' buffer was stored at −80° C. until use (75). Bacterial DNA was collected and bacterial copy number was quantified by qPCR 24 or 48 hours after incubation.

Gene Expression Omnibus (GEO) accession number. Publically available gene expression data used in this study are available from the NCBI Gene Expression Omnibus under the following accession codes: GSE75214 (37), GSE16879 (38), and GSE73661)39).

Statistical Analyses. Statistical analyses were performed using GraphPad Prism software version 5.0 (GraphPad Software Inc.). Statistical tests used for the analysis of data are identified in the legend of each figure. Differences of P<0.05 were considered significant.

### Example 1—Human microbiotas protect germ-free mice from CDI

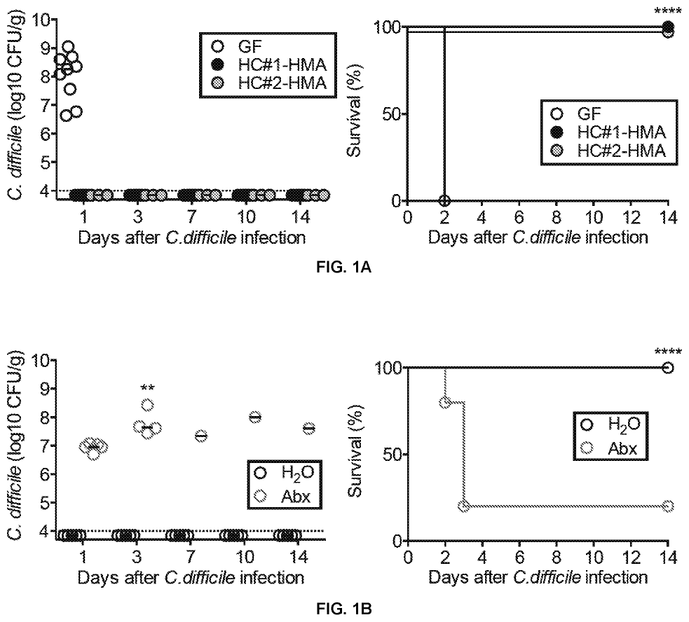

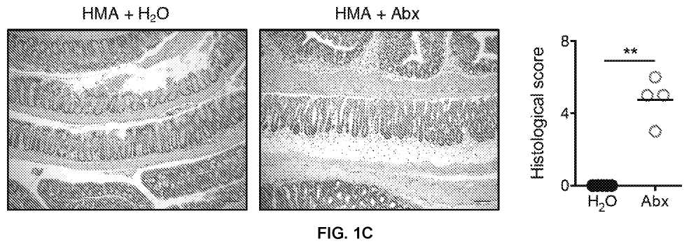

During the development of embodiments of the technology provided herein, experiments were conducted to test the protective role of human-derived microbiotas against CDI. GF mice were colonized with gut microbiotas isolated from two healthy controls (HC). HC human microbiota-associated (HC-HMA) mice were rested for two weeks to allow for full reconstitution, as previously reported (26, 27). HC-HMA mice were then infected with C. difficile strain VPI 10463. As a control, GF mice without microbiota reconstitution were also infected with C. difficile. C. difficile colonized control GF mice 1 day after infection and the following day all mice succumbed to CDI (FIG. 1A). Consistent with a previous report (28), HC-HMA mice were completely protected against CDI and C. difficile was unable to colonize these mice (FIG. 1A). To confirm the importance of the gut microbiota, HC-HMA mice were treated with a broad-spectrum antibiotic cocktail (e.g., comprising kanamycin, gentamicin, colistin, metronidazole, vancomycin, and clindamycin) (28). As expected, marked C. difficile colonization (>10⁷ to 10⁸ CFU/g feces) was observed in antibiotic-treated HC-HMA mice starting on day 1 post-CDI and the majority of mice succumbed to CDI (FIG. 1B). Consistent with fecal pathogen burden data, antibiotic-treated HC-HMA mice developed inflammatory pathology in the colon, while no overt inflammation was observed in control HC-HMA mice (FIG. 1C). These data indicate that human-derived microbiotas confer colonization resistance against C. difficile in mice.

Example 2—Microbiota-induced IL-22 prevents CDI

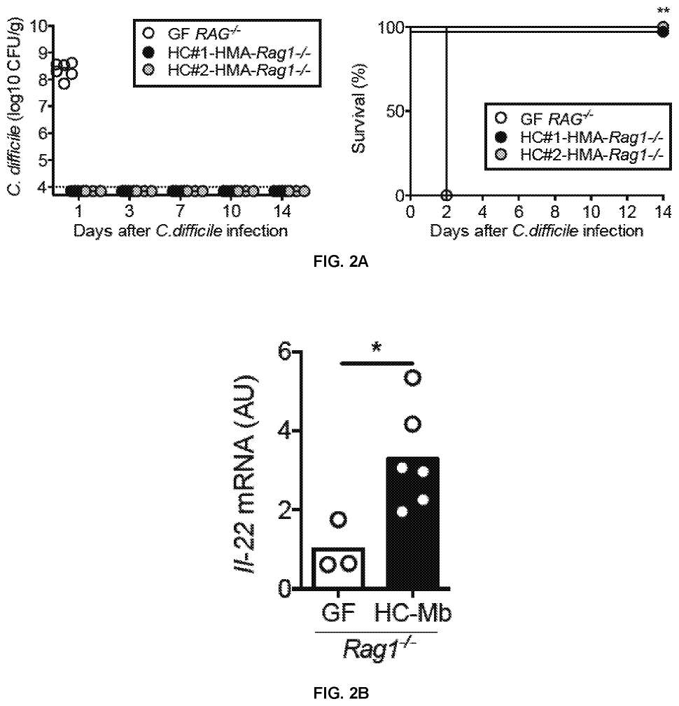

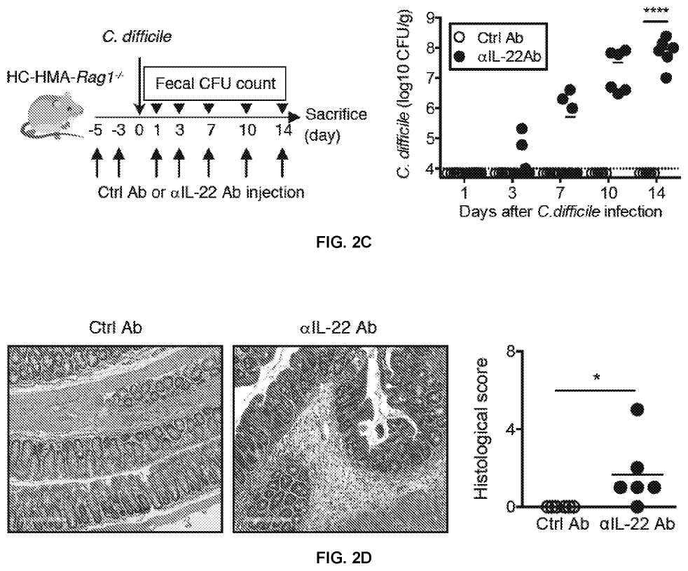

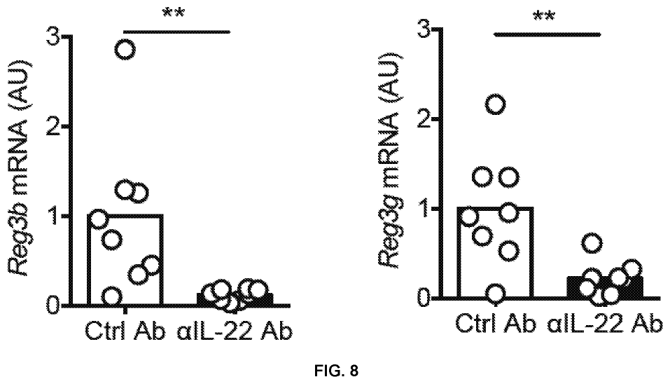

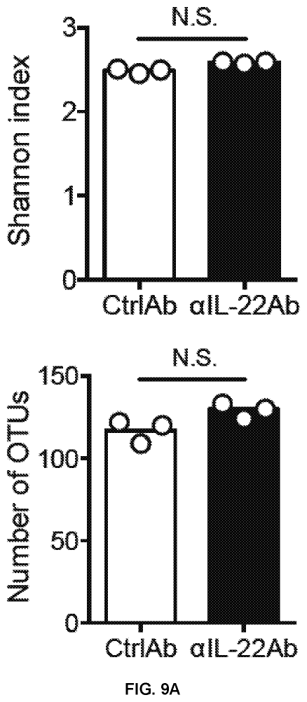

During the development of embodiments of the technology provided herein, experiments were conducted in which Rag1^{-/-} mice, which lack both T and B cells, were used to address the role of host immunity in human microbiota-conferred colonization resistance against C. difficile. GF Rag1^{-/-} mice were colonized with human microbiotas from two HCs and allowed to reconstitute for 2 weeks (HC-HMA-Rag1^{-/-} mice). GF Rag1^{-/-} and HC-HMA-Rag1^{-/-} mice were then infected with C. difficile. As shown in FIG. 2A, C. difficile colonized control GF Rag1^{-/-} mice (without bacterial reconstitution). Consequently, all GF Rag1^{-/-} mice succumbed to CDI within 2 days of infection. In contrast to growth of C. difficile in HC-HMA-WT mice, C. difficile was unable to grow in HC-HMA-Rag1^{-/-} mice (FIG. 2A). This result indicated that host T and B cell immunity is not required for the human microbiota to confer resistance against C. difficile. Rather, the host innate antimicrobial response, elicited by the microbiota, plays a role in the prevention of CDI. In this regard, data collected during the experiments indicated that colonization of GF Rag1^{-/-} mice with human microbiotas induced mucosal IL-22 expression (FIG. 2B). This evidence indicated that IL-22 is involved in microbiota-mediated protection against CDI. To test this hypothesis, experiments were conducted to examine blockade of IL-22 signaling in HC-HMA-Rag1^{-/-} mice on CDI susceptibility (FIG. 2C). IL-22 signaling was efficiently inhibited by an aIL-22 antibody in vivo. The antibody treatment also suppressed the expression of Reg3b and Reg3g, two antimicrobial proteins that are strongly induced in response to IL-22 (29) (FIG. 8). In this setting, colonization resistance of HC-HMA-Rag1^{-/-} mice was abolished and C. difficile was able to colonize the IL-22-neutralized HC-HMA-Rag1^{-/-} mice (FIG. 2C). In accordance with its colonization potential, C. difficile induced massive inflammation in HC-HMA-Rag1^{-/-} mice when IL-22 signaling was blocked (FIG. 2D). Thus, microbial induction of IL-22 reduces, minimizes, and/or eliminates C. difficile growth in HMA mice.

### Example 3—IL-22 Shapes the Gut Microbial Community and its Metabolic Function

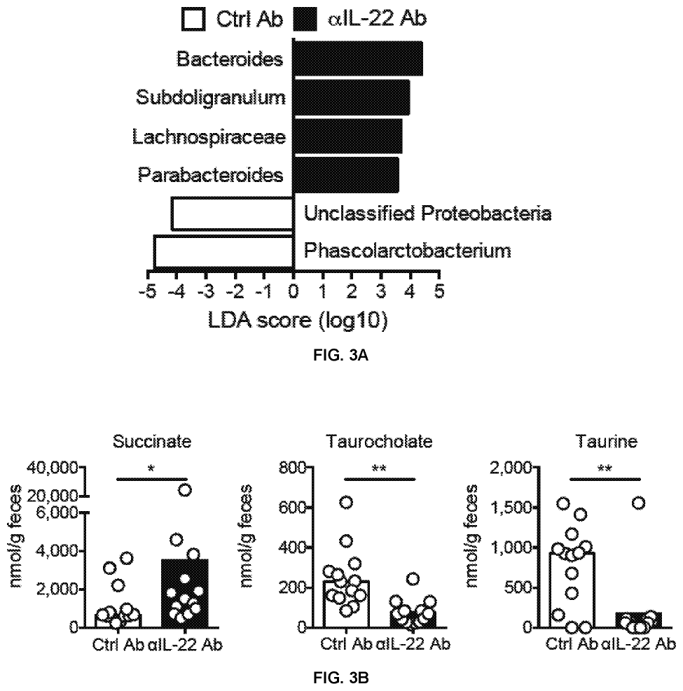

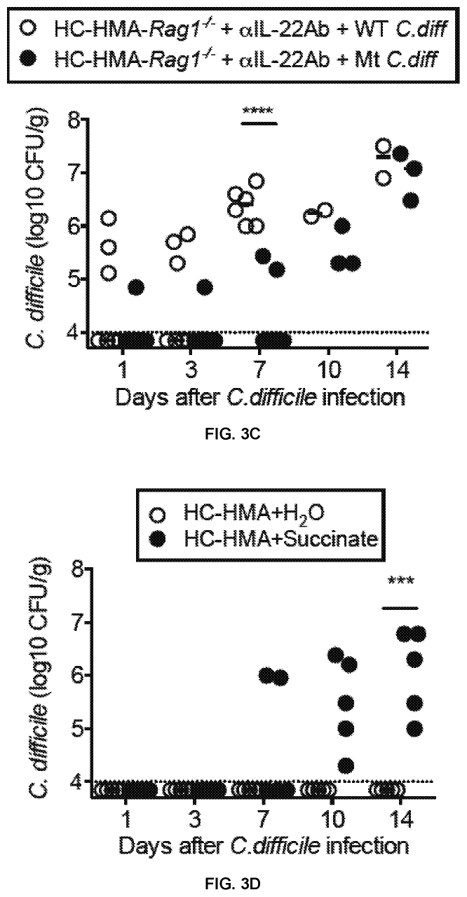

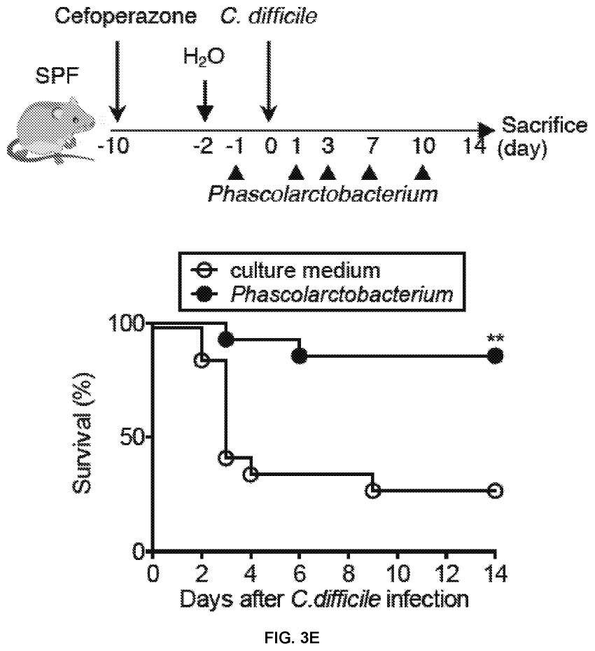

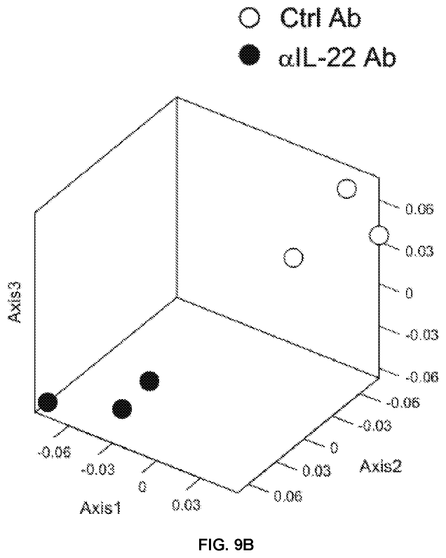

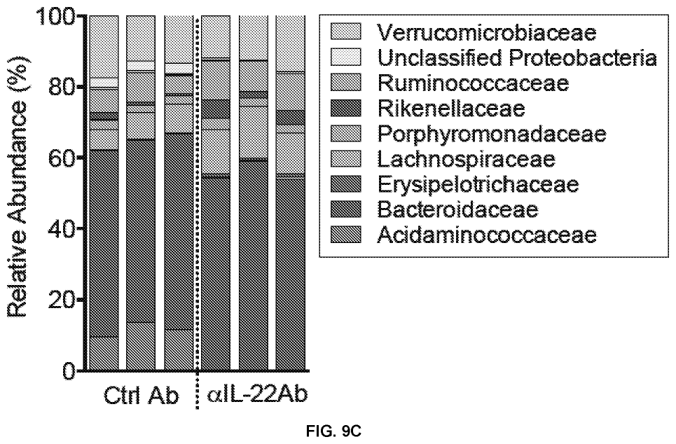

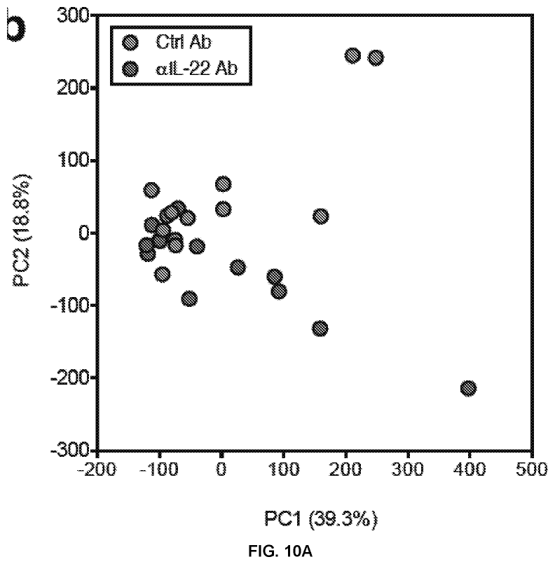

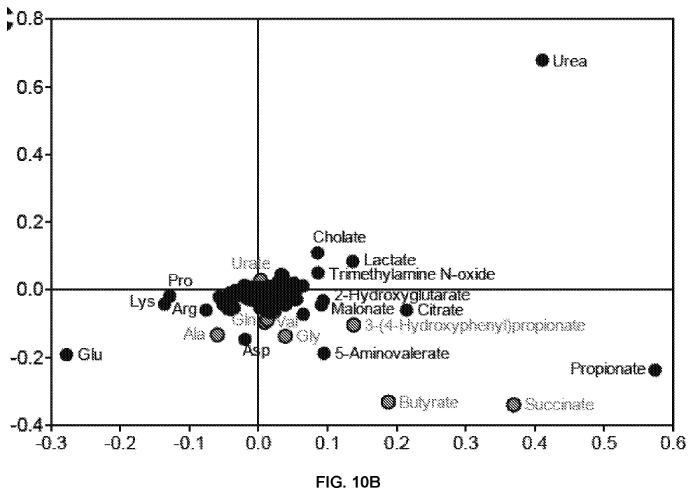

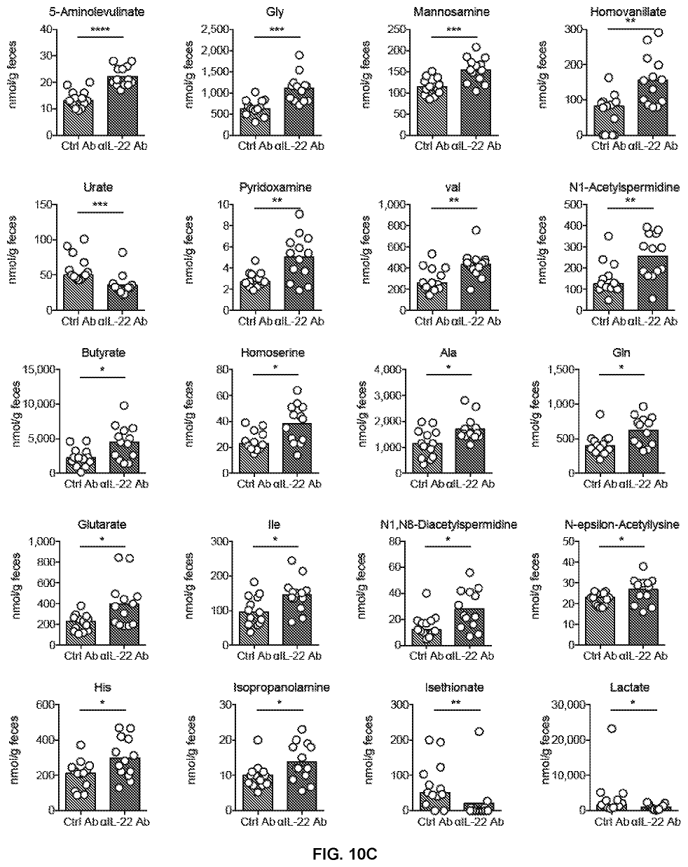

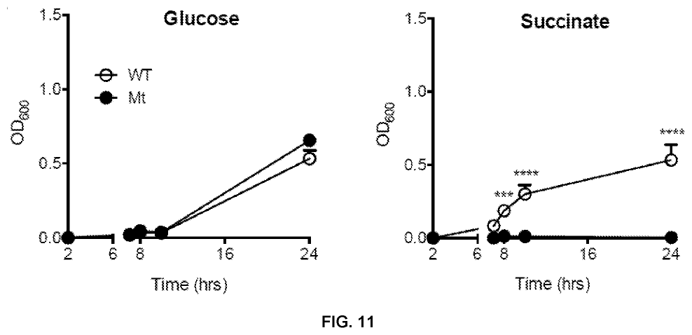

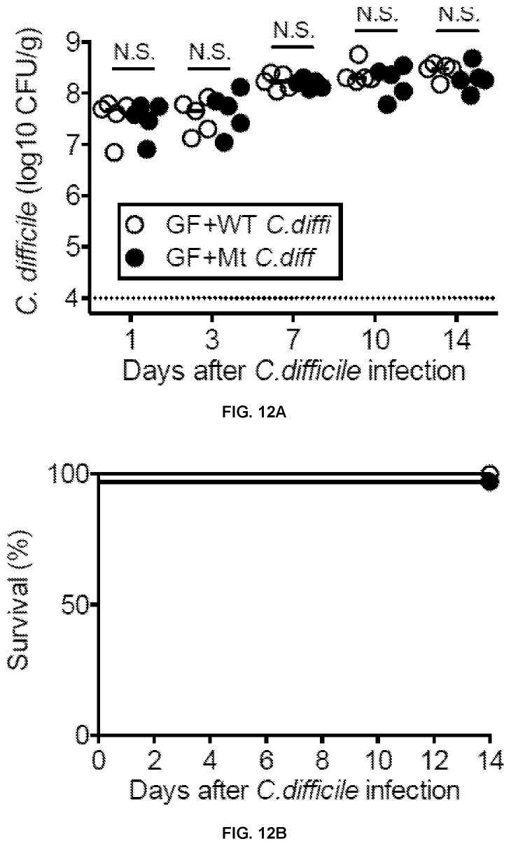

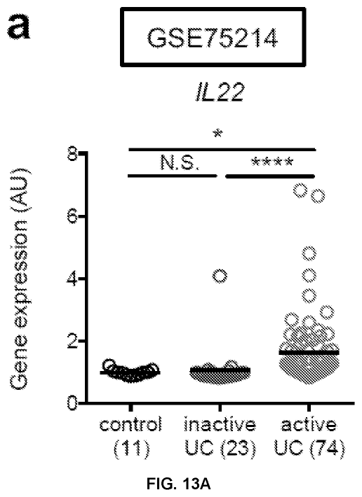

During the development of embodiments of the technology provided herein, experiments were conducted to test the mechanism by which IL-22 prevents CDI and examine the effect of IL-22 on the composition of the gut microbial community. The gut microbiome of HC-HMA-Rag1^{-/-} mice treated with the αIL-22 antibody was examined. IL-22 neutralization did not change the diversity and richness of the microbial communities (FIG. 9A). However, IL-22 neutralization did change the composition of the gut microbiota (FIG. 3A, FIG. 9B, and FIG. 9C). In particular, IL-22 neutralization produced a decrease in the abundance of organisms in the taxonomic family Acidaminococcaceae following IL-22 neutralization (FIG. 9C). LEfSe analysis further identified bacterial genera that became over-and under-represented following IL-22 neutralization (FIG. 3A). Among them, organisms in the genus Phascolarctobacterium (family Acidaminococcaceae) were significantly decreased as a result of IL-22 blockade (FIG. 3A). Phascolarctobacterium spp. are major consumers of succinate generated by other bacterial species, such as Bacteroides spp. (30, 31). Thus, the decreased abundance of Phascolarctobacterium may lead to abnormal succinate metabolism in the gut. Consistent with this hypothesis, luminal levels of succinate were significantly elevated in IL-22-neutralized HC-HMA-Rag1^{-/-} mice (FIG. 3B, FIG. 9A, FIG. 9B, FIG. 9C, and Table 1). Importantly, luminal succinate is known to aid the growth of C. difficile in the intestine (32). Hence, an imbalance in succinate metabolism due to gut dysbiosis, induced by a blockade of IL-22 signaling, may increase host susceptibility to CDI. Accordingly, experiments were conducted using a mutant strain of C. difficile that lacks the succinate utilization gene operon CD2344 (32). The CD2344 mutant of C. difficile (Cd-CD2344⁻), unlike its isogenic wild-type strain JIR8094, is unable to grow on succinate (32) (FIG. 11). IL-22-neutralized HC-HMA-Rag1^{-/-} mice, which are susceptible to CDI, were infected with either wild-type JIR8094 or Cd-CD2344⁻ (FIG. 3C). Wild-type C. difficile was able to colonize IL-22-neutralized HC-HMA-Rag1^{-/-} mice (FIG. 3C). In contrast, early colonization by Cd-CD2344-was markedly impaired (FIG. 3C). To address the extent to which succinate plays a role in the increased susceptibility to CDI, HC-HMA mice, which are protected against CDI, were given succinate. The data collected indicated that administration of succinate promoted the growth of C. difficile in HC-HMA mice (FIG. 3D), indicating that succinate is a metabolite that controls the colonization of C. difficile in the gut. Interestingly, Cd-CD2344⁻ was able to colonize in GF mice (FIG. 12), suggesting that C. difficile can employ alternate nutritional sources for its growth in the absence of commensal microbiota. Next, experiments were conducted to test if the colonization by succinate-consuming bacteria Phascolarctobacterium spp. can prevent CDI. SPF C57BL/6 mice were pre-treated with cefoperazone to decrease gut colonization resistance (9). As previously reported, cefoperazone-treated mice succumbed to CDI (FIG. 3E). Colonization by two strains of Phascolarctobacterium, e.g., P. faecium JCM30894 and P. succinatutens JCM16074, significantly improved mortality of C. difficile-infected mice (FIG. 3E). These results indicated that microbiota-induced IL-22 modulates (e.g., increases or decreases) the abundance of specific commensal bacteria, such as Phascolarctobacterium (e.g., which is increased by IL-22). Altered abundance of particular gut microbiota bacteria can lead to the accumulation of luminal metabolites, including succinate, which may foster the growth of C. difficile.

TABLE 1
Luminal metabolome changes in α-IL-22
antibody-treated HC-HMA-Rag1^{-/-} mice

| | Control | | Anti-IL-22 Ab | |
|---|---|---|---|---|
| COMPOUND (nmol/g) | Mean | SD | Mean | SD |
| Acetyl CoA | 31.10 | 14.91 | 34.72 | 18.91 |
| Adenine | 8.51 | 3.07 | 8.64 | 2.31 |
| Adenosine | 16.78 | 4.50 | 17.30 | 2.93 |
| Adipate | 11.72 | 15.39 | 18.03 | 20.04 |
| ADMA | 30.22 | 9.04 | 34.02 | 7.28 |
| ADP-ribose | 12.98 | 9.18 | 12.47 | 12.13 |
| Agmatine | 6.95 | 2.74 | 9.55 | 4.31 |
| Ala | 1148.67 | 541.40 | 1710.89 | 482.98 |
| Ala-Ala | 225.80 | 98.83 | 255.26 | 113.20 |
| alpha-Aminoadipate | 26.79 | 7.03 | 32.14 | 11.15 |
| Alpha-Methylserine | 6.26 | 3.38 | 8.35 | 4.37 |
| AMP | 66.23 | 11.87 | 74.30 | 14.80 |
| Arg | 511.77 | 233.23 | 706.08 | 260.93 |
| Argininosuccinate | 4.72 | 4.82 | 6.37 | 7.27 |
| Asn | 325.79 | 246.80 | 435.83 | 259.63 |
| Asp | 1510.13 | 903.79 | 1818.69 | 408.61 |
| Azelate | 73.67 | 12.02 | 85.48 | 36.70 |
| beta-Ala | 91.76 | 29.91 | 103.54 | 19.62 |
| Betaine | 86.79 | 187.91 | 68.62 | 45.64 |
| Betaine aldehyde | 39.78 | 25.79 | 34.97 | 32.34 |
| Butyrate | 2312.78 | 1338.16 | 4502.77 | 2497.82 |
| Cadaverine | 16.13 | 5.40 | 20.30 | 10.44 |
| Carnitine | 16.41 | 9.95 | 17.93 | 13.33 |
| Carnosine | 0.48 | 0.70 | 0.52 | 0.76 |
| Cholate | 411.81 | 529.47 | 235.75 | 180.21 |
| Choline | 140.90 | 140.10 | 83.91 | 25.74 |
| cis-Aconitate | 10.48 | 19.44 | 19.19 | 54.71 |
| Citraconate | 5.32 | 2.03 | 5.80 | 4.70 |
| Citramalate | 25.97 | 19.83 | 24.34 | 13.26 |
| Citrate | 455.77 | 779.13 | 654.53 | 1939.42 |
| Citrulline | 74.40 | 31.24 | 119.38 | 88.47 |
| CMP | 11.16 | 2.49 | 14.55 | 7.81 |
| Creatine | 8.64 | 8.80 | 24.87 | 47.09 |
| Creatinine | 57.54 | 107.08 | 53.25 | 136.43 |
| Cyclohexylamine | 26.17 | 46.09 | 21.98 | 34.55 |
| Cysteate | 57.88 | 34.39 | 36.55 | 40.16 |
| Cytidine | 25.01 | 14.93 | 23.07 | 12.21 |
| Cytosine | 3.25 | 1.81 | 4.72 | 3.45 |
| DCA | 23.98 | 83.99 | 46.06 | 31.03 |
| Diethanolamine | 27.76 | 33.68 | 19.03 | 14.31 |
| Dodecanedioate | 17.84 | 8.78 | 17.63 | 13.46 |
| dTMP | 18.96 | 4.07 | 18.56 | 6.20 |
| Ectoine | 8.83 | 6.59 | 9.44 | 5.47 |
| F6P | 28.13 | 18.53 | 29.93 | 18.22 |
| Fumarate | 11.36 | 16.21 | 21.03 | 32.00 |
| G1P | 27.87 | 17.42 | 26.43 | 13.22 |
| G6P | 130.41 | 90.47 | 132.27 | 68.35 |
| GABA | 36.94 | 15.89 | 70.32 | 67.54 |
| gamma-Butyrobetaine | 118.44 | 54.30 | 163.05 | 64.96 |
| gamma-Guanidinobutyrate | 17.23 | 15.57 | 14.42 | 16.23 |
| Gln | 422.74 | 159.01 | 624.34 | 211.03 |
| Glu | 7940.77 | 4059.08 | 9280.10 | 3345.28 |
| Glutarate | 216.70 | 80.78 | 397.10 | 227.00 |
| Gly | 650.71 | 189.55 | 1105.59 | 331.04 |
| Glycerate | 84.88 | 126.99 | 202.47 | 395.53 |
| Glycolate | 282.57 | 114.47 | 333.52 | 174.81 |
| Gly-Gly | 11.07 | 6.99 | 9.83 | 6.90 |
| Guanidinoacetate | 23.21 | 52.16 | 27.68 | 86.23 |
| Guanidinosuccinate | 6.89 | 7.23 | 5.86 | 4.88 |
| Guanine | 40.22 | 16.22 | 38.05 | 10.40 |
| Guanosine | 16.29 | 18.01 | 18.33 | 18.72 |
| His | 205.06 | 80.20 | 298.33 | 113.63 |
| Histamine | 15.14 | 8.94 | 16.99 | 9.11 |
| Homoserine | 25.12 | 7.54 | 38.29 | 14.94 |
| Homovanillate | 65.32 | 52.05 | 155.83 | 71.04 |
| Hydroxyproline | 123.64 | 57.24 | 170.10 | 91.93 |
| Hypoxanthine | 43.25 | 47.81 | 42.12 | 53.47 |
| Ile | 99.76 | 43.47 | 145.95 | 48.11 |
| Imidazole-4-acetate | 8.41 | 9.58 | 8.56 | 10.54 |
| Indole-3-ethanol | 34.79 | 20.90 | 37.14 | 22.98 |
| Inosine | 213.14 | 154.23 | 193.63 | 148.36 |
| Isethionate | 73.29 | 65.16 | 21.19 | 61.54 |
| Isoamylamine | 0.85 | 0.76 | 0.73 | 0.75 |
| Isocitrate | 17.03 | 38.21 | 19.72 | 59.78 |
| Isopropanolamine | 9.92 | 3.56 | 13.90 | 5.45 |
| Lactate | 3764.70 | 6028.05 | 1020.51 | 748.85 |
| Leu | 163.13 | 89.48 | 232.41 | 101.14 |
| Lys | 1834.55 | 585.25 | 2452.85 | 1238.26 |
| Malate | 130.04 | 55.96 | 163.28 | 100.64 |
| Malonate | 299.34 | 205.73 | 445.19 | 321.48 |
| Mannosamine | 114.50 | 19.81 | 154.70 | 29.60 |
| Met | 161.47 | 147.00 | 167.92 | 40.08 |
| Methionine sulfoxide | 62.57 | 51.59 | 60.83 | 30.67 |
| N,N-Dimethylglycine | 4.27 | 7.83 | 24.63 | 32.02 |
| N1,N12-Diacetylspermine | 3.60 | 2.66 | 4.91 | 3.25 |
| N1,N8-Diacetylspermidine | 15.30 | 9.02 | 27.90 | 15.32 |
| N1-Acetylspermidine | 150.42 | 78.64 | 256.61 | 105.69 |
| N6,N6,N6-Trimethyllysine | 17.90 | 21.03 | 14.39 | 5.58 |
| N8-Acetylspermidine | 10.04 | 9.19 | 15.34 | 11.56 |
| N-Acetylaspartate | 138.33 | 101.25 | 228.13 | 174.40 |
| N-Acetylglucosamine | 129.26 | 72.47 | 125.79 | 51.39 |
| N-Acetylglucosamine | 34.49 | 7.13 | 36.34 | 17.02 |
| 1-phosphate | | | | |
| N-Acetylglucosamine | 42.98 | 20.83 | 33.20 | 11.73 |
| 6-phosphate | | | | |
| N-Acetylglutamate | 166.74 | 74.76 | 270.87 | 145.51 |
| N-Acetylhistidine | 7.55 | 6.82 | 10.32 | 12.38 |
| N-Acetylornithine | 22.63 | 9.67 | 33.40 | 18.44 |
| N-Acetylputrescine | 77.33 | 112.18 | 22.98 | 15.26 |
| N-Acetylvaline | 13.28 | 31.05 | 18.31 | 56.21 |
| NAD+ | 377.40 | 179.43 | 491.89 | 206.96 |
| NADP+ | 20.20 | 9.61 | 23.94 | 10.47 |
| N-alpha, N-alpha- | 0.21 | 0.76 | 1.49 | 2.38 |
| Dimethylhistidine | | | | |
| N-epsilon-Acetyllysine | 22.17 | 2.94 | 26.93 | 6.29 |
| Nicotinamide | 47.53 | 15.49 | 42.25 | 8.29 |
| Nicotinate | 150.93 | 72.55 | 193.96 | 105.55 |
| N-Methylalanine | 2.98 | 1.81 | 3.67 | 2.89 |
| N-Methylglutamate | 0.44 | 1.59 | 1.76 | 2.35 |
| O-Acetylserine | 19.37 | 9.84 | 37.56 | 37.12 |
| O-Phosphoserine | 12.34 | 5.58 | 11.60 | 5.40 |
| Ophthalmate | 2.68 | 6.48 | 17.21 | 18.58 |
| Ornithine | 121.83 | 53.23 | 275.43 | 293.95 |
| p-Aminobenzoate | 1.53 | 2.07 | 3.26 | 3.03 |
| Pentanoate | 144.39 | 67.18 | 222.25 | 147.66 |
| Phe | 80.40 | 33.26 | 111.85 | 45.36 |
| Phenaceturate | 32.75 | 79.14 | 22.84 | 74.59 |
| Phthalate | 4.40 | 1.43 | 4.26 | 1.33 |
| Pimelate | 20.25 | 6.77 | 24.83 | 8.02 |
| Pipecolate | 50.50 | 31.28 | 75.27 | 50.15 |
| Pro | 1024.01 | 426.68 | 1117.94 | 594.66 |
| Proline betaine | 24.70 | 39.66 | 21.31 | 51.39 |
| Propionate | 9745.21 | 6008.69 | 12287.33 | 9213.76 |
| Putrescine | 36.16 | 47.07 | 10.30 | 3.75 |
| (1,4-Butanediamine) | | | | |
| Pyridoxamine | 2.90 | 0.72 | 5.01 | 2.15 |
| Pyridoxamine 5′-phosphate | 11.87 | 7.95 | 15.87 | 8.22 |
| Ru5P | 28.22 | 23.31 | 25.24 | 15.36 |
| S7P | 48.56 | 10.90 | 47.05 | 28.73 |
| Saccharopine | 16.36 | 11.85 | 21.29 | 17.88 |
| SAM+ | 30.38 | 11.53 | 39.66 | 13.69 |
| Sarcosine | 75.74 | 38.66 | 118.09 | 112.24 |
| SDMA | 10.34 | 5.53 | 10.69 | 4.59 |
| Sebacate | 9.80 | 2.98 | 10.16 | 5.24 |
| Ser | 439.75 | 196.64 | 459.07 | 144.61 |
| Serotonin | 10.59 | 5.37 | 6.95 | 3.87 |
| Spermidine | 112.73 | 39.13 | 133.95 | 53.83 |
| Succinate | 1166.87 | 1092.11 | 3527.79 | 6362.90 |
| Taurine | 781.19 | 502.87 | 172.13 | 419.01 |
| Taurocholate | 248.67 | 147.14 | 76.53 | 62.78 |
| Thiamine | 9.11 | 3.33 | 13.00 | 5.92 |
| Thr | 341.84 | 119.72 | 467.28 | 191.58 |
| Thymidine | 244.46 | 114.07 | 223.42 | 141.52 |
| Thymine | 164.35 | 158.16 | 133.48 | 61.57 |
| Trigonelline | 16.79 | 29.63 | 12.32 | 26.56 |
| Trimethylamine N-oxide | 152.63 | 245.63 | 133.29 | 173.79 |
| Trp | 60.85 | 23.30 | 78.11 | 25.27 |
| Tyr | 201.77 | 113.09 | 287.23 | 122.63 |
| Tyramine | 6.88 | 9.89 | 4.82 | 3.74 |
| UDP | 18.29 | 4.45 | 22.63 | 9.26 |
| UMP | 39.11 | 11.93 | 38.22 | 16.24 |
| Uracil | 66.05 | 73.26 | 120.93 | 138.52 |
| Urate | 60.10 | 19.27 | 35.78 | 15.57 |
| Urea | 5086.32 | 11950.14 | 958.59 | 2065.22 |
| Uridine | 173.07 | 56.84 | 169.72 | 87.28 |
| Urocanate | 10.05 | 4.34 | 11.34 | 7.62 |
| Val | 293.39 | 114.25 | 432.15 | 128.14 |
| Xanthine | 51.00 | 74.33 | 74.41 | 97.88 |
| Xanthurenate | 12.36 | 10.84 | 12.70 | 17.28 |
| 1-Methylhistamine | 0.88 | 1.39 | 0.33 | 0.54 |
| 1-Methylnicotinamide | 0.90 | 1.43 | 1.70 | 2.95 |
| 1,3-Diaminopropane | 9.06 | 16.48 | 9.04 | 14.42 |
| 2,5-Dihydroxybenzoate | 25.84 | 7.48 | 28.87 | 15.88 |
| 2-Hydroxybutyrate | 6.21 | 11.18 | 15.58 | 13.70 |
| 2-Hydroxyglutarate | 216.34 | 130.58 | 254.26 | 369.06 |
| 2-Isopropylmalate | 4.07 | 3.08 | 7.39 | 8.55 |
| 2-Oxoglutarate | 42.16 | 57.02 | 32.30 | 43.00 |
| 3-(4-Hydroxyphenyl) | 642.05 | 404.38 | 941.66 | 760.16 |
| propionate | | | | |
| 3-Aminoisobutyrate | 13.75 | 8.90 | 14.94 | 11.20 |
| 3-Indoxyl sulfate | 27.81 | 66.52 | 8.55 | 24.47 |
| 3-Methylbutanoate | 150.01 | 63.53 | 178.68 | 66.84 |
| 3-Methylhistidine | 9.06 | 5.32 | 9.71 | 8.11 |
| 3PG | 40.83 | 41.64 | 30.83 | 15.22 |
| 3-Ureidopropionate | 24.95 | 61.21 | 34.44 | 112.90 |
| 4-(beta- | 10.09 | 2.38 | 13.82 | 5.37 |
| Acetylaminoethyl)imidazole | | | | |
| 4-Acetylbutyrate | 8.25 | 6.69 | 6.64 | 6.05 |
| 4-Hydroxymethylimidazole | 5.75 | 1.96 | 5.65 | 1.33 |
| 5-Aminolevulinate | 13.80 | 3.18 | 22.11 | 3.76 |
| 5-Aminovalerate | 2306.87 | 515.51 | 2916.19 | 892.28 |
| 5-Hydroxyindoleacetate | 859.02 | 72.91 | 991.89 | 262.52 |
| 5-Hydroxylysine | 19.29 | 21.09 | 15.70 | 4.27 |
| 5-Methyl-2′-deoxycytidine | 21.69 | 13.79 | 14.02 | 15.91 |
| 5-Methylcytosine | 0.95 | 1.32 | 0.36 | 0.90 |
| 5-Methylthioadenosine | 9.81 | 4.01 | 11.12 | 3.00 |
| 5-Oxoproline | 69.73 | 31.87 | 64.06 | 15.37 |
| 7-Methylguanine | 20.68 | 15.14 | 15.63 | 13.79 |

Values reported are mean concentrations and standard deviations in nmol/g from 13 independent experiments for the control and 13 independent experiments for the anti-IL-22 antibody.

### Example 4— Host Mucus Glycosylation regulates Gut Microbiota

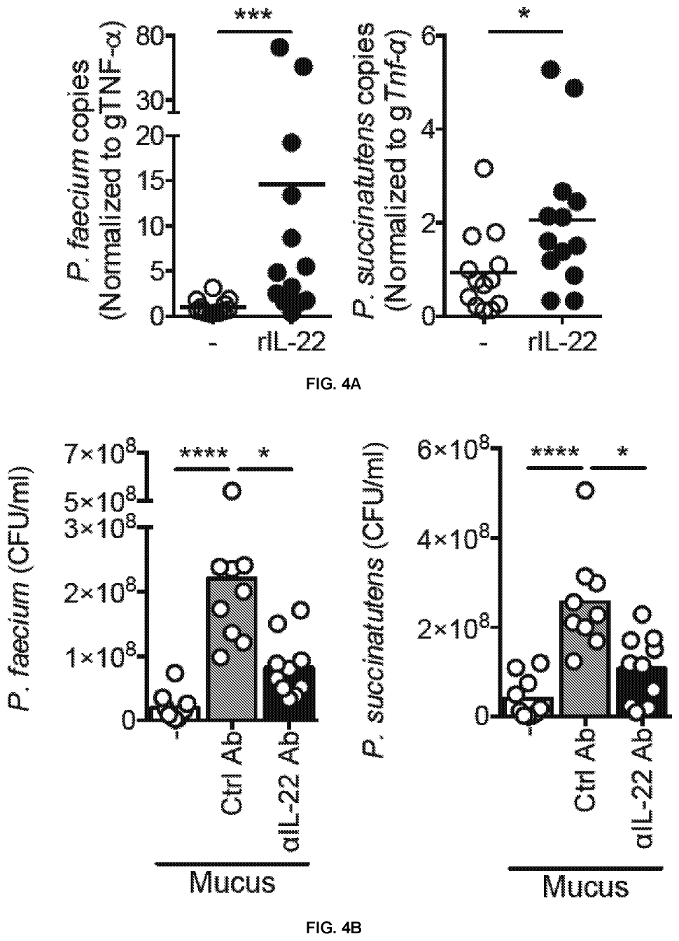

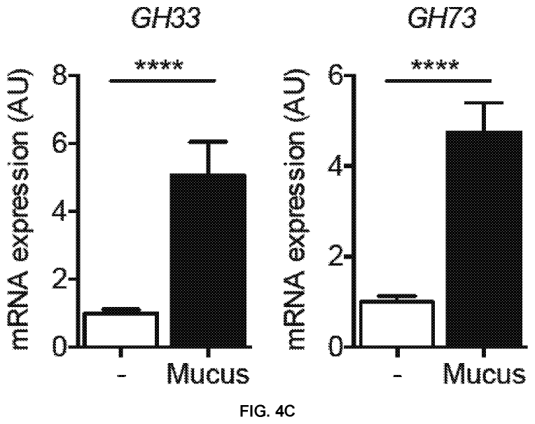

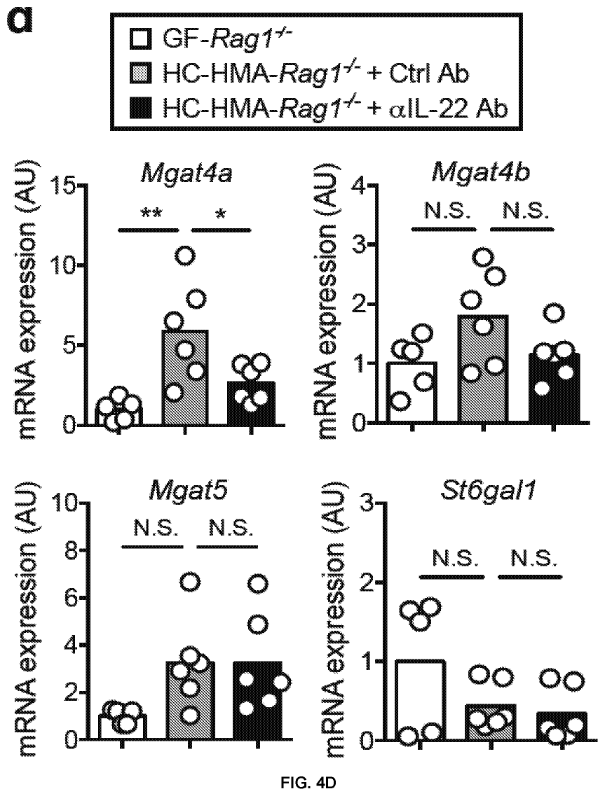

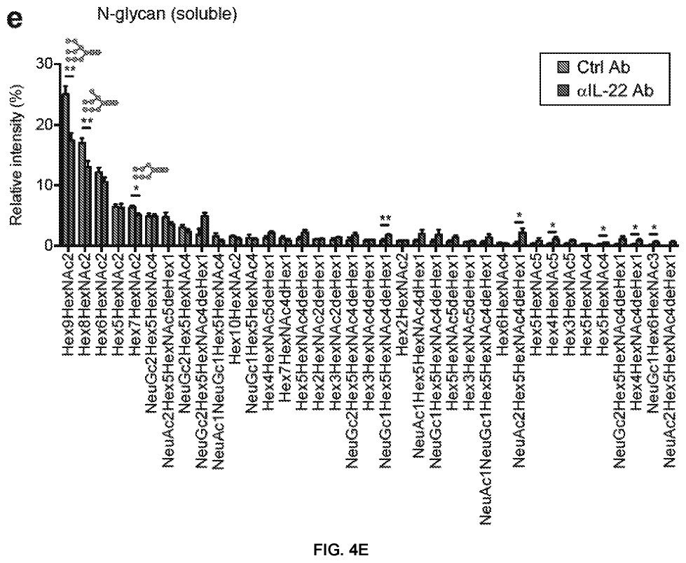

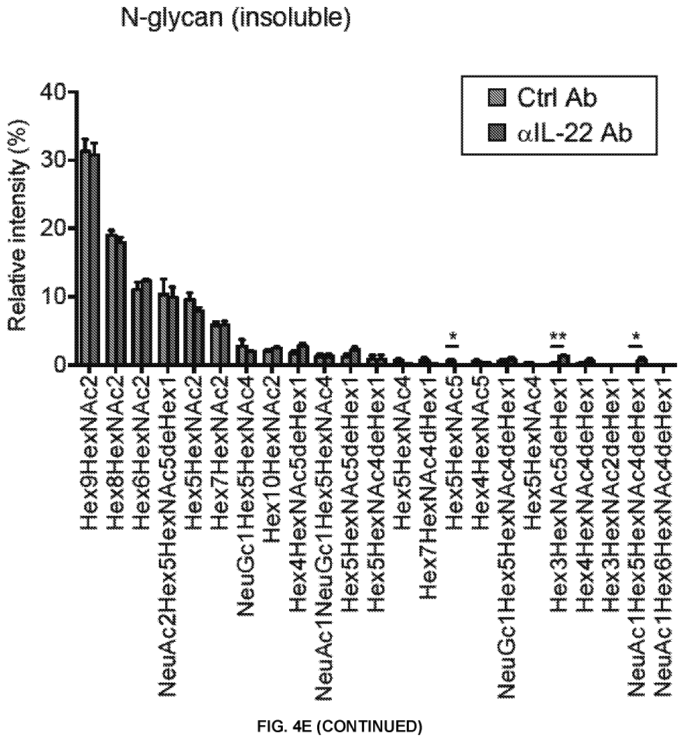

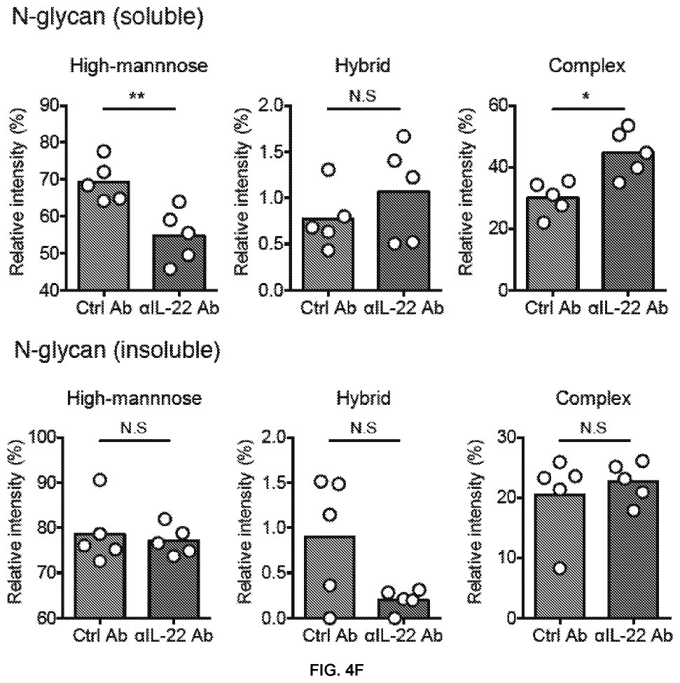

Defects in IL-22-mediated regulation of microbial metabolic activity can result in the accumulation of luminal succinate. Given that the abundance of Phascolarctobacterium in the gut significantly decreased when IL-22 signaling was blocked, it was conceivable that IL-22 directly promotes the growth of Phascolarctobacterium. To address this possibility, GF mice were colonized with two strains of Phascolarctobacterium, P. faecium JCM30894 and P. succinatutens JCM16074, followed by treatment with an IL-22-Fc fusion protein (FIG. 4A). The gut colonization efficiency of both strains was significantly enhanced by IL-22-Fc, suggesting that IL-22 directly regulates the growth of Phascolarctobacterium (FIG. 4A). The substrate usability of Phascolarctobacterium is limited; in particular, growth of Phascolarctobacterium is primarily limited by the availability of succinate and carbohydrates and/or SCFAs do not appear to be of major importance (30). However, succinate is not available in GF mice since succinate-producing commensal bacteria are absent (32). This indicates that Phascolarctobacterium can utilize other nutritional sources for its growth in gnotobiotic mice treated with IL-22-Fc. Data collected indicated that P. succinatutens JCM16074 harbors enzymes that belong to the glycoside hydrolase (GH) families GH73 and GH33 (GenBank/EMBL/DDBJ accession AB490811). Given that both GH73 and GH33 are host glycan-related enzyme families (e.g., related to N-acetylglucosamine (GlcNAc) and Sialic acid, respectively), it is plausible that Phascolarctobacterium is adapted to consume host-derived glycans in the gut. Therefore, experiments were conducted to test whether IL-22 regulates host glycosylation in the gut, which in turn promotes the growth of Phascolarctobacterium by increasing the availability of usable host glycans. In particular, colonic mucus was isolated from SPF mice treated with the αIL-22 antibody or the isotype control antibody. Isolated mucus was added to the Phascolarctobacterium growth medium in vitro. Supplementation with mucus isolated from control mice promoted the growth of P. faecium and P. succinatutens (FIG. 4B). In contrast, colonic mucus isolated from IL-22-neutralized mice did not efficiently promote the growth of P. faecium and P. succinatutens (FIG. 4B). P. succinatutens expressed GH33 and GH73 when cultured with colonic mucus, indicating that P. succinatutens employs host mucus glycan for its growth (FIG. 4C). These results suggested that IL-22-induced host mucus glycosylation likely promotes the growth of Phascolarctobacterium. Expression of host glycosyltransferases in HC-HMA mice was measured and data were collected to determine if these enzymes are regulated by IL-22 signaling. As shown in FIG. 4D, host glycosyltransferases related to N-glycosylation processing, such as Mgat4a, were induced in GF mice upon colonization with a human microbiota. The expression levels of these enzymes were significantly reduced when IL-22 signaling was blocked (FIG. 4d), indicating that the microbiota-induced IL-22 was responsible for the induction of these enzymes. Next, experiments were conducted to analyze the host glycosylation status of HMA-Rag-1^{-/-} mice with or without IL-22 signal blockade. Colonic mucosal scrapes from HMA-Rag-1^{-/-} mice treated with the αIL-22 antibody or the isotype control antibody were isolated and N-glycan profiles in soluble and insoluble fractions were analyzed (FIG. 4E). The data indicated that N-glycosylation of the host proteins was impaired in IL-22-neutralized mice both in the insoluble fraction (contains mainly glycosylated mucus proteins, such as Muc2) and soluble fractions (contains epithelial surface glycans) (FIG. 4F). Notably, high-mannose type glycans were reduced in the soluble fraction and hybrid type glycans were generally decreased in the insoluble fraction in IL-22-neutralized mice (FIG. 4F). These data suggest that IL-22-mediated host N-glycosylation regulates the growth of Phascolarctobacterium in the gut.

### Example 5—Impaired Host N-glycosylation in UC Patients

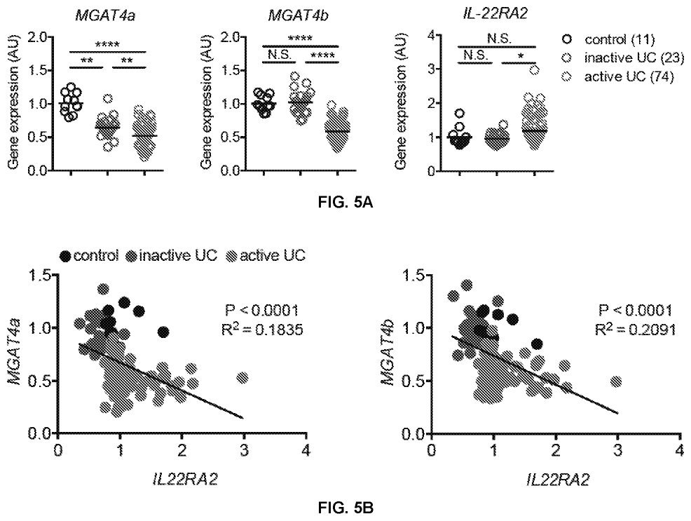

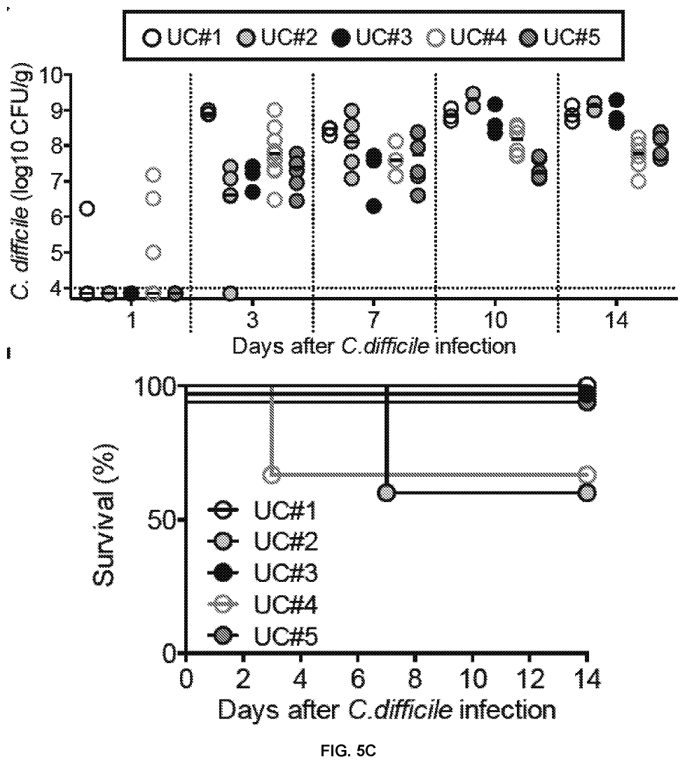

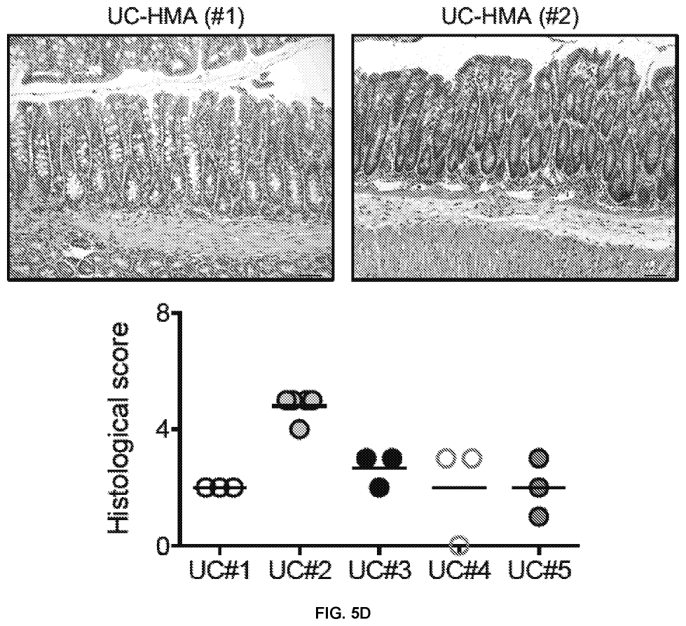

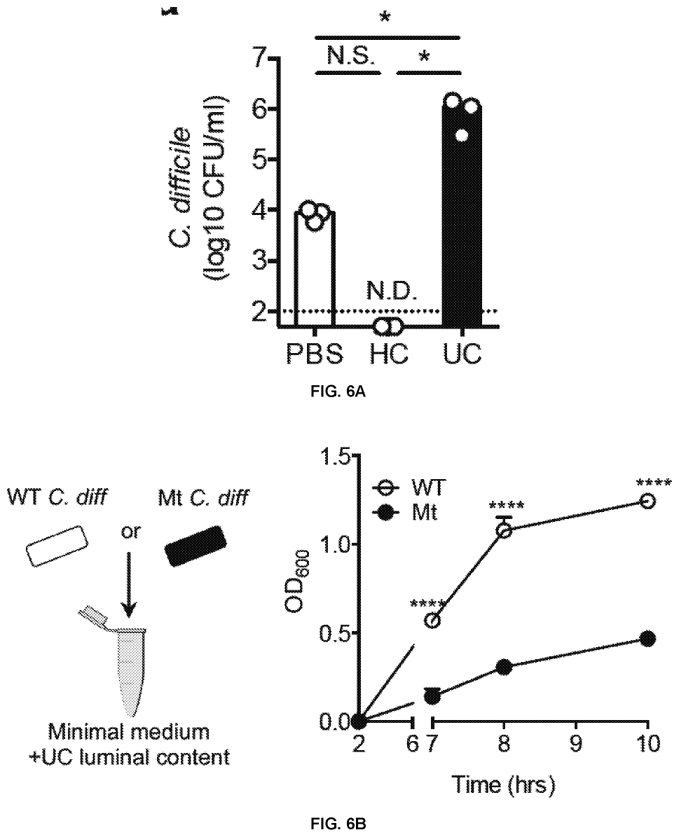

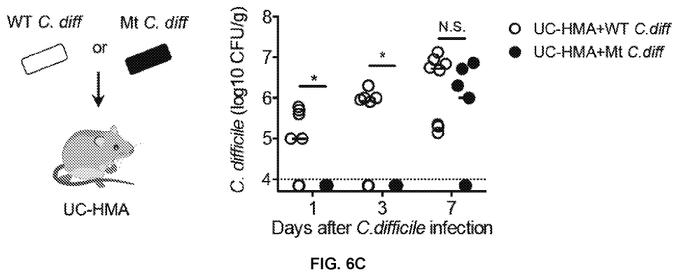

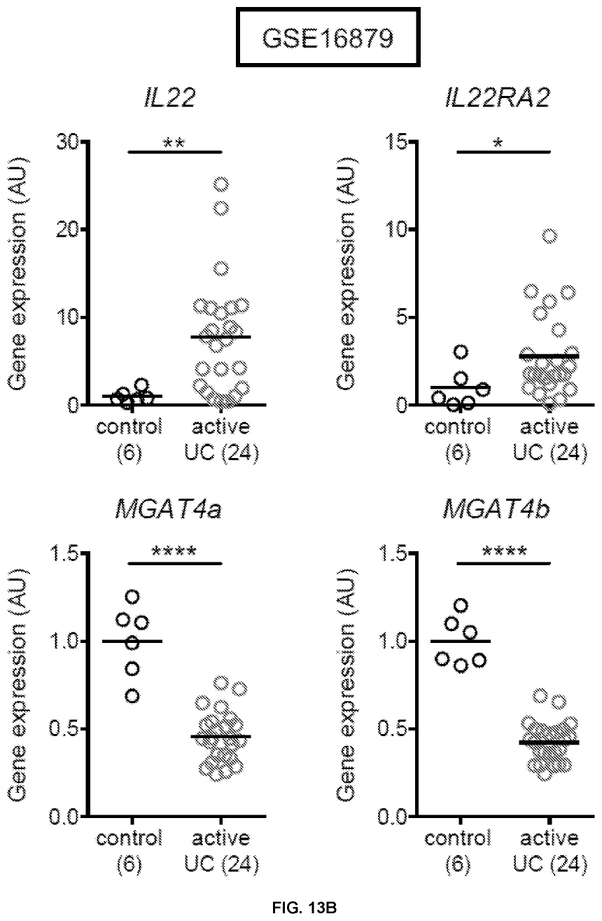

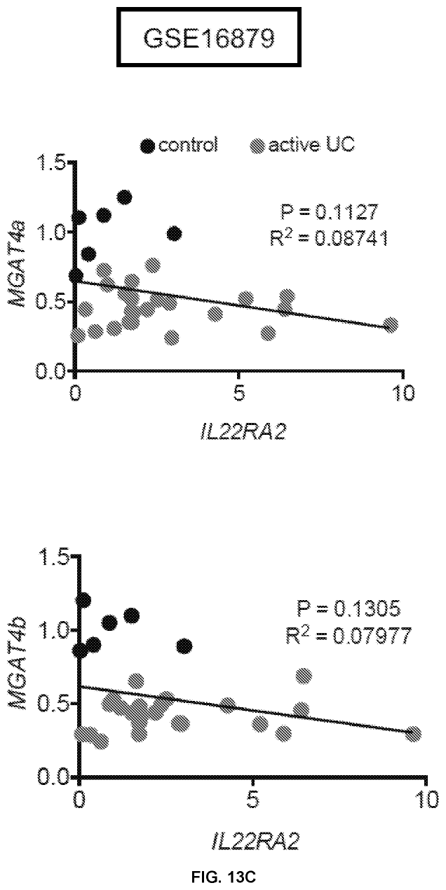

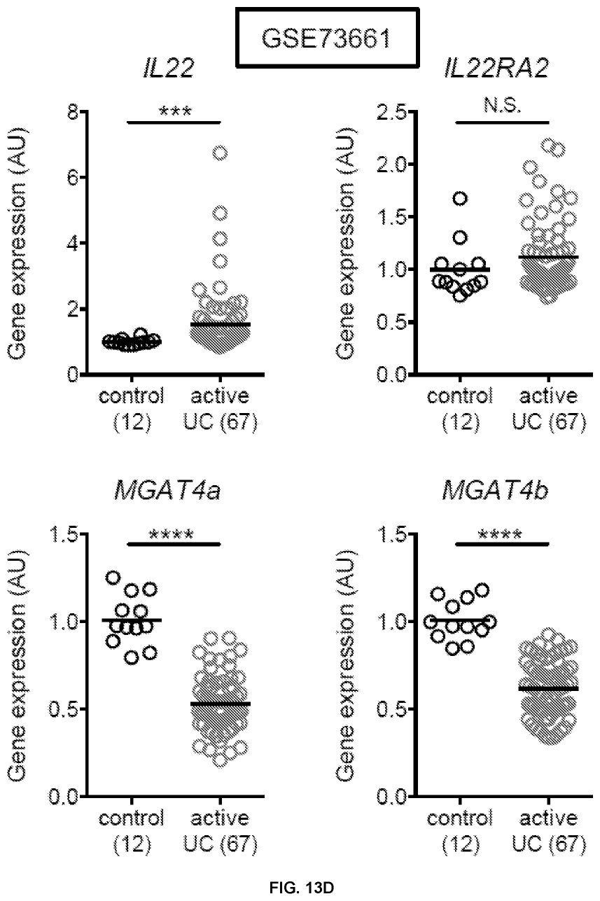

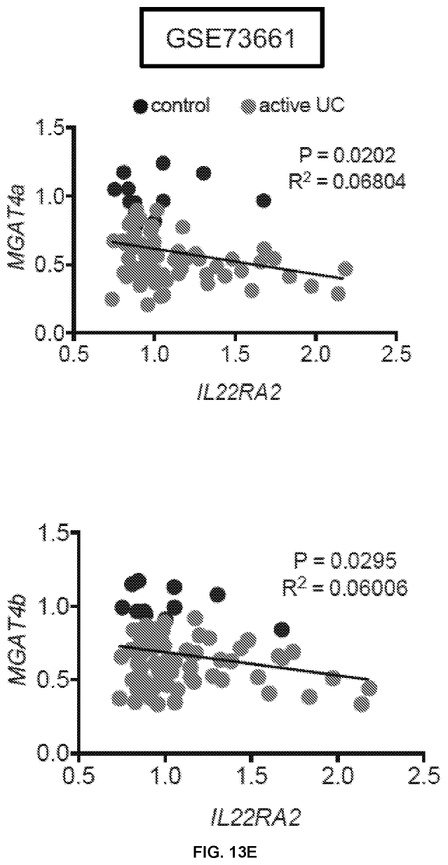

During the development of embodiments of the technology provided herein, experiments were conducted to examine IL-22-mediated host glycosylation and increased host susceptibility to CDI resulting from underlying disease. In particular, patients with inflammatory bowel disease (IBD) were studied because patients with IBD, particularly ulcerative colitis (UC), are at higher risk for CDI compared to non-IBD individuals (33, 34, 35). UC patients have perturbed microbial communities even in the absence of antibiotics, a condition termed dysbiosis (36). Given the importance of the resident microbiota in the prevention of CDI, it is plausible that gut dysbiosis in UC renders patients susceptible to CDI. Accordingly, IL-22-mediated host glycosylation and the risk of CDI in UC patients were evaluated. Analysis of a public database (GSE75214) (37) indicated that the expression of host glycosyltransferases, such as MGAT4A and MGAT4B, was significantly impaired in UC patients compared to normal controls (FIG. 5A). The expression of IL22RA2 (also known as IL-22 binding protein (BP)), a soluble receptor for IL-22 that antagonizes IL-22-IL-22R signaling, was markedly up-regulated in patients with UC, while IL-22 expression was not affected (FIG. 5A and FIG. 13A). Notably, IL22RA2 expression was inversely correlated with the expression of these glycosyltransferases, indicating that impaired IL-22 signaling, resulting from the overexpression of IL-22BP, may lead to decreased host glycosylation in UC (FIG. 5B). An analysis of two other deposited cohorts (GSE16879 38 and GSE73661 39) revealed similar expression patterns and inverse correlation between IL22RA2 and host glycosylation enzymes (FIG. 13B to 13E). Consistent with these results, HMA mice colonized with the microbiotas derived from UC patients (UC-HMA mice) were unable to resist C. difficile colonization and some mice succumbed to infection (FIG. 5C and 5D). To address whether impaired colonization resistance is caused by the altered metabolic activity of the gut microbiota in UC, an ex vivo C. difficile growth assay was performed using luminal contents isolated from UC-HMA mice. Consistent with the in vivo data, inoculated C. difficile failed to proliferate in luminal contents isolated from HC-HMA mice (FIG. 6A). On the other hand, C. difficile grew in luminal contents isolated from UC-HMA mice (FIG. 6A). As expected, the growth of C. difficile was dependent on succinate—the Cd-CD2344⁻ mutant exhibited impaired growth in UC luminal contents in vitro and in vivo (FIG. 6B and 6C).

### Example 6—Restoration of Microbial Metabolism reduces the Risk for CDI

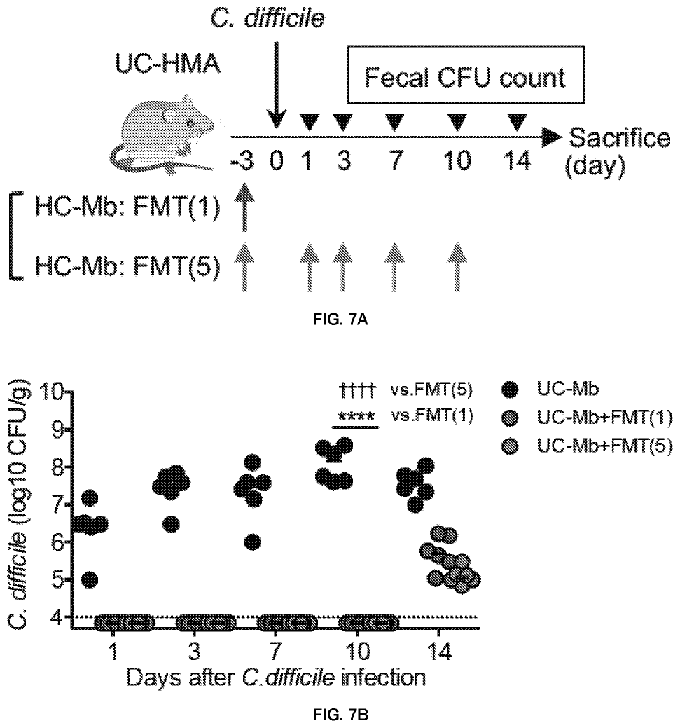

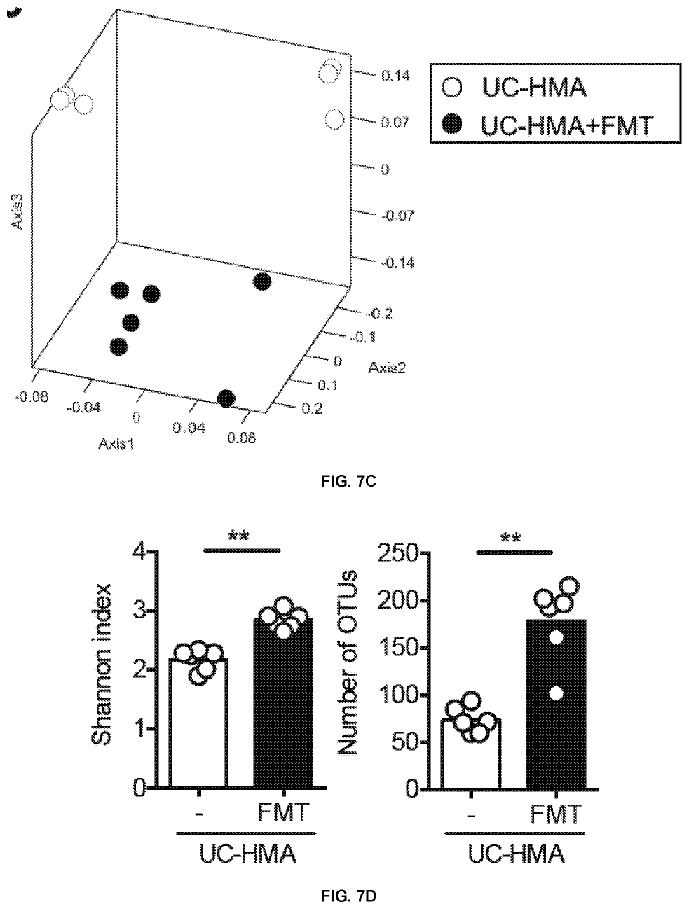

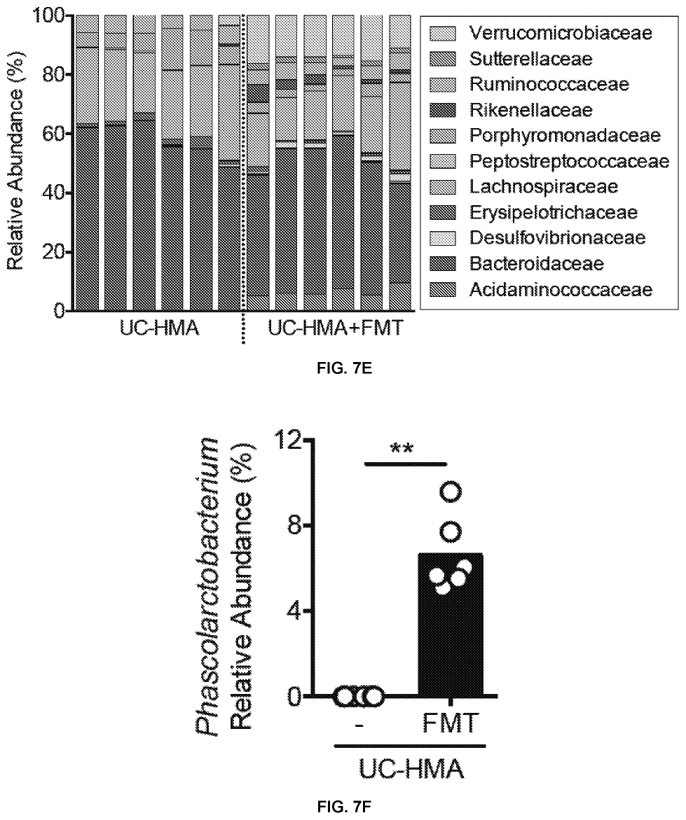

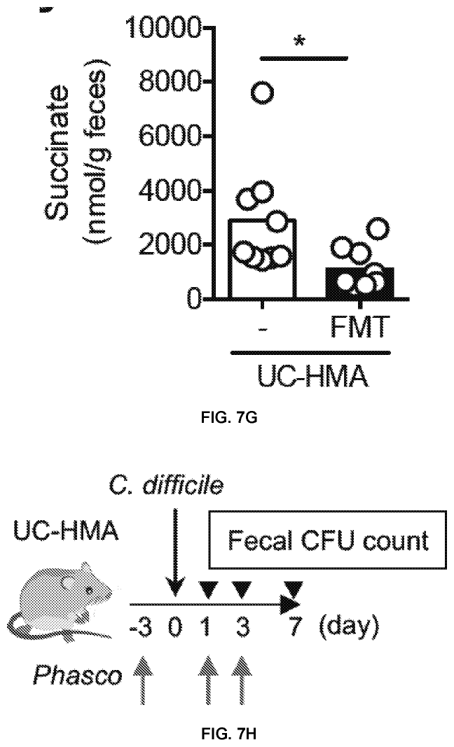

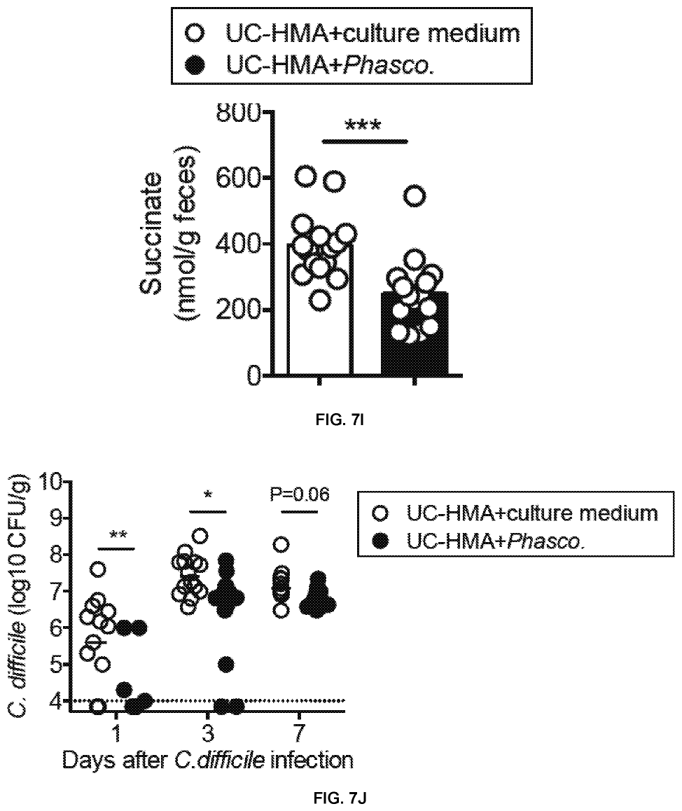

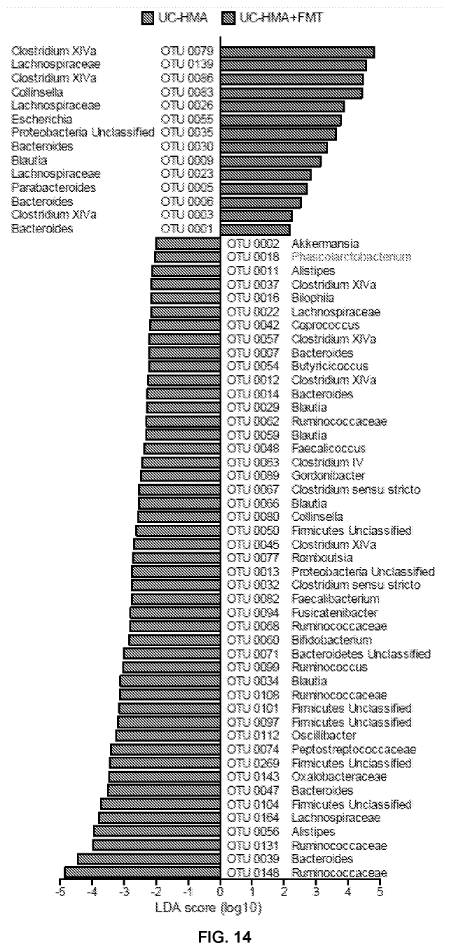

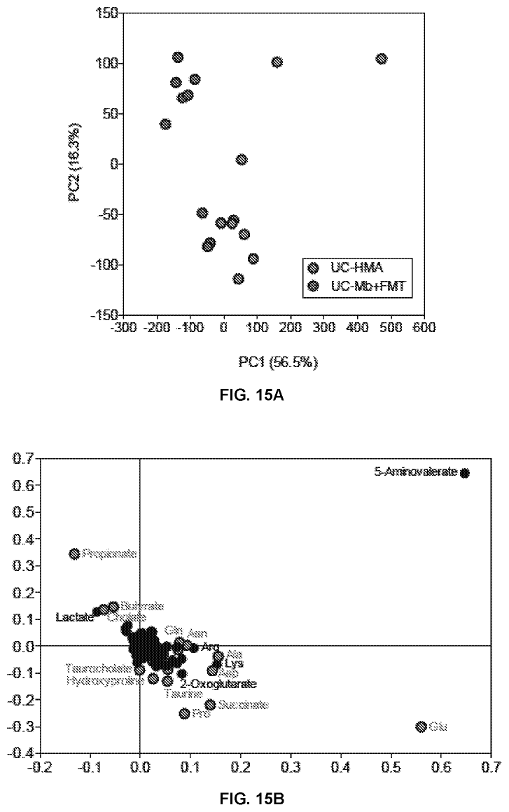

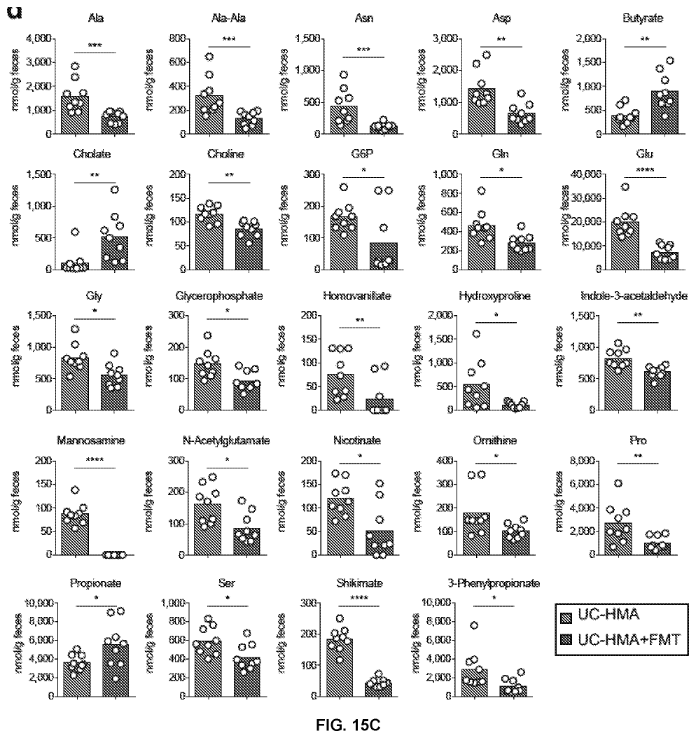

During the development of embodiments of the technology provided herein, experiments were conducted to reduce the risk of CDI in UC-HMA mice by restoring the metabolic activity of the gut microbiota. In particular, FMT was used to restore normal microbial metabolic activity and colonization resistance in UC-HMA mice. Healthy microbiotas isolated from HC-HMA mice were inoculated into UC-HMA mice, which were then infected with C. difficile (FIG. 7A). As a result, FMT almost completely prevented the growth of C. difficile in the gut (FIG. 7B). The data indicated that a single FMT was equally effective at preventing CDI as multiple FMTs (FIG. 7B). Gut dysbiosis seen in UC-HMA mice (e.g., decreased diversity and richness) improved significantly as a result of FMT (FIGS. 7C to 7E). More specifically, FMT restored the abundance of Phascolarctobacterium (FIG. 7F and FIG. 14). Consistent with the restoration of Phascolarctobacterium abundance, the levels of luminal succinate were significantly reduced following FMT (FIG. 7G, FIG. 15, and Table 2). To validate the importance of Phascolarctobacterium in the prevention of CDI, UC-HMA mice were inoculated with succinate consumers, P. faecium and P. succinatutens, instead of a full FMT (FIG. 7H and FIG. 16). As expected, colonization of P. faecium and P. succinatutens significantly reduced the concentration of luminal succinate (FIG. 7I and FIG. 16). After reducing luminal succinate availability, inoculation of Phascolarctobacterium significantly inhibited the colonization of C. difficile, particularly in early stages of infection (FIG. 7J and FIG. 16). Thus, the accumulation of luminal succinate (e.g., caused by gut dysbiosis) causes increased susceptibility of UC patients to CDI.

TABLE 2
Luminal metabolome changes in FMT-treated UC-HMA mice

| | UC-HMA | | UC-HMA + FMT | |
|---|---|---|---|---|
| COMPOUND (nmol/g) | Mean | SD | Mean | SD |
| Acetyl COA | 29.14 | 9.45 | 13.22 | 4.65 |
| Adenine | 4.78 | 2.37 | 5.58 | 1.20 |
| Adenosine | 16.26 | 4.14 | 12.84 | 5.50 |
| ADMA | 23.85 | 9.34 | 19.18 | 9.90 |
| ADP | 41.62 | 15.04 | 21.76 | 13.24 |
| Agmatine | 7.09 | 3.40 | 4.52 | 2.18 |
| Ala | 1586.28 | 659.98 | 730.80 | 229.18 |
| Ala-Ala | 321.79 | 161.44 | 132.75 | 55.16 |
| Allantoin | 230.49 | 109.32 | 153.78 | 162.69 |
| alpha-Aminoadipate | 29.63 | 3.96 | 25.05 | 4.65 |
| Alpha-Methylserine | 15.18 | 8.77 | 10.43 | 6.36 |
| AMP | 76.43 | 30.00 | 71.96 | 48.42 |
| Arg | 660.78 | 407.84 | 405.70 | 205.95 |
| Asn | 435.58 | 270.97 | 122.40 | 49.42 |
| Asp | 1442.09 | 549.96 | 648.87 | 305.40 |
| Azelate | 73.04 | 17.51 | 77.94 | 11.56 |
| beta-Ala | 78.37 | 66.50 | 57.72 | 39.99 |
| beta-Imidazolelactate | 44.69 | 23.52 | 21.80 | 32.71 |
| Betaine | 73.60 | 23.19 | 86.07 | 40.88 |
| Betonicine | 1.70 | 5.11 | 2.01 | 4.19 |
| Butyrate | 391.82 | 173.50 | 897.01 | 378.50 |
| Cadaverine | 16.78 | 6.88 | 16.74 | 3.12 |
| Carnitine | 23.56 | 8.94 | 56.68 | 20.64 |
| Cholate | 111.29 | 185.89 | 523.62 | 375.04 |
| Choline | 116.89 | 16.48 | 85.72 | 15.74 |
| cis-Aconitate | 0.80 | 2.40 | 0.00 | 0.00 |
| Citraconate | 3.09 | 3.27 | 2.58 | 2.55 |
| Citramalate | 20.22 | 15.94 | 17.88 | 7.31 |
| Citrate | 191.53 | 176.60 | 88.02 | 55.53 |
| Citrulline | 54.84 | 9.55 | 127.57 | 84.23 |
| CMP | 12.48 | 4.56 | 16.55 | 8.58 |
| Creatine | 28.58 | 26.78 | 152.91 | 242.46 |
| Creatinine | 11.42 | 4.68 | 14.75 | 6.93 |
| Cyclohexylamine | 0.53 | 1.05 | 0.63 | 1.26 |
| Cysteate | 41.31 | 8.46 | 21.06 | 21.91 |
| Cysteine S-sulfate | 0.00 | 0.00 | 2.39 | 3.61 |
| Cytidine | 4.67 | 5.60 | 6.62 | 10.64 |
| Cytosine | 6.13 | 9.32 | 2.40 | 2.86 |
| DCA | 19.64 | 30.14 | 113.99 | 92.20 |
| Decanoate | 0.00 | 0.00 | 5.75 | 4.45 |
| Diethanolamine | 40.15 | 36.60 | 36.51 | 26.14 |
| Dodecanedioate | 4.19 | 3.71 | 25.38 | 12.09 |
| dTMP | 19.02 | 3.53 | 28.97 | 8.40 |
| Ectoine | 5.02 | 0.91 | 3.98 | 0.72 |
| F6P | 35.11 | 3.68 | 18.44 | 21.29 |
| FAD | 15.00 | 8.69 | 14.91 | 8.40 |
| Fumarate | 16.17 | 25.90 | 2.20 | 6.59 |
| G1P | 27.34 | 7.20 | 11.08 | 14.37 |
| G6P | 166.18 | 43.95 | 85.78 | 99.22 |
| GABA | 85.96 | 33.86 | 69.91 | 23.43 |
| gamma-Butyrobetaine | 175.38 | 68.23 | 154.98 | 80.25 |
| gamma-Glu-2AB | 118.71 | 25.41 | 0.00 | 0.00 |
| gamma-Guanidinobutyrate | 27.71 | 13.33 | 8.06 | 2.26 |
| Gln | 467.97 | 159.88 | 283.66 | 84.43 |
| Glu | 19907.35 | 6361.71 | 7441.63 | 3024.10 |
| Glucosaminate | 7.98 | 5.44 | 1.35 | 4.06 |
| Glucosamine | 25.81 | 30.86 | 45.75 | 17.83 |
| Glutarate | 42.20 | 29.90 | 45.67 | 18.21 |
| Gly | 835.70 | 217.26 | 555.96 | 178.55 |
| Glycerate | 96.91 | 80.38 | 103.94 | 82.51 |
| Glycerophosphate | 146.31 | 43.74 | 93.66 | 30.96 |
| Glycerophosphorylcholine | 32.74 | 14.56 | 0.00 | 0.00 |
| Glycolate | 250.73 | 110.09 | 319.85 | 60.16 |
| Gly-Gly | 24.53 | 1.93 | 14.88 | 3.95 |
| Gly-Leu | 49.82 | 8.16 | 32.92 | 5.43 |
| Guanidinoacetate | 4.45 | 2.49 | 3.09 | 4.58 |
| Guanidinosuccinate | 3.71 | 5.59 | 0.00 | 0.00 |
| Guanine | 62.13 | 14.25 | 71.79 | 21.20 |
| Guanosine | 0.00 | 0.00 | 5.03 | 10.35 |
| Heptanoate | 0.00 | 0.00 | 0.91 | 2.72 |
| Hexanoate | 0.00 | 0.00 | 11.64 | 7.55 |
| His | 182.39 | 75.64 | 162.24 | 72.79 |
| Histamine | 11.63 | 5.27 | 10.19 | 2.21 |
| Homoserine | 43.44 | 10.54 | 35.54 | 4.87 |
| Homovanillate | 76.62 | 46.46 | 23.30 | 39.20 |
| Hydroxyproline | 547.19 | 513.88 | 107.42 | 63.58 |
| Hypoxanthine | 14.66 | 8.05 | 22.98 | 24.60 |
| Ile | 90.55 | 35.79 | 95.78 | 79.87 |
| Indole-3-acetaldehyde | 824.93 | 145.10 | 607.32 | 89.49 |
| Indole-3-acetamide | 85.35 | 14.97 | 63.91 | 14.20 |
| Inosine | 54.73 | 53.26 | 130.38 | 181.10 |
| Isethionate | 32.66 | 5.14 | 20.47 | 25.59 |
| Isocitrate | 12.66 | 5.42 | 0.00 | 0.00 |
| Isopropanolamine | 91.51 | 68.72 | 17.97 | 38.05 |
| Lactate | 1164.95 | 328.99 | 1813.45 | 1048.00 |
| Leu | 199.62 | 111.17 | 209.67 | 187.25 |
| Lys | 2622.72 | 1082.00 | 2179.96 | 838.21 |
| Malate | 209.46 | 170.54 | 88.83 | 87.20 |
| Malonate | 244.62 | 92.76 | 49.92 | 78.31 |
| Mannosamine | 87.18 | 22.80 | 0.00 | 0.00 |
| Met | 161.39 | 45.92 | 118.64 | 45.38 |
| Methionine sulfoxide | 72.78 | 11.02 | 84.51 | 37.10 |
| N1,N12-Diacetylspermine | 1.46 | 1.72 | 1.06 | 0.95 |
| N1,N8-Diacetylspermidine | 62.93 | 32.09 | 36.94 | 37.44 |
| N1-Acetylspermidine | 88.02 | 44.89 | 51.85 | 51.17 |
| N6,N6,N6-Trimethyllysine | 11.55 | 3.87 | 26.14 | 17.91 |
| N8-Acetylspermidine | 10.85 | 5.12 | 4.30 | 3.00 |
| N-Acetylaspartate | 77.31 | 25.49 | 93.29 | 39.86 |
| N-Acetylglucosamine | 85.13 | 70.18 | 14.61 | 43.84 |
| N-Acetylglucosamine | 49.60 | 19.13 | 33.26 | 16.84 |
| 1-phosphate | | | | |
| N-Acetylglucosamine | 66.39 | 17.37 | 28.95 | 13.16 |
| 6-phosphate | | | | |
| N-Acetylglucosylamine | 7.95 | 23.85 | 0.00 | 0.00 |
| N-Acetylglutamate | 160.78 | 59.66 | 86.50 | 47.18 |
| N-Acetylmethionine | 17.47 | 12.09 | 9.50 | 2.87 |
| N-Acetylneuraminate | 28.60 | 19.40 | 15.75 | 11.25 |
| N-Acetylornithine | 19.89 | 4.93 | 13.50 | 3.78 |
| N-Acetylputrescine | 192.58 | 57.05 | 111.93 | 143.33 |
| NAD+ | 560.99 | 264.23 | 349.28 | 169.88 |
| NADP+ | 29.10 | 11.51 | 13.80 | 3.69 |
| N-epsilon-Acetyllysine | 28.42 | 9.22 | 20.81 | 6.17 |
| Nicotinamide | 49.08 | 10.01 | 66.26 | 21.80 |
| Nicotinate | 120.49 | 35.81 | 51.20 | 55.38 |
| N-Methylalanine | 0.00 | 0.00 | 4.15 | 3.19 |
| O-Acetylcarnitine | 0.00 | 0.00 | 0.68 | 1.43 |
| O-Acetylserine | 12.03 | 14.60 | 3.95 | 7.90 |
| Octanoate | 0.00 | 0.00 | 6.43 | 5.18 |
| O-Phosphoserine | 41.46 | 23.41 | 10.66 | 3.95 |
| Ophthalmate | 63.94 | 15.68 | 0.00 | 0.00 |
| Ornithine | 178.61 | 95.40 | 101.64 | 29.13 |
| p-Aminobenzoate | 0.00 | 0.00 | 1.38 | 2.74 |
| Pantothenate | 2.96 | 4.60 | 8.46 | 5.80 |
| Pelargonate | 13.02 | 5.17 | 19.99 | 9.74 |
| Pentanoate | 0.00 | 0.00 | 39.58 | 118.74 |
| Phe | 89.61 | 40.09 | 73.70 | 35.73 |
| Phosphorylcholine | 110.30 | 40.27 | 45.36 | 38.39 |
| Phthalate | 2.69 | 0.66 | 2.93 | 0.66 |
| p-Hydroxyphenylacetate | 29.29 | 59.41 | 68.73 | 114.11 |
| Pimelate | 11.62 | 4.46 | 11.48 | 6.44 |
| Pipecolate | 60.16 | 13.65 | 64.83 | 10.57 |
| Pro | 2732.19 | 1654.47 | 1050.53 | 557.91 |
| Proline betaine | 11.32 | 6.93 | 8.36 | 4.93 |
| Propionate | 3607.76 | 870.07 | 5541.85 | 2432.88 |
| Putrescine(1,4-Butanediamine) | 35.48 | 11.79 | 32.06 | 19.12 |
| Pyridoxal | 14.59 | 3.67 | 14.59 | 2.19 |
| Pyridoxamine | 0.30 | 0.90 | 0.36 | 1.07 |
| Pyridoxamine 5′-phosphate | 6.95 | 9.17 | 4.46 | 6.88 |
| Ru5P | 25.68 | 16.44 | 21.10 | 35.30 |
| S7P | 54.59 | 9.07 | 23.51 | 24.19 |
| Saccharopine | 31.30 | 10.26 | 2.80 | 5.57 |
| SAM+ | 31.57 | 13.94 | 29.76 | 31.04 |
| Sarcosine | 74.61 | 14.14 | 100.54 | 35.27 |
| SDMA | 14.18 | 3.66 | 13.64 | 5.38 |
| Sebacate | 4.93 | 1.92 | 7.00 | 1.31 |
| Ser | 595.03 | 147.40 | 419.15 | 137.83 |
| Serotonin | 9.32 | 5.49 | 11.56 | 5.60 |
| Shikimate | 183.50 | 37.59 | 43.31 | 13.26 |
| Spermidine | 124.58 | 105.46 | 71.45 | 57.37 |
| Succinate | 2889.32 | 2025.06 | 1110.58 | 767.32 |
| Synephrine | 2.90 | 8.71 | 0.00 | 0.00 |
| Taurine | 1436.13 | 692.49 | 913.73 | 961.05 |
| Taurocholate | 215.82 | 123.27 | 292.23 | 339.96 |
| Thiamine | 8.72 | 3.86 | 26.18 | 10.65 |
| Thr | 321.97 | 114.13 | 218.81 | 123.29 |
| Threonate | 172.64 | 46.78 | 239.08 | 124.63 |
| Thymidine | 76.39 | 33.51 | 65.06 | 39.67 |
| Thymine | 145.75 | 21.48 | 127.03 | 26.36 |
| trans-4-Hydroxy-3- | 38.11 | 13.23 | 20.31 | 14.46 |
| methoxycinnamate | | | | |
| Trigonelline | 5.22 | 2.19 | 1.09 | 3.26 |
| Trp | 75.86 | 35.03 | 57.82 | 24.71 |
| Tryptamine | 2.02 | 2.45 | 0.00 | 0.00 |
| Tyr | 239.27 | 138.13 | 178.38 | 81.28 |
| Tyramine | 16.27 | 8.59 | 9.38 | 3.72 |
| UDP | 34.68 | 12.11 | 20.86 | 10.68 |
| UDP-glucose | 22.14 | 12.61 | 12.33 | 9.01 |
| UDP-glucuronate | 6.20 | 9.59 | 0.00 | 0.00 |
| UDP-N-acetylglucosamine | 53.41 | 35.36 | 33.24 | 11.36 |
| UMP | 48.13 | 25.06 | 52.36 | 40.84 |
| Uracil | 0.00 | 0.00 | 12.62 | 25.05 |
| Urate | 54.72 | 9.50 | 65.68 | 17.93 |
| Urea | 10.96 | 22.22 | 8.42 | 16.84 |
| Uridine | 199.04 | 93.48 | 157.66 | 103.84 |
| Urocanate | 15.19 | 4.39 | 16.01 | 11.28 |
| Val | 298.15 | 126.64 | 329.82 | 235.08 |
| Xanthine | 0.00 | 0.00 | 43.34 | 87.17 |
| Xanthurenate | 0.99 | 2.98 | 0.00 | 0.00 |
| 1-Methylnicotinamide | 21.07 | 8.75 | 0.57 | 1.71 |
| 1,3-Diaminopropane | 0.00 | 0.00 | 1.95 | 4.02 |
| 2,5-Dihydroxybenzoate | 16.74 | 7.17 | 21.42 | 11.59 |
| 2AB | 112.17 | 58.57 | 124.48 | 43.38 |
| 2-Hydroxybutyrate | 0.00 | 0.00 | 4.75 | 9.43 |
| 2-Hydroxyglutarate | 357.95 | 206.65 | 72.27 | 62.50 |
| 2-Hydroxypentanoate | 81.99 | 32.57 | 41.85 | 71.28 |
| 2-Isopropylmalate | 2.73 | 1.21 | 0.68 | 1.40 |
| 2-Oxoglutarate | 580.33 | 337.45 | 297.75 | 457.51 |
| 2-Oxoisopentanoate | 0.00 | 0.00 | 7.82 | 15.92 |
| 3-Aminoisobutyrate | 105.58 | 27.40 | 18.50 | 24.77 |
| 3-Aminopropane-1,2-diol | 36.29 | 11.34 | 23.57 | 13.16 |
| 3-Hydroxyanthranilate | 27.24 | 45.76 | 15.90 | 23.79 |
| 3-Hydroxybutyrate | 64.71 | 38.70 | 66.06 | 16.85 |
| 3-Methylbutanoate | 156.89 | 49.24 | 203.95 | 120.14 |
| 3-Methylhistidine | 7.49 | 1.56 | 6.88 | 2.73 |
| 3PG | 68.30 | 29.47 | 29.40 | 13.01 |
| 3-Phenyllactate | 130.71 | 37.67 | 148.06 | 56.54 |
| 3-Phenylpropionate | 348.27 | 67.99 | 282.59 | 56.07 |
| 4-(beta-Acetylaminoethyl) | 7.04 | 4.13 | 8.11 | 2.88 |
| imidazole | | | | |
| 4-Hydroxymethylimidazole | 6.32 | 1.35 | 7.67 | 1.49 |
| 4-Oxopentanoate | 12.85 | 15.39 | 21.40 | 13.62 |
| 4-Pyridoxate | 8.32 | 3.52 | 10.18 | 2.61 |
| 5-Aminolevulinate | 9.08 | 6.45 | 11.79 | 2.38 |
| 5-Aminovalerate | 16429.85 | 15427.03 | 5218.52 | 2796.60 |
| 5-Hydroxyindoleacetate | 1021.96 | 260.18 | 873.95 | 164.31 |
| 5-Hydroxylysine | 82.80 | 24.80 | 44.57 | 51.39 |
| 5-Methylthioadenosine | 10.40 | 3.71 | 12.74 | 10.57 |
| 5-Oxoproline | 739.12 | 516.62 | 593.49 | 399.11 |
| 6-Aminohexanoate | 0.00 | 0.00 | 9.38 | 7.82 |
| 6-Hydroxynicotinate | 43.01 | 12.58 | 0.00 | 0.00 |
| 7-Methylguanine | 21.23 | 3.97 | 5.27 | 8.00 |

Values reported are mean concentrations and standard deviations in nmol/g from 9 independent experiments for the UC-HMA and 9 independent experiments for the UC-HMA+FMT.

### REFERENCES

1. Britton, R. A. & Young, V. B. Role of the intestinal microbiota in resistance to colonization by Clostridium difficile. Gastroenterology 146, 1547-1553 (2014).
2. Smits, W. K., Lyras, D., Lacy, D. B., Wilcox, M. H. & Kuijper, E. J. Clostridium difficile infection. Nature reviews. Disease primers 2, 16020 (2016).
3. Kamada, N., Chen, G. Y., Inohara, N. & Nunez, G. Control of pathogens and pathobionts by the gut microbiota. Nat Immunol 14, 685-690 (2013).
4. Seekatz, A. M. & Young, V. B. Clostridium difficile and the microbiota. J Clin Invest 124, 4182-4189 (2014).
5. Kamada, N. & Nunez, G. Role of the gut microbiota in the development and function of lymphoid cells. J Immunol 190, 1389-1395 (2013).
6. Koropatkin, N. M., Cameron, E. A. & Martens, E. C. How glycan metabolism shapes the human gut microbiota. Nat Rev Microbiol 10, 323-335 (2012).
7. Buffie, C. G. et al. Precision microbiome reconstitution restores bile acid mediated resistance to Clostridium difficile. Nature 517, 205-208 (2015).
8. Sorg, J. A. & Sonenshein, A. L. Bile salts and glycine as cogerminants for Clostridium difficile spores. J Bacteriol 190, 2505-2512 (2008).
9. Theriot, C. M. et al. Antibiotic-induced shifts in the mouse gut microbiome and metabolome increase susceptibility to Clostridium difficile infection. Nat Commun 5, 3114 (2014).
10. Wilson, K. H., Kennedy, M. J. & Fekety, F. R. Use of sodium taurocholate to enhance spore recovery on a medium selective for Clostridium difficile. J Clin Microbiol 15, 443-446 (1982).
11. Sonnenberg, G. F., Monticelli, L. A., Elloso, M. M., Fouser, L. A. & Artis, D. CD4(+) lymphoid tissue-inducer cells promote innate immunity in the gut. Immunity 34, 122-134 (2011).
12. Zenewicz, L. A. & Flavell, R .A. Recent advances in IL-22 biology. Int Immunol 23, 159-163 (2011).
13. Guo, X. et al. Induction of innate lymphoid cell-derived interleukin-22 by the transcription factor STAT3 mediates protection against intestinal infection. Immunity 40, 25-39 (2014).
14. Sonnenberg, G. F. et al. Innate lymphoid cells promote anatomical containment of lymphoid-resident commensal bacteria. Science 336, 1321-1325 (2012).
15. Zheng, Y. et al. Interleukin-22 mediates early host defense against attaching and effacing bacterial pathogens. Nat Med 14, 282-289 (2008).
16. Sakamoto, K. et al. IL-22 Controls Iron-Dependent Nutritional Immunity Against Systemic Bacterial Infections. Science immunology 2 (2017).
17. Pham, T. A. et al. Epithelial IL-22RA1-mediated fucosylation promotes intestinal colonization resistance to an opportunistic pathogen. Cell Host Microbe 16, 504-516 (2014).
18. Pickard, J. M. et al. Rapid fucosylation of intestinal epithelium sustains host-commensal symbiosis in sickness. Nature 514, 638-641 (2014).
19. Satoh-Takayama, N. et al. Microbial flora drives interleukin 22 production in intestinal NKp46+cells that provide innate mucosal immune defense. Immunity 29, 958-970 (2008).
20. Sanos, S. L. et al. RORgammat and commensal microflora are required for the differentiation of mucosal interleukin 22-producing NKp46+ cells. Nat Immunol 10, 83-91 (2009).
21. Crellin, N. K. et al. Regulation of cytokine secretion in human CD127(+) LTi-like innate lymphoid cells by Toll-like receptor 2. Immunity 33, 752-764 (2010).
22. Zelante, T. et al. Tryptophan Catabolites from Microbiota Engage Aryl Hydrocarbon Receptor and Balance Mucosal Reactivity via Interleukin-22. Immunity 39, 372-385 (2013).
23. Sadighi Akha, A. A. et al. Interleukin-22 and CD160 play additive roles in the host mucosal response to Clostridium difficile infection in mice. Immunology 144, 587-597 (2015).
24. Hasegawa, M. et al. Interleukin-22 regulates the complement system to promote resistance against pathobionts after pathogen-induced intestinal damage. Immunity 41, 620-632 (2014).
25. Abt, M. C. et al. Innate Immune Defenses Mediated by Two ILC Subsets Are Critical for Protection against Acute Clostridium difficile Infection. Cell Host Microbe 18, 27-37 (2015).
26. Nagao-Kitamoto, H. et al. Functional characterization of inflammatory bowel disease-associated gut dydbiosis in gnotobiotic mice. Cellular and Molecular Gastroenterology and Hepatology 2, 468-481 (2016).
27. Ridaura, V. K. et al. Gut microbiota from twins discordant for obesity modulate metabolism in mice. Science 341, 1241214 (2013).
28. Collins, J., Auchtung, J. M., Schaefer, L., Eaton, K. A. & Britton, R. A. Humanized microbiota mice as a model of recurrent Clostridium difficile disease. Microbiome 3, 35 (2015).
29. Sonnenberg, G. F. & Artis, D. Innate lymphoid cell interactions with microbiota: implications for intestinal health and disease. Immunity 37, 601-610 (2012).
30. Watanabe, Y., Nagai, F. & Morotomi, M. Characterization of Phascolarctobacterium succinatutens sp. nov., an asaccharolytic, succinate-utilizing bacterium isolated from human feces. Appl Environ Microbiol 78, 511-518 (2012).
31. Topping, D. L. & Clifton, P. M. Short-chain fatty acids and human colonic function: roles of resistant starch and nonstarch polysaccharides. Physiological reviews 81, 1031-1064 (2001).
32. Ferreyra, J. A. et al. Gut microbiota-produced succinate promotes C. difficile infection after antibiotic treatment or motility disturbance. Cell Host Microbe 16, 770-777 (2014).
33. Rodemann, J. F., Dubberke, E. R., Reske, K. A., Seo da, H. & Stone, C. D. Incidence of Clostridium difficile infection in inflammatory bowel disease. Clin Gastroenterol Hepatol 5, 339-344 (2007).
34. Reddy, S. S. & Brandt, L. J. Clostridium difficile infection and inflammatory bowel disease. J Clin Gastroenterol 47, 666-671 (2013).
35. Berg, A. M., Kelly, C. P. & Farraye, F. A. Clostridium difficile infection in the inflammatory bowel disease patient. Inflamm Bowel Dis 19, 194-204 (2013).
36. Tamboli, C. P., Neut, C., Desreumaux, P. & Colombel, J. F. Dysbiosis in inflammatory bowel disease. Gut 53, 1-4 (2004).
37. Vancamelbeke, M. et al. Genetic and Transcriptomic Bases of Intestinal Epithelial Barrier Dysfunction in Inflammatory Bowel Disease. Inflamm Bowel Dis 23, 1718-1729 (2017).
38. Arijs, I. et al. Mucosal gene expression of antimicrobial peptides in inflammatory bowel disease before and after first infliximab treatment. PLoS One 4, e7984 (2009).
39. Arijs, I. et al. Effect of vedolizumab (anti-alpha4beta7-integrin) therapy on histological healing and mucosal gene expression in patients with UC. Gut 67, 43-52 (2018).
40. Kamada, N., Seo, S. U., Chen, G. Y. & Nunez, G. Role of the gut microbiota in immunity and inflammatory disease. Nat Rev Immunol 13, 321-335 (2013).
41. Johnson, J. L., Jones, M. B., Ryan, S. O. & Cobb, B. A. The regulatory power of glycans and their binding partners in immunity. Trends Immunol 34, 290-298 (2013).
42. Stanley, P. What Have We Learned from Glycosyltransferase Knockouts in Mice? Journal of molecular biology 428, 3166-3182 (2016).
43. Wu, F. et al. Phascolarctobacterium faecium abundant colonization in human gastrointestinal tract. Experimental and therapeutic medicine 14, 3122-3126 (2017).
44. Zhang, L. et al. Insight into alteration of gut microbiota in Clostridium difficile infection and asymptomatic C. difficile colonization. Anaerobe 34, 1-7 (2015).
45. Hourigan, S. K. et al. Microbiome changes associated with sustained eradication of Clostridium difficile after single faecal microbiota transplantation in children with and without inflammatory bowel disease. Aliment Pharmacol Thor 42, 741-752 (2015).
46. Levy, M. et al. Microbiota-Modulated Metabolites Shape the Intestinal Microenvironment by Regulating NLRP6 Inflammasome Signaling. Cell 163, 1428-1443 (2015).
47. Battaglioli, E. J. et al. Clostridioides difficile uses amino acids associated with gut microbial dysbiosis in a subset of patients with diarrhea. Sci Transl Med 10 (2018).
48. Issa, M. et al. Impact of Clostridium difficile on inflammatory bowel disease. Clin Gastroenterol Hepatol 5, 345-351 (2007).
49. Ananthakrishnan, A. N., McGinley, E. L. & Binion, D. G. Excess hospitalisation burden associated with Clostridium difficile in patients with inflammatory bowel disease. Gut 57, 205-210 (2008).
50. Singh, H. et al. Higher Incidence of Clostridium difficile Infection Among Individuals With Inflammatory Bowel Disease. Gastroenterology 153, 430-438.e432 (2017).
51. Morgan, X. C. et al. Dysfunction of the intestinal microbiome in inflammatory bowel disease and treatment. Genome Biol 13, R79 (2012).
52. Wolk, K. et al. IL-22 induces lipopolysaccharide-binding protein in hepatocytes: a potential systemic role of IL-22 in Crohn's disease. J Immunol 178, 5973-5981 (2007).
53. Schmechel, S. et al. Linking genetic susceptibility to Crohn's disease with Th17 cell function: IL-22 serum levels are increased in Crohn's disease and correlate with disease activity and IL23R genotype status. Inflamm Bowel Dis 14, 204-212 (2008).
54. Mann, E. R. et al. Human gut dendritic cells drive aberrant gut-specific t-cell responses in ulcerative colitis, characterized by increased IL-4 production and loss of IL-22 and IFNgamma. Inflamm Bowel Dis 20, 2299-2307 (2014).
55. Leung, J. M. et al. IL-22-producing CD4+ cells are depleted in actively inflamed colitis tissue. Mucosal Immunol 7, 124-133 (2014).
56. Lamas, B. et al. CARD9 impacts colitis by altering gut microbiota metabolism of tryptophan into aryl hydrocarbon receptor ligands. Nat Med 22, 598-605 (2016).
57. Martin, J. C. et al. IL-22BP is produced by eosinophils in human gut and blocks IL-22 protective actions during colitis. Mucosal Immunol 9, 539-549 (2016).
58. Pelczar, P. et al. A pathogenic role for T cell-derived IL-22BP in inflammatory bowel disease. Science 354, 358-362 (2016).
59. Xu, A. T. et al. High suppressor of cytokine signaling-3 expression impairs STAT3-dependent protective effects of interleukin-22 in ulcerative colitis in remission. Inflamm Bowel Dis 21, 241-250 (2015).
60. Li, Y. et al. Increased suppressor of cytokine signaling-3 expression predicts mucosal relapse in ulcerative colitis. Inflamm Bowel Dis 19, 132-140 (2013).
61. Chi, H. G. et al. Association of the interleukin-22 genetic polymorphisms with ulcerative colitis. Diagn Pathol 9, 183 (2014).
62. Jostins, L. et al. Host-microbe interactions have shaped the genetic architecture of inflammatory bowel disease. Nature 491, 119-124 (2012).
63. Dias, A. M. et al. Dysregulation of T cell receptor N-glycosylation: a molecular mechanism involved in ulcerative colitis. Hum Mol Genet 23, 2416-2427 (2014).
64. Lopez, J. & Grinspan, A. Fecal Microbiota Transplantation for Inflammatory Bowel Disease. Gastroenterology & hepatology 12, 374-379 (2016).
65. Pigneur, B. & Sokol, H. Fecal microbiota transplantation in inflammatory bowel disease: the quest for the holy grail. Mucosal Immunol 9, 1360-1365 (2016).
66. Paramsothy, S. et al. Multidonor intensive faecal microbiota transplantation for active ulcerative colitis: a randomised placebo-controlled trial. Lancet 389, 1218-1228 (2017).
67. Atarashi, K. et al. Th17 Cell Induction by Adhesion of Microbes to Intestinal Epithelial Cells. Cell 163, 367-380 (2015).
68. Paik, J. eta]. Potential for using a hermetically-sealed, positive-pressured isocage system for studies involving germ-free mice outside a flexible-film isolator. Gut Microbes 6, 255-265 (2015).
69. Hecht, G. et al. A simple cage-autonomous method for the maintenance of the barrier status of germ-free mice during experimentation. Lab Anim 48, 292-297 (2014).
70. Schloss, P. D. et al. Introducing mothur: open-source, platform-independent, community-supported software for describing and comparing microbial communities. Appl Environ Microbiol 75, 7537-7541 (2009).
71. Kozich, J. J., Westcott, S. L., Baxter, N. T., Highlander, S. K. & Schloss, P. D. Development of a dual-index sequencing strategy and curation pipeline for analyzing amplicon sequence data on the MiSeq Illumina sequencing platform. Appl Environ Microbiol 79, 5112-5120 (2013).
72. Segata, N. et al. Metagenomic biomarker discovery and explanation. Genome Biol 12, R60 (2011).
73. Hirayama, A. et al. Metabolic profiling reveals new serum biomarkers for differentiating diabetic nephropathy. Analytical and bioanalytical chemistry 404, 3101-3109 (2012).
74. Lorenz, M. A., Burant, C. F. & Kennedy, R. T. Reducing time and increasing sensitivity in sample preparation for adherent mammalian cell metabolomics. Analytical chemistry 83, 3406-3414 (2011).
75. Cohen, P. S. & Laux, D. C. Bacterial adhesion to and penetration of intestinal mucus in vitro. Methods in enzymology 253, 309-314 (1995).

All publications and patents mentioned throughout the specification and listed in the above list of references are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A method of treating a subject for a Clostridioides difficile infection, said method comprising:
administering a composition comprising Phascolarctobacterium sp. to a subject in need of treatment for a Clostridioides difficile infection; and
increasing the level of interleukin-22 (IL-22) in the gut of said subject.

2. The method of claim 1 wherein said subject has gut dysbiosis and/or low levels of Phascolarctobacterium spp.

3. The method of claim 1 wherein said subject has a Clostridioides difficile infection (CDI).

4. The method of claim 1 wherein said subject has an increased risk of having a CDI.

5. The method of claim 1 wherein said subject has one or more of diarrhea, ulcerative colitis, colitis, Crohn's disease, irritable bowel disease, and/or inflammatory bowel disease.

6. The method of claim 1 wherein increasing the level of IL-22 in the gut of said subject comprises administering IL-22 to said subject.

7. The method of claim 1 wherein increasing the level of IL-22 in the gut of said subject comprises administering a microorganism to said subject that induces production of IL-22 in the gut.

8. The method of claim 1 wherein increasing the level of IL-22 in the gut of said subject comprises administering a recombinant microorganism that produces IL-22.

9. The method of claim 1 wherein administering a composition comprising Phascolarctobacterium sp. to a subject in need of treatment for a Clostridioides difficile infection and increasing the level of interleukin-22 (IL-22) in the gut of said subject are performed essentially simultaneously.

10. The method of claim 1 wherein administering a composition comprising Phascolarctobacterium sp. to a subject in need of treatment for a Clostridioides difficile infection and increasing the level of interleukin-22 (IL-22) in the gut of said subject are performed sequentially.

11. The method of claim 1 wherein said Phascolarctobacterium sp. is P. faecium and/or P. succinatutens.

12. The method of claim 1 further comprising testing said subject for the level of Phascolarctobacterium sp. in the gut.

13. The method of claim 1 further comprising testing said subject for the level of Clostridioides difficile in the gut.

14. The method of claim 1 further comprising testing said subject for the level of IL-22 in the gut.

15. The method of claim 1 further comprising testing said subject for the level of glycans in the gut.

16. The method of claim 1 further comprising testing said subject for the level of succinate in the gut.

17. The method of claim 1 further comprising testing said subject for the level ofMGAT4A expression or MGAT4A protein in the gut and/or testing said subject for the level ofMGAT4B expression or MGAT4B protein in the gut.

18. The method of claim 1 further comprising obtaining or providing a sample from said subject.

19. A composition comprising Phascolarctobacterium sp. and one or more of:
IL-22;
a recombinant organism expressing IL-22; and/or
an organism that increases IL-22 in the gut.

20-51. (canceled)
